(19) United States
(12) Patent Application Publication
(10) Pub. No.: US 2025/0325663 A1
(43) Pub. Date: Oct. 23, 2025

(54) MEDICAMENT FOR TREATMENT AND/OR PREVENTION OF CANCER

(71) Applicant: TORAY INDUSTRIES, INC., Tokyo (JP)

(72) Inventors: Masahiko KUME, Kamakura-shi (JP); Fumiyoshi OKANO, Kamakura-shi (JP); Takanori SAITO, Kamakura-shi (JP)

(73) Assignee: TORAY INDUSTRIES, INC., Tokyo (JP)

(21) Appl. No.: 18/292,656

(22) PCT Filed: Jul. 27, 2022
(86) PCT No.: PCT/JP2022/028877

§ 371 Date: Jan. 26, 2024

(30) Foreign Application Priority Data
Jul. 27, 2021 (JP) 2021-122071

(51) Int. Cl. A61K 39/395; A61K 45/06; A61P 35/00

(52) U.S. Cl. CPC A61K 39/3955; A61K 45/06; A61P 35/00

(57) ABSTRACT

The present invention relates to a medicament for treatment and/or prevention of cancer, comprising an antibody or a fragment thereof having an immunological reactivity with CAPRIN-1 protein, and an inhibitor of synthesis of N-glycoside-linked sugar chain of a glycoprotein together or separately in combination.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 23, 2024, is named “1254-0688PUS1.xml” and is 388,472 bytes in size. The sequence listing contained in this .XML file is part of the specification and is hereby incorporated by reference herein in its entirety.

### TECHNICAL FIELD

The present invention relates to a medicament for treatment and/or prevention of a cancer, comprising an antibody against CAPRIN-1 protein, or a fragment thereof, and an inhibitor of synthesis of N-glycoside-linked sugar chain of a glycoprotein.

### BACKGROUND ART

Various antibody medicines targeting specific antigen proteins on cancer cells are applied as therapeutic agents for cancers with fewer side effects to cancer treatment because of their cancer specificity. For example, cytoplasmic-activation and proliferation-associated protein 1 (CAPRIN-1) is expressed on cell membrane surfaces of many solid cancers. Antibodies against this CAPRIN-1 protein are known to be promising in pharmaceutical uses for treatment and/or prevention of cancers (Patent Literature 1).

In recent years, treatment methods using combinations of pluralities of therapeutic agents for cancer have been clinically used as standard treatment methods in order to enhance the effectiveness of the therapeutic agents for cancers. In general, for example, breast cancer is treated by a treatment method using a combination of doxorubicin and cyclophosphamide or using a combination of a plurality of anticancer agents such as paclitaxel, trastuzumab, and pertuzumab. Therapeutic agents for cancers comprising anti-CAPRIN-1 antibodies as active ingredients have also been confirmed to have therapeutic effects on the cancers by combinations with chemotherapeutics (Patent Literature 2). However, treatment of a cancer by a combination of chemotherapeutics is not effective for every cancer to which the treatment is applied, and few combinations of chemotherapeutics synergistically drastically enhance therapeutic effects, though some combinations additively enhance therapeutic effects.

Abnormalities in sugar chain modification of proteins are widely recognized as important features of cancers, and increased abnormal sugar chain modification is known to be closely related to the development, progression, metastasis, drug resistance, and immune evasion of cancers (Non Patent Literature 1). In this respect, the sugar chain modification in cancers is considered as an important target for cancer treatment, and inhibitors of the sugar chain modification are regarded as candidates of therapeutic agents for cancers. Preceding research suggests that tunicamycin, a natural product-derived compound inhibiting the synthesis of N-glycoside-linked sugar chain modification of proteins, reduces the proliferation of cancer cells and promotes apoptosis (Non Patent Literature 2). Another research suggests that the N-glycoside-linked sugar chain modification is also important for functions of P-GP (P-glycoprotein) and BCRP (breast cancer resistance protein), which are drug transporters contributing to the drug resistance of cancer cells (Non Patent Literature 3). In recent years, immune checkpoint inhibitors targeting immunomodulatory molecules such as PD-1 (programmed death-1) or PD-L1 (programmed death-ligand 1) are just beginning to be applied to cancer treatment. The N-glycoside-linked sugar chain modification is also important for immunomodulatory functions of such PD-1 or PD-L1, and the inhibition of N-glycoside-linked sugar chain modification of PD-1 or PD-L1 is suggested to suppress the immune evasion of cancer cells (Non Patent Literatures 4 and 5). However, in spite of these basic findings, practical use of N-glycoside-linked sugar chain modification inhibitors as therapeutic agents for cancers has not yet been sufficiently shown, and it is not known what drug is combined with such an inhibitor to produce marked therapeutic effects.

### CITATION LIST

### Patent Literature

Patent Literature 1: WO2010/016526
Patent Literature 2: WO2011/096535

### Non Patent Literature

Non Patent Literature 1: Cancer cell, 2019, 36 (1), 6-16
Non Patent Literature 2: J. Clin. Cancer Res, 2018, 37 (1), 272
Non Patent Literature 3: Biomed. Pharmacother, 2015, 74, 49-56
Non Patent Literature 4: Cancer Res, 2020, 80 (11), 2298-2310
Non Patent Literature 5: Nat. Commun, 2016, 7, 12632

### SUMMARY OF INVENTION

### Object to be Achieved

An object of the present invention is to provide a medicament for treatment and/or prevention of a cancer specifically expressing CAPRIN-1 protein on a cell surface.

### Solution to Achieve Object

As a result of intensive studies, the present inventors have found that a combination (combined use) of an antibody against CAPRIN-1 protein, or a fragment thereof, having an immunological reactivity with cancer cells, and an inhibitor of synthesis of N-glycoside-linked sugar chain of a glycoprotein exerts a very strong antitumor effect. On the basis of these findings, the present invention has been completed.

Specifically, the present invention relates to the following embodiments (1) to (14):
(1) A medicament for treatment and/or prevention of cancer, comprising an antibody or a fragment thereof having an immunological reactivity with CAPRIN-1 protein, and an inhibitor of synthesis of N-glycoside-linked sugar chain of a glycoprotein together or separately in combination.
(2) The medicament according to (1), wherein the inhibitor of synthesis of N-glycoside-linked sugar chain of a glycoprotein is tunicamycin or a pharmaceutically acceptable derivative thereof.
(3) The medicament according to (1) or (2), wherein the antibody or the fragment thereof has an immunological reactivity with CAPRIN-1 protein having an amino acid sequence shown by any one of the even numbered SEQ ID NOs: 2 to 30, or an amino acid sequence having 80% or more sequence identity with the amino acid sequence.
(4) The medicament according to any of (1) to (3), wherein the antibody or the fragment thereof has an immunological reactivity with an extracellular region of CAPRIN-1 protein present on a cancer cell surface.
(5) The medicament according to any of (1) to (4), wherein the antibody or the fragment thereof has an immunological reactivity with a partial polypeptide of CAPRIN-1 protein, the partial polypeptide having an amino acid sequence shown by any one of SEQ ID NOs: 31 to 35, 296 to 299, 308 and 309, or an amino acid sequence having 80% or more sequence identity with the amino acid sequence.
(6) The medicament according to any of (1) to (5), wherein the antibody is a monoclonal antibody or a polyclonal antibody.
(7) The medicament according to any of (1) to (6), wherein the antibody or the fragment thereof is any one of the following (A) to (M):
(A) an antibody or a fragment thereof comprising a heavy-chain variable region comprising complementarity determining regions of SEQ ID NOs: 36, 37 and 38 (CDR1, CDR2 and CDR3, respectively) and a light-chain variable region comprising complementarity determining regions of SEQ ID NOs: 40, 41 and 42 (CDR1, CDR2 and CDR3, respectively), and having an immunological reactivity with CAPRIN-1 protein;
(B) an antibody or a fragment thereof comprising a heavy-chain variable region comprising complementarity determining regions of SEQ ID NOs: 44, 45 and 46 (CDR1, CDR2 and CDR3, respectively) and a light-chain variable region comprising complementarity determining regions of SEQ ID NOs: 48, 49 and 50 (CDR1, CDR2 and CDR3, respectively), and having an immunological reactivity with CAPRIN-1 protein;
(C) an antibody or a fragment thereof comprising a heavy-chain variable region comprising complementarity determining regions of SEQ ID NOs: 52, 53 and 54 (CDR1, CDR2 and CDR3, respectively) and a light-chain variable region comprising complementarity determining regions of SEQ ID NOs: 56, 57 and 58 (CDR1, CDR2 and CDR3, respectively), and having an immunological reactivity with CAPRIN-1 protein;
(D) an antibody or a fragment thereof comprising a heavy-chain variable region comprising complementarity determining regions of SEQ ID NOs: 60, 61 and 62 (CDR1, CDR2 and CDR3, respectively) and a light-chain variable region comprising complementarity determining regions of SEQ ID NOs: 64, 65 and 66 (CDR1, CDR2 and CDR3, respectively), and having an immunological reactivity with CAPRIN-1 protein;
(E) an antibody or a fragment thereof comprising a heavy-chain variable region comprising complementarity determining regions of SEQ ID NOs: 170, 171 and 172 (CDR1, CDR2 and CDR3, respectively) and a light-chain variable region comprising complementarity determining regions of SEQ ID NOs: 173, 174 and 175 (CDR1, CDR2 and CDR3, respectively), and having an immunological reactivity with CAPRIN-1 protein;
(F) an antibody or a fragment thereof comprising a heavy-chain variable region comprising complementarity determining regions of SEQ ID NOs: 176, 177 and 178 (CDR1, CDR2 and CDR3, respectively) and a light-chain variable region comprising complementarity determining regions of SEQ ID NOs: 179, 180 and 181 (CDR1, CDR2 and CDR3, respectively), and having an immunological reactivity with CAPRIN-1 protein;
(G) an antibody or a fragment thereof comprising a heavy-chain variable region comprising complementarity determining regions of SEQ ID NOs: 182, 183 and 184 (CDR1, CDR2 and CDR3, respectively) and a light-chain variable region comprising complementarity determining regions of SEQ ID NOs: 185, 186 and 187 (CDR1, CDR2 and CDR3, respectively), and having an immunological reactivity with CAPRIN-1 protein;
(H) an antibody or a fragment thereof comprising a heavy-chain variable region comprising complementarity determining regions of SEQ ID NOs: 188, 189 and 190 (CDR1, CDR2 and CDR3, respectively) and a light-chain variable region comprising complementarity determining regions of SEQ ID NOs: 191, 192 and 193 (CDR1, CDR2 and CDR3, respectively), and having an immunological reactivity with CAPRIN-1 protein;
(I) an antibody or a fragment thereof comprising a heavy-chain variable region comprising complementarity determining regions of SEQ ID NOs: 146, 147 and 148 (CDR1, CDR2 and CDR3, respectively) and a light-chain variable region comprising complementarity determining regions of SEQ ID NOs: 149, 150 and 151 (CDR1, CDR2 and CDR3, respectively), and having an immunological reactivity with CAPRIN-1 protein;
(J) an antibody or a fragment thereof comprising a heavy-chain variable region comprising complementarity determining regions of SEQ ID NOs: 272, 273 and 274 (CDR1, CDR2 and CDR3, respectively) and a light-chain variable region comprising complementarity determining regions of SEQ ID NOs: 275, 276 and 277 (CDR1, CDR2 and CDR3, respectively), and having an immunological reactivity with CAPRIN-1 protein;
(K) an antibody or a fragment thereof comprising a heavy-chain variable region comprising complementarity determining regions of SEQ ID NOs: 290, 291 and 292 (CDR1, CDR2 and CDR3, respectively) and a light-chain variable region comprising complementarity determining regions of SEQ ID NOs: 293, 294 and 295 (CDR1, CDR2 and CDR3, respectively), and having an immunological reactivity with CAPRIN-1 protein;
(L) an antibody or a fragment thereof comprising a heavy-chain variable region comprising complementarity determining regions of SEQ ID NOs: 301, 302 and 303 (CDR1, CDR2 and CDR3, respectively) and a light-chain variable region comprising complementarity determining regions of SEQ ID NOs: 305, 306 and 307 (CDR1, CDR2 and CDR3, respectively), and having an immunological reactivity with CAPRIN-1 protein; and
(M) an antibody or a fragment thereof comprising a heavy-chain variable region comprising complementarity determining regions of SEQ ID NOs: 134, 135 and 136 (CDR1, CDR2 and CDR3, respectively) and a light-chain variable region comprising complementarity determining regions of SEQ ID NOs: 137, 138 and 139 (CDR1, CDR2 and CDR3, respectively), and having an immunological reactivity with CAPRIN-1 protein.
(8) The medicament according to any of (1) to (7), wherein the antibody or the fragment thereof is any one of the following (a) to (al):
(a) an antibody or a fragment thereof comprising a heavy-chain variable region comprising the amino acid sequence of SEQ ID NO: 39 and a light-chain variable region comprising the amino acid sequence of SEQ ID NO: 43;
(b) an antibody or a fragment thereof comprising a heavy-chain variable region comprising the amino acid sequence of SEQ ID NO: 47 and a light-chain variable region comprising the amino acid sequence of SEQ ID NO: 51;
(c) an antibody or a fragment thereof comprising a heavy-chain variable region comprising the amino acid sequence of SEQ ID NO: 55 and a light-chain variable region comprising the amino acid sequence of SEQ ID NO: 59;
(d) an antibody or a fragment thereof comprising a heavy-chain variable region comprising the amino acid sequence of SEQ ID NO: 63 and a light-chain variable region comprising the amino acid sequence of SEQ ID NO: 67;
(e) an antibody or a fragment thereof comprising a heavy-chain variable region comprising the amino acid sequence of SEQ ID NO: 68 and a light-chain variable region comprising the amino acid sequence of SEQ ID NO: 69;
(f) an antibody or a fragment thereof comprising a heavy-chain variable region comprising the amino acid sequence of SEQ ID NO: 70 and a light-chain variable region comprising the amino acid sequence of SEQ ID NO: 71;
(g) an antibody or a fragment thereof comprising a heavy-chain variable region comprising the amino acid sequence of SEQ ID NO: 72 and a light-chain variable region comprising the amino acid sequence of SEQ ID NO: 73;
(h) an antibody or a fragment thereof comprising a heavy-chain variable region comprising the amino acid sequence of SEQ ID NO: 74 and a light-chain variable region comprising the amino acid sequence of SEQ ID NO: 75;
(i) an antibody or a fragment thereof comprising a heavy-chain variable region comprising the amino acid sequence of SEQ ID NO: 76 and a light-chain variable region comprising the amino acid sequence of SEQ ID NO: 77;
(j) an antibody or a fragment thereof comprising a heavy-chain variable region comprising the amino acid sequence of SEQ ID NO: 78 and a light-chain variable region comprising the amino acid sequence of SEQ ID NO: 79;
(k) an antibody or a fragment thereof comprising a heavy-chain variable region comprising the amino acid sequence of SEQ ID NO: 80 and a light-chain variable region comprising the amino acid sequence of SEQ ID NO: 81;
(l) an antibody or a fragment thereof comprising a heavy-chain variable region comprising the amino acid sequence of SEQ ID NO: 82 and a light-chain variable region comprising the amino acid sequence of SEQ ID NO: 83;
(m) an antibody or a fragment thereof comprising a heavy-chain variable region comprising the amino acid sequence of SEQ ID NO: 84 and a light-chain variable region comprising the amino acid sequence of SEQ ID NO: 85;
(n) an antibody or a fragment thereof comprising a heavy-chain variable region comprising the amino acid sequence of SEQ ID NO: 86 and a light-chain variable region comprising the amino acid sequence of SEQ ID NO: 87;
(o) an antibody or a fragment thereof comprising a heavy-chain variable region comprising the amino acid sequence of SEQ ID NO: 88 and a light-chain variable region comprising the amino acid sequence of SEQ ID NO: 89;
(p) an antibody or a fragment thereof comprising a heavy-chain variable region comprising the amino acid sequence of SEQ ID NO: 90 and a light-chain variable region comprising the amino acid sequence of SEQ ID NO: 91;
(q) an antibody or a fragment thereof comprising a heavy-chain variable region comprising the amino acid sequence of SEQ ID NO: 92 and a light-chain variable region comprising the amino acid sequence of SEQ ID NO: 93;
(r) an antibody or a fragment thereof comprising a heavy-chain variable region comprising the amino acid sequence of SEQ ID NO: 94 and a light-chain variable region comprising the amino acid sequence of SEQ ID NO: 95;
(s) an antibody or a fragment thereof comprising a heavy-chain variable region comprising the amino acid sequence of SEQ ID NO: 96 and a light-chain variable region comprising the amino acid sequence of SEQ ID NO: 97;
(t) an antibody or a fragment thereof comprising a heavy-chain variable region comprising the amino acid sequence of SEQ ID NO: 98 and a light-chain variable region comprising the amino acid sequence of SEQ ID NO: 99;
(u) an antibody or a fragment thereof comprising a heavy-chain variable region comprising the amino acid sequence of SEQ ID NO: 100 and a light-chain variable region comprising the amino acid sequence of SEQ ID NO: 101;
(v) an antibody or a fragment thereof comprising a heavy-chain variable region comprising the amino acid sequence of SEQ ID NO: 102 and a light-chain variable region comprising the amino acid sequence of SEQ ID NO: 103;
(w) an antibody or a fragment thereof comprising a heavy-chain variable region comprising the amino acid sequence of SEQ ID NO: 104 and a light-chain variable region comprising the amino acid sequence of SEQ ID NO: 105;
(x) an antibody or a fragment thereof comprising a heavy-chain variable region comprising the amino acid sequence of SEQ ID NO: 106 and a light-chain variable region comprising the amino acid sequence of SEQ ID NO: 107;
(y) an antibody or a fragment thereof comprising a heavy-chain variable region comprising the amino acid sequence of SEQ ID NO: 108 and a light-chain variable region comprising the amino acid sequence of SEQ ID NO: 109;
(z) an antibody or a fragment thereof comprising a heavy-chain variable region comprising the amino acid sequence of SEQ ID NO: 110 and a light-chain variable region comprising the amino acid sequence of SEQ ID NO: 111;
(aa) an antibody or a fragment thereof comprising a heavy-chain variable region comprising the amino acid sequence of SEQ ID NO: 112 and a light-chain variable region comprising the amino acid sequence of SEQ ID NO: 113;
(ab) an antibody or a fragment thereof comprising a heavy-chain variable region comprising the amino acid sequence of SEQ ID NO: 114 and a light-chain variable region comprising the amino acid sequence of SEQ ID NO: 115;
(ac) an antibody or a fragment thereof comprising a heavy-chain variable region comprising the amino acid sequence of SEQ ID NO: 116 and a light-chain variable region comprising the amino acid sequence of SEQ ID NO: 117;
(ad) an antibody or a fragment thereof comprising a heavy-chain variable region comprising the amino acid sequence of SEQ ID NO: 118 and a light-chain variable region comprising the amino acid sequence of SEQ ID NO: 119;
(ae) an antibody or a fragment thereof comprising a heavy-chain variable region comprising the amino acid sequence of SEQ ID NO: 120 and a light-chain variable region comprising the amino acid sequence of SEQ ID NO: 121;
(af) an antibody or a fragment thereof comprising a heavy-chain variable region comprising the amino acid sequence of SEQ ID NO: 122 and a light-chain variable region comprising the amino acid sequence of SEQ ID NO: 123;
(ag) an antibody or a fragment thereof comprising a heavy-chain variable region comprising the amino acid sequence of SEQ ID NO: 124 and a light-chain variable region comprising the amino acid sequence of SEQ ID NO: 125;
(ah) an antibody or a fragment thereof comprising a heavy-chain variable region comprising the amino acid sequence of SEQ ID NO: 126 and a light-chain variable region comprising the amino acid sequence of SEQ ID NO: 127;
(ai) an antibody or a fragment thereof comprising a heavy-chain variable region comprising the amino acid sequence of SEQ ID NO: 128 and a light-chain variable region comprising the amino acid sequence of SEQ ID NO: 129;
(aj) an antibody or a fragment thereof comprising a heavy-chain variable region comprising the amino acid sequence of SEQ ID NO: 130 and a light-chain variable region comprising the amino acid sequence of SEQ ID NO: 131;
(ak) an antibody or a fragment thereof comprising a heavy-chain variable region comprising the amino acid sequence of SEQ ID NO: 132 and a light-chain variable region comprising the amino acid sequence of SEQ ID NO: 133; and (al) an antibody or a fragment thereof comprising a heavy-chain variable region comprising the amino acid sequence of SEQ ID NO: 300 and a light-chain variable region comprising the amino acid sequence of SEQ ID NO: 304.
(9) The medicament according to any of (1) to (8), wherein the antibody is a human antibody, a humanized antibody, a chimeric antibody or a single chain antibody.
(10) The medicament according to any of (1) to (9), wherein the cancer is a cancer expressing CAPRIN-1 protein on a cell membrane surface.
(11) The medicament according to any of (1) to (10), wherein the cancer is colon cancer, melanoma, lung cancer, prostate cancer, ovarian cancer, kidney cancer, breast cancer, gastric cancer, pancreatic cancer, bile duct cancer, renal cell cancer, Hodgkin's lymphoma, head and neck cancer, mesothelioma, colorectal cancer, esophageal cancer, gastroesophageal cancer, hepatocellular cancer, glioblastoma, urothelial cancer, urinary bladder cancer, uterus cancer, primary central nervous system lymphoma, primary testicular lymphoma, biliary tract cancer, brain tumor, leukemia, lymphoma, liver cancer, sarcoma, fibrosarcoma, mastocytoma, adrenal cortex cancer, Ewing's tumor, multiple myeloma, testicle cancer, thyroid cancer, basal cell cancer, Paget's disease or skin cancer.
(12) An agent increasing drug efficacy of a pharmaceutical composition for treatment and/or prevention of cancer comprising an antibody or a fragment thereof having an immunological reactivity with CAPRIN-1 protein as an active ingredient, wherein the agent comprises an inhibitor of synthesis of N-glycoside-linked sugar chain of a glycoprotein as an active ingredient.
(13) An agent increasing drug efficacy of a pharmaceutical composition for treatment and/or prevention of cancer comprising an inhibitor of synthesis of N-glycoside-linked sugar chain of a glycoprotein as an active ingredient, wherein the agent comprises an antibody or a fragment thereof having an immunological reactivity with CAPRIN-1 protein as an active ingredient.
(14) A method for treating and/or preventing cancer, comprising administering an antibody or a fragment thereof having an immunological reactivity with CAPRIN-1 protein, and an inhibitor of synthesis of N-glycoside-linked sugar chain of a glycoprotein together or separately to a subject.

The present specification encompasses the contents disclosed in Japanese Patent Application No. 2021-122071 on which the priority of the present application is based.

### Advantageous Effects of Invention

The combination of an antibody against CAPRI-1 protein, or a fragment thereof and an inhibitor of synthesis of N-glycoside-linked sugar chain of a glycoprotein according to the present invention exerts a stronger antitumor effect than that of the antibody against CAPRIN-1 protein alone and an existing chemotherapeutic alone. Thus, the combination of the antibody against CAPRIN-1 protein and the inhibitor of synthesis of N-glycoside-linked sugar chain of a glycoprotein is effective for treatment or prevention of cancer.

### BRIEF DESCRIPTION OF DRAWINGS

FIG. 1 is a diagram showing human monocytic cell (THP-1)-mediated phagocytic activity of a combination of an anti-CAPRIN-1 antibody and tunicamycin against a human cancer cell line. Reference number 1 depicts the phagocytic activity against a human colon cancer cell line (HCT116), reference number 2 depicts the phagocytic activity against a human melanoma cell line (A375), reference number 3 depicts the phagocytic activity against a human lung cancer cell line (A549), and reference number 4 depicts the phagocytic activity against a human prostate cancer cell line (DU145). Shaded bar graph: a test group without a drug used in combination (DMSO was added instead of the drug). Filled bar graph; a tunicamycin combination test group.

FIG. 2 is a diagram showing human monocytic cell (THP-1)-mediated phagocytic activity of a combination of an anti-CAPRIN-1 antibody and each drug against a human colon cancer cell line HCT116. Reference number 5 depicts the phagocytic activity in a test group without a drug used in combination (DMSO was added instead of the drug to the cells), reference number 6 depicts the phagocytic activity in a tunicamycin combination test group (0.5 μM), reference number 7 depicts the phagocytic activity in a fluorouracil (5-FU) combination test group (0.4 μg/mL), and reference number 8 depicts the phagocytic activity in an irinotecan combination test group (15 μM).

### DESCRIPTION OF EMBODIMENTS

The antitumor effect of the combination of an antibody against CAPRIN-1 protein or a fragment thereof (hereinafter, referred to as an “anti-CAPRIN-1 antibody”) and an inhibitor of synthesis of N-glycoside-linked sugar chain of a glycoprotein, used in the present invention is preferably evaluated by examining in vitro the phagocytic activity against cancer cells by immunocytes in coculturing the cancer cells and the immunocytes as mentioned later. The immunocyte used in evaluating the antitumor effect in vitro may be any cell as long as the immunocyte is a blood cell having phagocytic activity, and preferably, is a human monocytic cell (THP-1 or U937). When an antibody binds to a cancer cell, this is recognized by an immunocyte so that the cancer cell is killed via phagocytic activity by the immunocyte. Therefore, an in vivo antitumor effect can be predicted by evaluating the in vitro antitumor effect.

The term “combination” (or “combined use”) described herein refers to simultaneous administration or addition, or administration or addition in a predetermined interval of the anti-CAPRIN-1 antibody and the inhibitor of synthesis of N-glycoside-linked sugar chain of a glycoprotein as independent active ingredients to the same organism or cell. The interval may be simultaneous administration or may be 30 minutes later, 1 hour later, 3 hours later, 6 hours later, 12 hours later, 1 day later, 2 days later, 3 days later, 5 days later, 7 days later, 2 weeks later, 3 weeks later, or 4 weeks later. During one of the anti-CAPRIN-1 antibody or the inhibitor of synthesis of N-glycoside-linked sugar chain of a glycoprotein exhibits its antitumor effect, the other can be administered or added.

The term “comprising together or separately in combination” described herein refers to comprising a plurality of drugs in a form that allows the drugs to be administered simultaneously or separately to a patient. The form may be, for example, the form of a so-called mixed formulation in which a plurality of drugs are mixed, or may be the form of a so-called kit formulation (pharmaceutical kit) comprising a plurality of drugs as individual formulations. The form also encompasses the form of a kit formulation comprising a plurality of drugs in any combination in two or more of formulations.

Such a kit formulation according to the present invention may be, for example, a kit formulation comprising: a formulation (or a pharmaceutical composition) comprising the anti-CAPRIN-1 antibody and a formulation (or a pharmaceutical composition) comprising the inhibitor of synthesis of N-glycoside-linked sugar chain of a glycoprotein.

The anti-CAPRIN-1 antibody according to the present invention may be a monoclonal antibody or a polyclonal antibody and is preferably a monoclonal antibody. The antibody of the present invention may be any type of antibody as long as it can exhibit antitumor effect. The antibody may be a recombinant antibody, a human antibody, a humanized antibody, a chimeric antibody, or a non-human animal antibody.

Subjects being subjected to treatment and/or prevention of cancer according to the present invention are mammals such as primates, pet animals, livestock animals, or sport animals and preferably humans, dogs and cats, and more preferably humans.

Medicaments comprising an anti-CAPRIN-1 antibody and an inhibitor of synthesis of N-glycoside-linked sugar chain of a glycoprotein as active ingredients, and methods for treating and/or preventing cancers, related to the present invention, will be explained below.

### <Anti-CAPRIN-1 Antibody>

Among CAPRIN-1 proteins having an amino acid sequence shown by any one of the even numbered SEQ ID NOs: 2 to 30, which are specific examples of antigens having an immunological reactivity with the anti-CAPRIN-1 antibody used in the present invention, the amino acid sequences shown by SEQ ID NOs: 6, 8, 10, 12 and 14 are amino acid sequences of canine CAPRIN-1 proteins; the amino acid sequences shown by SEQ ID NOs: 2 and 4 are amino acid sequences of human CAPRIN-1 proteins; the amino acid sequence shown by SEQ ID NO: 16 is an amino acid sequence of a bovine CAPRIN-1 protein; the amino acid sequence shown by SEQ ID NO: 18 is an amino acid sequence of a horse CAPRIN-1 protein; the amino acid sequences shown by SEQ ID NOs: 20, 22, 24, 26 and 28 are amino acid sequences of mouse CAPRIN-1 proteins; and the amino acid sequence shown by SEQ ID NO: 30 is an amino acid sequence of a chicken CAPRIN-1 protein.

The anti-CAPRIN-1 antibody used in the present invention may have an immunological reactivity with a CAPRIN-1 protein variant having 80% or more, preferably 90% or more, more preferably 95% or more, and further preferably 99% or more sequence identity to the amino acid sequence shown by any one of the even numbered SEQ ID NOs: 2 to 30. The term “% sequence identity” as used herein means a percentage (%) of the number of identical amino acids (or nucleotides) to the total number of amino acids (or nucleotides) in the case that two sequences are aligned such that maximum similarity can be achieved with or without introduction of gaps.

In the present invention, the anti-CAPRIN-1 antibody refers to an antibody or a fragment (antigen binding fragment) thereof having an immunological reactivity with a full-length CAPRIN-1 protein or a fragment thereof. The term “immunological reactivity” used herein indicates the characteristics of an antibody specifically binding in vivo to CAPRIN-1 protein or a partial polypeptide thereof.

The anti-CAPRIN-1 antibody used in the present invention may be a monoclonal antibody or a polyclonal antibody.

Polyclonal antibodies having an immunological reactivity with a full-length CAPRIN-1 protein or a fragment thereof (anti-CAPRIN-1 polyclonal antibodies) can be obtained, for example, in a manner described below. Serum is obtained after mice, human antibody-producing mice, rats, rabbits, chickens, or the like are immunized using naturally occurring CAPRIN-1 protein or the protein fused with GST or the like, or a partial peptide thereof. The obtained serum is purified via ammonium sulfate precipitation, protein A, protein G, DEAE ion-exchange columns, affinity columns to which a CAPRIN-1 protein or a partial peptide thereof is coupled, or the like.

Nucleotide sequences and amino acid sequences of CAPRIN-1 and homologs thereof used in the immunization can be obtained by, for example, accessing the website of GenBank (NCBI, USA) and using the BLAST or FASTA algorithm (Karlin and Altschul, Proc. Natl. Acad. Sci. USA, 90:5873-5877, 1993; and Altschul et al., Nucleic Acids Res. 25:3389-3402, 1997). Methods for producing CAPRIN-1 protein can be obtained with reference to WO2014/012479 or may employ cells or the like expressing CAPRIN-1 protein.

Monoclonal antibodies having an immunological reactivity with a full-length CAPRIN-1 protein or a fragment thereof (anti-CAPRIN-1 monoclonal antibodies) can be obtained, for example, in a manner described below. Breast cancer cells SK-BR-3 expressing CAPRIN-1, a full-length CAPRIN-1 protein or a fragment thereof, or the like is administered to mice for immunization. Splenocytes separated from the mice are fused with myeloma cells. Clones capable of producing anti-CAPRIN-1 monoclonal antibodies can be selected from the obtained fusion cells (hybridomas) to obtain these antibodies. The antibodies produced from the selected hybridomas can be obtained in the same way as the aforementioned method for purifying polyclonal antibodies.

The antibody used in the present invention includes human antibodies, humanized antibodies, chimeric antibodies, non-human animal antibodies, and single chain antibodies.

For human antibodies, human lymphocytes infected with EB virus are sensitized with a protein, protein-expressing cells, or a lysate thereof. The sensitized lymphocytes are fused with human-derived myeloma cells such as U266 cells. Antibodies having an immunological reactivity with a full-length CAPRIN-1 protein or a fragment thereof can be obtained from the obtained fusion cells.

A humanized antibody is a modified antibody, and it is sometimes referred to as a reshaped human antibody. It is known that a humanized antibody is constructed by transplanting complementarity determining regions of an immunized animal-derived antibody into complementarity determining regions of a human antibody. In addition, a general gene recombinant technique therefor is well known. Specifically, a DNA sequence designed in a manner that allows complementarity determining regions of mouse or rabbit antibody to be ligated to human antibody framework regions is synthesized by the PCR method using several oligonucleotides prepared in such a manner that the oligonucleotides have portions overlapping each other at an end of each thereof. A humanized antibody can be obtained by ligating the above obtained DNA to DNA encoding a human antibody constant region, incorporating the resultant into an expression vector, and introducing the vector into a host for antibody production (see EP-A-239400 and WO96/02576). Framework regions of human antibody ligated to each other via complementarity determining regions are selected on the assumption that complementarity determining regions can form a good antigen binding site. If necessary, amino acids in framework regions of an antibody variable region may be substituted in such a manner that complementarity determining regions in a reshaped human antibody form an appropriate antigen binding site (Sato K. et al., Cancer Research 1993, 53:851-856). In addition, the framework regions may be substituted with framework regions from a different human antibody (see WO99/51743).

In general, antibodies are heteromultimeric glycoproteins each comprising at least two heavy chains and two light chains. Antibodies each comprise two identical light chains and two identical heavy chains. Each heavy chain has a heavy-chain variable region at one end thereof, to which some constant regions are bound in series. Each light chain has a light-chain variable region at one end thereof to which some constant regions are bound in series. Variable regions have specific variable regions, which are called complementarity determining regions (CDRs) and impart binding specificity to an antibody. A relatively conserved portion in a variable region is called a framework region (FR). A complete heavy-chain or light-chain variable region comprises 4 FRs connected to each other via 3 CDRs (CDR1 to CDR3).

Sequences of human-derived heavy-chain and light-chain constant regions and variable regions can be obtained from, for example, NCBI (USA; GenBank, UniGene, etc.). For example, for a human IgG1 heavy-chain constant region, see registration No. J00228; for a human IgG2 heavy-chain constant region, see registration No. J00230; for a human light chain κ constant region, see sequences such as registration Nos. V00557, X64135, and X64133; and for a human light chain λ constant region, see sequences such as registration Nos. X64132 and X64134.

A chimeric antibody is an antibody produced by combining sequences from different animals. An example thereof is an antibody consisting of mouse antibody heavy-chain and light-chain variable regions and constant regions of human antibody heavy-chain and light-chain variable regions. Such a chimeric antibody can be produced by a known method. For example, it can be obtained by ligating DNA encoding an antibody V region to DNA encoding a human antibody C region, incorporating the resultant into an expression vector, and introducing the vector into a host for antibody production.

Non-human animal antibodies are obtained by immunizing non-human animals with sensitizing antigens according to a known method or by intraperitoneally, intracutaneously, or subcutaneously injecting sensitizing antigens into animals such as mice as a general method. For injecting sensitizing antigens, an appropriate amount of various adjuvants including CFA (Freund's complete adjuvant) is mixed therewith and the mixture is administered to animals several times. After immunization of animals and confirmation of an anti-CAPRIN-1 antibody contained in serum, the serum is obtained and the non-human animal antibody can be obtained by purification via ammonium sulfate precipitation, protein A, protein G, DEAE ion-exchange columns, affinity columns to which a CAPRIN-1 protein or a partial peptide thereof is coupled, or the like, as mentioned above. In the case of obtaining monoclonal antibodies from non-human animals, a monoclonal antibody can be obtained by collecting immunocytes from the immunized animals and subjected to cell fusion with myeloma cells. The cell fusion of immunocytes with myeloma cells can be carried out according to a known method (see Kohler, G. and Milstein, C. Methods Enzymol. (1981) 73, 3-46).

The antibody used in the present invention can also be obtained as a gene recombinant antibody produced by cloning an antibody gene from a hybridoma, incorporating the clone into an adequate vector, introducing the vector into a host, and producing the antibody by using a gene recombinant technique (see Carl, A. K. Borrebaeck, James, W. Larrick, THERAPEUTIC MONOCLONAL ANTIBODIES, Published in the United Kingdom by MACMILLAN PUBLISHERS LTD, 1990).

Amino acids in a variable region (e.g., FR) or a constant region in the anti-CAPRIN-1 antibody used in the present invention may be substituted with different amino acids. The amino acid substitution is a substitution of 1 or more, for example, less than 15, less than 10, not more than 8, not more than 6, not more than 5, not more than 4, not more than 3, or not more than 2 amino acids, preferably 1 to 9 amino acids. A substituted antibody should have characteristics of specifically binding to the antigen and binding affinity for the antigen equivalent to or more than those of an unsubstituted antibody and should be an antibody that causes no rejection when applied to humans. The amino acid substitution is preferably a conservative amino acid substitution, which is a substitution between amino acids having similar characteristics in terms of charge, side chains, polarity, aromaticity, and the like. For example, characteristically similar amino acids can be classified into the following types: basic amino acids (arginine, lysine, and histidine); acidic amino acids (aspartic acid and glutamic acid); uncharged polar amino acids (glycine, asparagine, glutamine, serine, threonine, cysteine, and tyrosine); nonpolar amino acids (leucine, isoleucine, alanine, valine, proline, phenylalanine, tryptophan, and methionine); branched-chain amino acids (threonine, valine, isoleucine); and aromatic amino acids (phenylalanine, tyrosine, tryptophan, and histidine).

The anti-CAPRIN-1 antibody used in the present invention is expected to have a stronger antitumor effect when having higher binding affinity for CAPRIN-1 protein on cancer cell surfaces. Association constant (affinity constant) Ka (kon/koff) is preferably at least 10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, or at least 10¹³ M⁻¹.

The anti-CAPRIN-1 antibody used in the present invention may be chemically modified. Examples of such an antibody modifier can include antibodies bound to various molecules such as polyethylene glycol (PEG) and antitumor compounds (for example, antitumor agents listed below). Regarding antibody modifiers of the present invention, substances that bind to an antibody are not limited. Such an antibody modifier can be obtained by chemically modifying an obtained antibody. Methods of such modification have been already established in the field related to the present invention.

The binding strength of the anti-CAPRIN-1 antibody used in the present invention against effector cells can be improved by substituting 1, 2 or several amino acids in the heavy chain-constant region of the antibody or by removing fucose bound to N-acetylglucosamine in a N-glycoside-linked sugar chain bound to the heavy-chain constant region. The anti-CAPRIN-1 antibody described above may have the amino acid substitution alone or may be a composition with an antibody bound to fucose.

Antibodies in which 1, 2 or several amino acids in the heavy-chain constant region have been substituted can be produced with reference to, for example, WO2004/063351, WO2011/120135, U.S. Pat. No. 8,388,955, WO2011/005481, U.S. Pat. No. 6,737,056, and WO2005/063351.

Antibodies in which fucose bound to N-acetylglucosamine in a N-glycoside-linked sugar chain in the heavy-chain constant region has been removed, or producing cells thereof can be produced with reference to U.S. Pat. No. 6,602,684, EP U.S. Pat. No. 1,914,244, and U.S. Pat. No. 7,579,170. Compositions of antibodies in which fucose bound to N-acetylglucosamine in a N-glycoside-linked sugar chain bound to the heavy-chain constant region has been removed, with antibodies bound to fucose, or producing cells thereof can be produced with reference to, for example, U.S. Pat. No. 8,642,292.

The anti-CAPRIN-1 polyclonal antibody and the anti-CAPRIN-1 monoclonal antibody used in the present invention, methods for producing or purifying antibodies and methods for producing CAPRIN-1 protein or partial polypeptide thereof used in immunization can be obtained with reference to WO2010/016526, WO2011/096517, WO2011/096528, WO2011/096519, WO2011/096533, WO2011/096534, WO2011/096535, WO2013/018886, WO2013/018894, WO2013/018892, WO2013/018891, WO2013/018889, WO2013/018883, WO2013/125636, WO2013/125654, WO2013/125630, WO2013/125640, WO2013/147169, WO2013/147176 and WO2015/020212.

Specific examples of the anti-CAPRIN-1 antibody according to the present invention include anti-CAPRIN-1 antibodies described in WO2010/016526, WO2011/096517, WO2011/096528, WO2011/096519, WO2011/096533, WO2011/096534, WO2011/096535, WO2013/018886, WO2013/018894, WO2013/018892, WO2013/018891, WO2013/018889, WO2013/018883, WO2013/125636, WO2013/125654, WO2013/125630, WO2013/125640, WO2013/147169, WO2013/147176 and WO2015/020212 mentioned above. Preferred examples of the anti-CAPRIN-1 antibody include the following.

An antibody or a fragment thereof having an immunological reactivity with a partial polypeptide of CAPRIN-1 protein having the amino acid sequence shown by SEQ ID NO: 2 or SEQ ID NO: 4 or an amino acid sequence having 80% or more (preferably 85% or more, more preferably 90% or more, further preferably 95% or more, and still further preferably 99% or more) sequence identity to the amino acid sequence.

An antibody or a fragment thereof having an immunological reactivity with a partial polypeptide of CAPRIN-1 protein, the partial polypeptide having the amino acid sequence shown by SEQ ID NO: 31 or an amino acid sequence having 80% or more (preferably 85% or more, more preferably 90% or more, and further preferably 95% or more) sequence identity to the amino acid sequence, preferably an antibody or a fragment thereof comprising a heavy-chain variable region comprising complementarity determining regions of SEQ ID NOs: 36, 37 and 38 (CDR1, CDR2 and CDR3, respectively) and a light-chain variable region comprising complementarity determining regions of SEQ ID NOs: 40, 41 and 42 (CDR1, CDR2 and CDR3, respectively), and having an immunological reactivity with CAPRIN-1 protein, an antibody or a fragment thereof comprising a heavy-chain variable region comprising complementarity determining regions of SEQ ID NOs: 140, 141 and 142 (CDR1, CDR2 and CDR3, respectively) and a light-chain variable region comprising complementarity determining regions of SEQ ID NOs: 143, 144 and 145 (CDR1, CDR2 and CDR3, respectively), and having an immunological reactivity with CAPRIN-1 protein, or an antibody or a fragment thereof comprising a heavy-chain variable region comprising complementarity determining regions of SEQ ID NOs: 164, 165 and 166 (CDR1, CDR2 and CDR3, respectively) and a light-chain variable region comprising complementarity determining regions of SEQ ID NOs: 167, 168 and 169 (CDR1, CDR2 and CDR3, respectively), and having an immunological reactivity with CAPRIN-1 protein, and more preferably an antibody or a fragment thereof comprising a heavy-chain variable region comprising the amino acid sequence of SEQ ID NO: 39 and a light-chain variable region comprising the amino acid sequence of SEQ ID NO: 43, an antibody or a fragment thereof comprising a heavy-chain variable region comprising the amino acid sequence of SEQ ID NO: 70 and a light-chain variable region comprising the amino acid sequence of SEQ ID NO: 71, or an antibody or a fragment thereof comprising a heavy-chain variable region comprising the amino acid sequence of SEQ ID NO: 78 and a light-chain variable region comprising the amino acid sequence of SEQ ID NO: 79.

An antibody or a fragment thereof having an immunological reactivity with a partial polypeptide of CAPRIN-1 protein, the partial polypeptide having the amino acid sequence shown by SEQ ID NO: 33 or an amino acid sequence having 80% or more (preferably 85% or more, more preferably 90% or more, and further preferably 95% or more) sequence identity to the amino acid sequence, preferably an antibody or a fragment thereof comprising a heavy-chain variable region comprising complementarity determining regions of SEQ ID NOs: 60, 61 and 62 (CDR1, CDR2 and CDR3, respectively) and a light-chain variable region comprising complementarity determining regions of SEQ ID NOs: 64, 65 and 66 (CDR1, CDR2 and CDR3, respectively), and having an immunological reactivity with CAPRIN-1 protein, and more preferably an antibody or a fragment thereof comprising a heavy-chain variable region comprising the amino acid sequence of SEQ ID NO: 63 and a light-chain variable region comprising the amino acid sequence of SEQ ID NO: 67.

An antibody or a fragment thereof having an immunological reactivity with a partial polypeptide of CAPRIN-1 protein, the partial polypeptide having the amino acid sequence shown by SEQ ID NO: 32 or an amino acid sequence having 80% or more (preferably 85% or more, more preferably 90% or more, and further preferably 95% or more) sequence identity to the amino acid sequence, preferably an antibody or a fragment thereof comprising a heavy-chain variable region comprising complementarity determining regions of SEQ ID NOs: 52, 53 and 54 (CDR1, CDR2 and CDR3, respectively) and a light-chain variable region comprising complementarity determining regions of SEQ ID NOs: 56, 57 and 58 (CDR1, CDR2 and CDR3, respectively), and having an immunological reactivity with CAPRIN-1 protein, and more preferably an antibody or a fragment thereof comprising a heavy-chain variable region comprising the amino acid sequence of SEQ ID NO: 55 and a light-chain variable region comprising the amino acid sequence of SEQ ID NO: 59.

An antibody or a fragment thereof having an immunological reactivity with a partial polypeptide of CAPRIN-1 protein, the partial polypeptide having the amino acid sequence shown by SEQ ID NO: 34 or an amino acid sequence having 80% or more (preferably 85% or more, more preferably 90% or more, and further preferably 95% or more) sequence identity to the amino acid sequence, preferably an antibody or a fragment thereof comprising a heavy-chain variable region comprising complementarity determining regions of SEQ ID NOs: 170, 171 and 172 (CDR1, CDR2 and CDR3, respectively) and a light-chain variable region comprising complementarity determining regions of SEQ ID NOs: 173, 174 and 175 (CDR1, CDR2 and CDR3, respectively), and having an immunological reactivity with CAPRIN-1 protein, or an antibody or a fragment thereof comprising a heavy-chain variable region comprising complementarity determining regions of SEQ ID NOs: 176, 177 and 178 (CDR1, CDR2 and CDR3, respectively) and a light-chain variable region comprising complementarity determining regions of SEQ ID NOs: 179, 180 and 181 (CDR1, CDR2 and CDR3, respectively), and having an immunological reactivity with CAPRIN-1 protein, and more preferably an antibody or a fragment thereof comprising a heavy-chain variable region comprising the amino acid sequence of SEQ ID NO: 80 and a light-chain variable region comprising the amino acid sequence of SEQ ID NO: 81, or an antibody or a fragment thereof comprising a heavy-chain variable region comprising the amino acid sequence of SEQ ID NO: 82 and a light-chain variable region comprising the amino acid sequence of SEQ ID NO: 83.

An antibody or a fragment thereof having an immunological reactivity with a partial polypeptide of CAPRIN-1 protein, the partial polypeptide having the amino acid sequence shown by SEQ ID NO: 35 or an amino acid sequence having 80% or more (preferably 85% or more, more preferably 90% or more, and further preferably 95% or more) sequence identity to the amino acid sequence, preferably an antibody or a fragment thereof comprising a heavy-chain variable region comprising complementarity determining regions of SEQ ID NOs: 182, 183 and 184 (CDR1, CDR2 and CDR3, respectively) and a light-chain variable region comprising complementarity determining regions of SEQ ID NOs: 185, 186 and 187 (CDR1, CDR2 and CDR3, respectively), and having an immunological reactivity with CAPRIN-1 protein, or an antibody or a fragment thereof comprising a heavy-chain variable region comprising complementarity determining regions of SEQ ID NOs: 188, 189 and 190 (CDR1, CDR2 and CDR3, respectively) and a light-chain variable region comprising complementarity determining regions of SEQ ID NOs: 191, 192 and 193 (CDR1, CDR2 and CDR3, respectively), and having an immunological reactivity with CAPRIN-1 protein, and more preferably an antibody or a fragment thereof comprising a heavy-chain variable region comprising the amino acid sequence of SEQ ID NO: 84 and a light-chain variable region comprising the amino acid sequence of SEQ ID NO: 85, or an antibody or a fragment thereof comprising a heavy-chain variable region comprising the amino acid sequence of SEQ ID NO: 86 and a light-chain variable region comprising the amino acid sequence of SEQ ID NO: 87.

An antibody or a fragment thereof comprising a heavy-chain variable region comprising complementarity determining regions of SEQ ID NOs: 44, 45 and 46 (CDR1, CDR2 and CDR3, respectively) and a light-chain variable region comprising complementarity determining regions of SEQ ID NOs: 48, 49 and 50 (CDR1, CDR2 and CDR3, respectively), and having an immunological reactivity with CAPRIN-1 protein, and preferably an antibody or a fragment thereof comprising a heavy-chain variable region comprising the amino acid sequence of SEQ ID NO: 47 and a light-chain variable region comprising the amino acid sequence of SEQ ID NO: 51.

An antibody or a fragment thereof having an immunological reactivity with a partial polypeptide of CAPRIN-1 protein, the partial polypeptide having the amino acid sequence shown by SEQ ID NO: 296 or an amino acid sequence having 80% or more (preferably 85% or more, more preferably 90% or more, and further preferably 95% or more) sequence identity to the amino acid sequence, preferably an antibody or a fragment thereof comprising a heavy-chain variable region comprising complementarity determining regions of SEQ ID NOs: 146, 147 and 148 (CDR1, CDR2 and CDR3, respectively) and a light-chain variable region comprising complementarity determining regions of SEQ ID NOs: 149, 150 and 151 (CDR1, CDR2 and CDR3, respectively), and having an immunological reactivity with CAPRIN-1 protein, and more preferably an antibody or a fragment thereof comprising a heavy-chain variable region comprising the amino acid sequence of SEQ ID NO: 72 and a light-chain variable region comprising the amino acid sequence of SEQ ID NO: 73.

An antibody or a fragment thereof having an immunological reactivity with a partial polypeptide of CAPRIN-1 protein, the partial polypeptide having the amino acid sequence shown by SEQ ID NO: 297 or an amino acid sequence having 80% or more (preferably 85% or more, more preferably 90% or more, and further preferably 95% or more) sequence identity to the amino acid sequence, preferably an antibody or a fragment thereof comprising a heavy-chain variable region comprising complementarity determining regions of SEQ ID NOs: 272, 273 and 274 (CDR1, CDR2 and CDR3, respectively) and a light-chain variable region comprising complementarity determining regions of SEQ ID NOs: 275, 276 and 277 (CDR1, CDR2 and CDR3, respectively), and having an immunological reactivity with CAPRIN-1 protein, and more preferably an antibody or a fragment thereof comprising a heavy-chain variable region comprising the amino acid sequence of SEQ ID NO: 114 and a light-chain variable region comprising the amino acid sequence of SEQ ID NO: 115.

An antibody or a fragment thereof having an immunological reactivity with a partial polypeptide of CAPRIN-1 protein, the partial polypeptide having the amino acid sequence shown by SEQ ID NO: 298 or an amino acid sequence having 80% or more (preferably 85% or more, more preferably 90% or more, and further preferably 95% or more) sequence identity to the amino acid sequence, preferably an antibody or a fragment thereof comprising a heavy-chain variable region comprising complementarity determining regions of SEQ ID NOs: 290, 291 and 292 (CDR1, CDR2 and CDR3, respectively) and a light-chain variable region comprising complementarity determining regions of SEQ ID NOs: 293, 294 and 295 (CDR1, CDR2 and CDR3, respectively), and having an immunological reactivity with CAPRIN-1 protein, and more preferably an antibody or a fragment thereof comprising a heavy-chain variable region comprising the amino acid sequence of SEQ ID NO: 120 and a light-chain variable region comprising the amino acid sequence of SEQ ID NO: 121.

An antibody or a fragment thereof having an immunological reactivity with a partial polypeptide of CAPRIN-1 protein, the partial polypeptide having the amino acid sequence shown by SEQ ID NO: 299 or an amino acid sequence having 80% or more (preferably 85% or more, more preferably 90% or more, and further preferably 95% or more) sequence identity to the amino acid sequence, preferably an antibody or a fragment thereof comprising a heavy-chain variable region comprising complementarity determining regions of SEQ ID NOs: 301, 302 and 303 (CDR1, CDR2 and CDR3, respectively) and a light-chain variable region comprising complementarity determining regions of SEQ ID NOs: 305, 306 and 307 (CDR1, CDR2 and CDR3, respectively), and having an immunological reactivity with CAPRIN-1 protein, and more preferably an antibody or a fragment thereof comprising a heavy-chain variable region comprising the amino acid sequence of SEQ ID NO: 300 and a light-chain variable region comprising the amino acid sequence of SEQ ID NO: 304.

An antibody or a fragment thereof having an immunological reactivity with a partial polypeptide of CAPRIN-1 protein, the partial polypeptide having the amino acid sequence shown by SEQ ID NO: 308 or an amino acid sequence having 80% or more (preferably 85% or more, more preferably 90% or more, and further preferably 95% or more) sequence identity to the amino acid sequence, preferably an antibody or a fragment thereof comprising a heavy-chain variable region comprising complementarity determining regions of SEQ ID NOs: 134, 135 and 136 (CDR1, CDR2 and CDR3, respectively) and a light-chain variable region comprising complementarity determining regions of SEQ ID NOs: 137, 138 and 139 (CDR1, CDR2 and CDR3, respectively), and having an immunological reactivity with CAPRIN-1 protein, and more preferably an antibody or a fragment thereof comprising a heavy-chain variable region comprising the amino acid sequence of SEQ ID NO: 68 and a light-chain variable region comprising the amino acid sequence of SEQ ID NO: 69.

An antibody or a fragment thereof having an immunological reactivity with a partial polypeptide of CAPRIN-1 protein, the partial polypeptide having the amino acid sequence shown by SEQ ID NO: 309 or an amino acid sequence having 80% or more (preferably 85% or more, more preferably 90% or more, and further preferably 95% or more) sequence identity to the amino acid sequence, preferably an antibody or a fragment thereof comprising a heavy-chain variable region comprising complementarity determining regions of SEQ ID NOs: 134, 135 and 136 (CDR1, CDR2 and CDR3, respectively) and a light-chain variable region comprising complementarity determining regions of SEQ ID NOs: 137, 138 and 139 (CDR1, CDR2 and CDR3, respectively), and having an immunological reactivity with CAPRIN-1 protein, and more preferably an antibody or a fragment thereof comprising a heavy-chain variable region comprising the amino acid sequence of SEQ ID NO: 68 and a light-chain variable region comprising the amino acid sequence of SEQ ID NO: 69.

In addition, the following anti-CAPRIN-1 antibodies are also preferably used.

An antibody or a fragment thereof comprising a heavy-chain variable region comprising the amino acid sequence of SEQ ID NO: 68 and a light-chain variable region comprising the amino acid sequence of SEQ ID NO: 69.

An antibody or a fragment thereof comprising a heavy-chain variable region comprising the amino acid sequence of SEQ ID NO: 70 and a light-chain variable region comprising the amino acid sequence of SEQ ID NO: 71.

An antibody or a fragment thereof comprising a heavy-chain variable region comprising the amino acid sequence of SEQ ID NO: 72 and a light-chain variable region comprising the amino acid sequence of SEQ ID NO: 73.

An antibody or a fragment thereof comprising a heavy-chain variable region comprising the amino acid sequence of SEQ ID NO: 74 and a light-chain variable region comprising the amino acid sequence of SEQ ID NO: 75.

An antibody or a fragment thereof comprising a heavy-chain variable region comprising the amino acid sequence of SEQ ID NO: 76 and a light-chain variable region comprising the amino acid sequence of SEQ ID NO: 77.

An antibody or a fragment thereof comprising a heavy-chain variable region comprising the amino acid sequence of SEQ ID NO: 78 and a light-chain variable region comprising the amino acid sequence of SEQ ID NO: 79.

An antibody or a fragment thereof comprising a heavy-chain variable region comprising the amino acid sequence of SEQ ID NO: 80 and a light-chain variable region comprising the amino acid sequence of SEQ ID NO: 81.

An antibody or a fragment thereof comprising a heavy-chain variable region comprising the amino acid sequence of SEQ ID NO: 82 and a light-chain variable region comprising the amino acid sequence of SEQ ID NO: 83.

An antibody or a fragment thereof comprising a heavy-chain variable region comprising the amino acid sequence of SEQ ID NO: 84 and a light-chain variable region comprising the amino acid sequence of SEQ ID NO: 85.

An antibody or a fragment thereof comprising a heavy-chain variable region comprising the amino acid sequence of SEQ ID NO: 86 and a light-chain variable region comprising the amino acid sequence of SEQ ID NO: 87.

An antibody or a fragment thereof comprising a heavy-chain variable region comprising the amino acid sequence of SEQ ID NO: 88 and a light-chain variable region comprising the amino acid sequence of SEQ ID NO: 89.

An antibody or a fragment thereof comprising a heavy-chain variable region comprising the amino acid sequence of SEQ ID NO: 90 and a light-chain variable region comprising the amino acid sequence of SEQ ID NO: 91.

An antibody or a fragment thereof comprising a heavy-chain variable region comprising the amino acid sequence of SEQ ID NO: 92 and a light-chain variable region comprising the amino acid sequence of SEQ ID NO: 93.

An antibody or a fragment thereof comprising a heavy-chain variable region comprising the amino acid sequence of SEQ ID NO: 94 and a light-chain variable region comprising the amino acid sequence of SEQ ID NO: 95.

An antibody or a fragment thereof comprising a heavy-chain variable region comprising the amino acid sequence of SEQ ID NO: 96 and a light-chain variable region comprising the amino acid sequence of SEQ ID NO: 97.

An antibody or a fragment thereof comprising a heavy-chain variable region comprising the amino acid sequence of SEQ ID NO: 98 and a light-chain variable region comprising the amino acid sequence of SEQ ID NO: 99.

An antibody or a fragment thereof comprising a heavy-chain variable region comprising the amino acid sequence of SEQ ID NO: 100 and a light-chain variable region comprising the amino acid sequence of SEQ ID NO: 101.

An antibody or a fragment thereof comprising a heavy-chain variable region comprising the amino acid sequence of SEQ ID NO: 102 and a light-chain variable region comprising the amino acid sequence of SEQ ID NO: 103.

An antibody or a fragment thereof comprising a heavy-chain variable region comprising the amino acid sequence of SEQ ID NO: 104 and a light-chain variable region comprising the amino acid sequence of SEQ ID NO: 105.

An antibody or a fragment thereof comprising a heavy-chain variable region comprising the amino acid sequence of SEQ ID NO: 106 and a light-chain variable region comprising the amino acid sequence of SEQ ID NO: 107.

An antibody or a fragment thereof comprising a heavy-chain variable region comprising the amino acid sequence of SEQ ID NO: 108 and a light-chain variable region comprising the amino acid sequence of SEQ ID NO: 109.

An antibody or a fragment thereof comprising a heavy-chain variable region comprising the amino acid sequence of SEQ ID NO: 110 and a light-chain variable region comprising the amino acid sequence of SEQ ID NO: 111.

An antibody or a fragment thereof comprising a heavy-chain variable region comprising the amino acid sequence of SEQ ID NO: 112 and a light-chain variable region comprising the amino acid sequence of SEQ ID NO: 113.

An antibody or a fragment thereof comprising a heavy-chain variable region comprising the amino acid sequence of SEQ ID NO: 114 and a light-chain variable region comprising the amino acid sequence of SEQ ID NO: 115.

An antibody or a fragment thereof comprising a heavy-chain variable region comprising the amino acid sequence of SEQ ID NO: 116 and a light-chain variable region comprising the amino acid sequence of SEQ ID NO: 117.

An antibody or a fragment thereof comprising a heavy-chain variable region comprising the amino acid sequence of SEQ ID NO: 118 and a light-chain variable region comprising the amino acid sequence of SEQ ID NO: 119.

An antibody or a fragment thereof comprising a heavy-chain variable region comprising the amino acid sequence of SEQ ID NO: 120 and a light-chain variable region comprising the amino acid sequence of SEQ ID NO: 121.

An antibody or a fragment thereof comprising a heavy-chain variable region comprising the amino acid sequence of SEQ ID NO: 122 and a light-chain variable region comprising the amino acid sequence of SEQ ID NO: 123.

An antibody or a fragment thereof comprising a heavy-chain variable region comprising the amino acid sequence of SEQ ID NO: 124 and a light-chain variable region comprising the amino acid sequence of SEQ ID NO: 125.

An antibody or a fragment thereof comprising a heavy-chain variable region comprising the amino acid sequence of SEQ ID NO: 126 and a light-chain variable region comprising the amino acid sequence of SEQ ID NO: 127.

An antibody or a fragment thereof comprising a heavy-chain variable region comprising the amino acid sequence of SEQ ID NO: 128 and a light-chain variable region comprising the amino acid sequence of SEQ ID NO: 129.

An antibody or a fragment thereof comprising a heavy-chain variable region comprising the amino acid sequence of SEQ ID NO: 130 and a light-chain variable region comprising the amino acid sequence of SEQ ID NO: 131.

An antibody or a fragment thereof comprising a heavy-chain variable region comprising the amino acid sequence of SEQ ID NO: 132 and a light-chain variable region comprising the amino acid sequence of SEQ ID NO: 133.

An antibody or a fragment thereof comprising a heavy-chain variable region comprising the amino acid sequence of SEQ ID NO: 300 and a light-chain variable region comprising the amino acid sequence of SEQ ID NO: 304.

In Examples mentioned later, the polyclonal antibody or the monoclonal antibody against a full-length CAPRIN-1 protein or a polypeptide of a portion of a region expressed on cell membrane surfaces of cancer cells was confirmed to exhibit reactivity with cell membrane surfaces of a plurality of human cancer cells. Furthermore, results indicating the response in human cancer patients were obtained, and a marked antitumor effect was obtained by which a tumor completely disappeared at some cancer sites.

### <Inhibitor of Synthesis of N-Glycoside-Linked Sugar Chain of Glycoprotein>

The inhibitor of synthesis of N-glycoside-linked sugar chain of a glycoprotein refers to a drug having the action of inhibiting a process of synthesis and processing of N-glycoside-linked sugar chain modification described in detail below, or a drug inhibiting the activity of any enzyme that works as a catalyst during the process.

Membrane proteins or secretory proteins synthesized in cells are finally transported onto the cell membranes or to the outside of the cells via endoplasmic reticulum and Golgi body. During this process, N-glycoside-linked sugar chain modifications are synthesized on amide groups of some asparagine (Asn) residues of the proteins, and O-glycoside-linked sugar chain modifications are synthesized on hydroxy groups of some serine (Ser)/threonine (Thr) residues of the proteins, in the endoplasmic reticulum and the Golgi body. The synthesis of the N-glycoside-linked sugar chain modification, among the above sugar chain modifications of glycoproteins, is started in the endoplasmic reticulum, and final sugar chain structures are completed through processing in the endoplasmic reticulum and the Golgi body. The final sugar chain structures are classified into three types (high-mannose, hybrid, and complex types). These three types of N-glycoside-linked sugar chain modifications differ from each other in non-reducing terminal structure, though they have a common reducing terminal pentasaccharide (called core pentasaccharide, which consists of two N-acetylglucosamines (GlcNAc) and three biantennary mannoses in order from the reducing end) near an Asn residue.

Proteins on which N-glycoside-linked sugar chains are synthesized are not limited to particular proteins, and all membrane proteins or secretory proteins may be targets for the modification. The Asn residues that can be targets for the modification may be all Asn residues as long as their neighboring amino acid sequences satisfy a condition of Asn-X-Ser/Thr (X: any amino acid residue).

In the biosynthesis of N-glycoside-linked sugar chains of proteins in eukaryotic cells, a dolichol-linked sugar chain, in which a sugar chain and dolichol are linked through an ester bond via pyrophosphate, is used as a sugar chain donor substrate. The initial step of the biosynthesis of the dolichol-linked sugar chain is a reaction of transferring GlcNAc-1 phosphate from UDP-GlcNAc to dolichol phosphate, and this reaction is catalyzed by an enzyme called dolichyl-phosphate N-acetylglucosaminephosphotransferase (also known as DPAGT1 or GPT). Then, the biosynthesis of a full-length dolichol-linked sugar chain Glc3Man9GlcNAc2-PP-dolichol (Glc3: three glucoses, Man9: nine mannoses, GlcNAc2: two N-acetylglucosamines, PP: pyrophosphate) is completed through multiple reactions catalyzed by a series of enzymes present in the endoplasmic reticulum, i.e., ALG13, ALG14, ALG1, ALG2, ALG11, RFT1, ALG3, ALG9, ALG12, ALG6, ALG8, and ALG10. The sugar chain moiety (Glc3Man9GlcNAc2) of the full-length dolichol-linked sugar chain is transferred all together to an Asn residue of a protein by an OST (oligosaccharyltransferase) complex. Then, the N-glycoside-linked sugar chain bound to the protein further undergoes processing by glucosidase I/II and mannosidase I/II which are glycolytic enzymes present in the endoplasmic reticulum or the Golgi body, and glucosyltransferase (GlcT-1), N-acetylglucosaminyltransferase (GNT-I/II/III/IV/V), fucosyltransferase (FUT1 to FUT11), galactosyltransferase (GALT), and sialyltransferase (ST) which are glycosyltransferases. In this way, a final N-glycoside-linked sugar chain is completed.

Specific examples of the inhibitor of synthesis of N-glycoside-linked sugar chain of a glycoprotein include DPAGT1 inhibitors, glucosidase I/II inhibitors and mannosidase I/II inhibitors. Specific examples of the DPAGT1 inhibitors include tunicamycin, CPPB (capuramycin phenoxypiperidinylbenzylamide), APPB (aminouridyl phenoxylpiperidinylbenzylbutanamide) and their pharmaceutically acceptable (known) salts or (known) derivatives. Specific examples of the glucosidase I/II inhibitors include australine, castanospermine, conduritol B epoxide, bromoconduritol, deoxynojirimycin, bromoconduritol and their pharmaceutically acceptable (known) salts or (known) derivatives. Specific examples of the mannosidase I/II inhibitors include kifunensine, deoxymannojirimycin, mannostatin A, swaisonine and their pharmaceutically acceptable (known) salts or (known) derivatives. The inhibitor of synthesis of N-glycoside-linked sugar chain of a glycoprotein is preferably a DPAGT1 inhibitor, more preferably tunicamycin, CPPB, APPB or a pharmaceutically acceptable (known) salt or (known) derivative thereof, and further preferably tunicamycin or a pharmaceutically acceptable derivative thereof.

Tunicamycin is a mixture of homologous nucleoside antibiotics having the activity of inhibiting dolichyl-phosphate N-acetylglucosaminephosphotransferase (also known as DPAGT1 or GPT), and is produced from Streptomycetes of the genus Streptomyces (typically Streptomyces lysosuperficus), and its CAS number is represented by 11089-65-9. The homologs in tunicamycin have different molecular weights depending on linked fatty acid side chains, and these individual homologs can be distinguished as tunicamycin I to X. The term “tunicamycin” described herein refers to a mixture comprising these homologs having different molecular weights at any ratio.

### <Other Drugs>

The medicament of the present invention may comprise, as an active ingredient, an antitumor agent known in literatures, etc., in addition to the anti-CAPRIN-1 antibody and the inhibitor of synthesis of N-glycoside-linked sugar chain of a glycoprotein, as long as it does not inhibit the effects as the medicament of the present invention. Specific examples of known antitumor agents include, but are not particularly limited to, 5-fluorouracil, irinotecan, oxaliplatin, carboplatin, cisplatin, nedaplatin, gemcitabine, paclitaxel, nabpaclitaxel, imiquimod, immune checkpoint inhibitors, doxorubicin, daunorubicin, cyclophosphamide, methotrexate, thiotepa, busulfan, improsulfan, piposulfan, benzodopa, carboquone, meturedopa, uredopa, altretamine, triethylenemelamine, triethylenephosphoramide, triethilenethiophosphoramide, trimethylolomelamine, bullatacin, bullatacinone, camptothecin, bryostatin, callystatin, cryptophycin 1, cryptophycin 8, dolastatin, duocarmycin, eleutherobin, pancratistatin, sarcodictyin, spongistatin, chlorambucil, chloRNAphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, calicheamicin, dynemicin, clodronate, esperamicin, aclacinomycin, actinomycin, authramycin, azaserine, bleomycin, cactinomycin, carabicin, carminomycin, carzinophilin, chromomycin, dactinomycin, detorbicin, 6-diazo-5-oxo-L-norleucine, adriamycin, epirubicin, esorubicin, idarubicin, marcellomycin, mitomycin C, mycophenolic acid, nogalamycin, olivomycin, peplomycin, potfiromycin, puromycin, quelamycin, rodorubicin, streptonigrin, streptozocin, tubercidin, ubenimex, zinostatin, zorubicin, denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, epothilone, etoglucid, lentinan, lonidamine, maytansine, ansamitocine, mitoguazone, mitoxantrone, mopidanmol, nitraerine, pentostatin, phenamet, pirarubicin, losoxantrone, podophyllinic acid, 2-ethylhydrazide, procarbazine, razoxane, rhizoxin, schizophyllan, spirogermanium, tenuazonic acid, triaziquone, roridine A, anguidine, urethane, vindesine, dacarbazine, mannomustine, mitobronitol, mitolactol, pipobroman, gacytosine, docetaxel, chlorambucil, gemcitabine, 6-thioguanine, mercaptopurine, vinblastine, etoposide, ifosfamide, mitoxantrone, vincristine, vinorelbine, novantrone, teniposide, edatrexate, daunomycin, aminopterin, xeloda, ibandronate, difluoromethylornithine (DMFO), topoisomerase inhibitors, retinoic acid, folinic acid, and their pharmaceutically acceptable (known) salts or (known) derivatives.

### <Antitumor Effect of Present Invention>

The antitumor effect of the combination of the anti-CAPRIN-1 antibody and the inhibitor of synthesis of N-glycoside-linked sugar chain of a glycoprotein according to the present invention can be evaluated in vivo or in vitro. The in vivo antitumor effect can be evaluated by administering the anti-CAPRIN-1 antibody and the inhibitor of synthesis of N-glycoside-linked sugar chain of a glycoprotein to an organism having cancer, measuring the size of a tumor after the administration, and examining the size of the cancer over time. Also, the in vivo antitumor effect can be evaluated by examining a survival rate of organisms. Alternatively, the in vivo antitumor effect may be evaluated by examining the ability to produce cytokines or chemokines. The in vivo antitumor effect can be further evaluated by examining prevention of cancer, prevention of metastasis or prevention of recurrence.

The in vitro antitumor effect can be evaluated by examining the cytotoxic activity or phagocytic activity against cancer cells by immunocytes in coculturing the cancer cells and the immunocytes. Thus, the antitumor effect of the combination of the anti-CAPRIN-1 antibody and the inhibitor of synthesis of N-glycoside-linked sugar chain of a glycoprotein can be evaluated by adding the anti-CAPRIN-1 antibody and the inhibitor of synthesis of N-glycoside-linked sugar chain of a glycoprotein in combination to a coculture system of cancer cells and immunocytes, and examining the cytotoxic activity or phagocytic activity against the cancer cells by the immunocytes. The immunocyte for use here may be any cell as long as the immunocyte is a blood cell having cytotoxic activity or phagocytic activity. A human NK cell is preferred for evaluating the cytotoxic activity, and a human monocytic cell (THP-1 or U937) is preferred for evaluating the phagocytic activity. When an antibody binds to a cancer cell, this is recognized by an immunocyte so that the cancer cell is killed via cytotoxic activity or phagocytic activity by the immunocyte. Therefore, an in vivo antitumor effect can be predicted by evaluating the in vitro antitumor effect.

An ability of an anti-CAPRIN-1 antibody used in the present invention to bind to CAPRIN-1 can be determined via binding assay using, for example, ELISA, a Western blot method, immunofluorescence, or flowcytometry analysis.

The combination of the anti-CAPRIN-1 antibody and the inhibitor of synthesis of N-glycoside-linked sugar chain of a glycoprotein according to the present invention increases an in vitro antitumor effect as compared with the anti-CAPRIN-1 antibody alone. The rate of increase is preferably 1.5 or more times, more preferably 2 or more times, and further preferably 3 or more times.

<Medicament for Treatment and/or Prevention of Cancer>

A medicament of the present invention is aimed at treating and/or preventing cancer. A cancer targeted by the medicament of the present invention is not particularly limited as long as it is a cancer (cells) expressing CAPRIN-1 protein, particularly, a cancer (cells) expressing CAPRIN-1 protein on a cell membrane surface.

The term “treatment” used herein refers to treatment of cancer based on an antitumor effect mentioned above. The term “prevention” used herein refers to not only prevention of development of cancer but also prevention of metastasis or recurrence of cancer.

Both the terms “tumor” and “cancer” used herein refer to malignant neoplasm, and thus they are used in an exchangeable manner.

Cancer that can be a target in the present invention is any cancer as long as the cancer expresses CAPRIN-1 protein on a cell membrane surface. The cancer is preferably colon cancer, melanoma, lung cancer, prostate cancer, ovarian cancer, kidney cancer, breast cancer, gastric cancer, pancreatic cancer, bile duct cancer, renal cell cancer, Hodgkin's lymphoma, head and neck cancer, mesothelioma, colorectal cancer, esophageal cancer, gastroesophageal cancer, hepatocellular cancer, glioblastoma, urothelial cancer, urinary bladder cancer, uterus cancer, primary central nervous system lymphoma, primary testicular lymphoma, biliary tract cancer, brain tumor, leukemia, lymphoma, liver cancer, sarcoma, fibrosarcoma, mastocytoma, adrenal cortex cancer, Ewing's tumor, multiple myeloma, testicle cancer, thyroid cancer, basal cell cancer, Paget's disease or skin cancer. These cancers may be primary cancers, metastatic cancers, cancers that have metastasized, cancers that have recurred, cancers after surgery, or unresectable cancers. The term melanoma is often used interchangeably with malignant melanoma.

More specifically, examples of the cancer include, but are not limited to, for example, Bowen's disease, prickle cell cancer, extramammary Paget's disease, mycosis fungoides, Sezary's syndrome, cutaneous T/NK-cell lymphoma, T-cell leukemia or lymphoma having a lesion only in the skin, cutaneous B-cell lymphoma (indolent group), cutaneous T-cell lymphatic or mammary adenoma, complex mammary adenoma, malignant mixed tumor of mammary gland, intraductal papillary carcinoma of mammary gland, lung adenocarcinoma, squamous cell cancer, small cell cancer, large cell cancer, glioma which is a neuroepithelial tissue tumor, glioblastoma, neuroblastoma, ependymoma, neuronal tumor, neuroectodermal tumor, neurilemoma, neurofibromatosis, meningioma, chronic lymphocytic leukemia, lymphoma, alimentary lymphoma, gastrointestinal lymphoma, small to medium cell lymphoma, cecal cancer, ascending colon cancer, descending colon cancer, transverse colon cancer, sigmoid colon cancer, rectal cancer, ovarian epithelial cancer, germ cell tumor, interstitial cell tumor, pancreatic duct cancer, invasive pancreatic duct cancer, adenocarcinoma of pancreatic cancer, acinar cell cancer, adenosquamous cancer, giant cell tumor, intraductal papillary mucinous tumor, mucinous cystadenocarcinoma, pancreatoblastoma, pancreatic islet cell tumor, Frantz's tumor, serous cystadenocarcinoma, solid pseudopapillary cancer, gastrinoma, glucagonoma, insulinoma, multiple endocrine neoplasia type-1 (Wermer syndrome), nonfunctional islet cell tumor, somatostatinoma, VIPoma, uterine cervical cancer, uterine body cancer, fibrosarcoma, osteosarcoma, joint sarcoma, Ewing sarcoma, Wilms's tumor, hepatoblastoma, soft tissue sarcoma, acute leukemia, chronic leukemia, spinal cord tumor, malignant soft tissue tumor, tumors of teratoma group, and head and neck cancer including hypopharynx cancer, oropharynx cancer, tongue cancer, nasopharyngeal cancer, oral cavity cancer, lip cancer, sinus cancer, voice box cancer, cancer of the renal pelvis and ureter, urinary bladder cancer, urethra cancer, testicular tumor, malignant pleural mesothelioma, malignant bone tumor, uterine body cancer (postoperative chemotherapy, chemotherapy at the time of metastasis or relapse), and pediatric malignant solid tumor (rhabdomyosarcoma, neuroblastoma, hepatoblastoma, medulloblastoma, nephroblastoma, retinoblastoma, central nervous system germ cell tumor, and Ewing sarcoma family of tumors). The cancer also includes a palpable cancer, a subcutaneously existing cancer, an intracutaneously existing cancer, a superficial cancer, cancer existing in the dermis, cancer existing in a non-parenchymal organ, and progressive cancer which originate from the cancers described above. The cancer also includes a palpable cancer, a subcutaneously existing cancer, an intracutaneously existing cancer, a superficial cancer, cancer existing in the dermis, and cancer existing in a non-parenchymal organ, which originate from the cancers described above and have metastasized and recurred.

A preferable subject (patient) that can be a target is a mammal and is, for example, a mammal including primates, pet animals, livestock animals, and sport animals. Humans, dogs and cats are particularly preferable.

A medicament of the present invention can be formulated by a method known to persons skilled in the art. For instance, the medicament of the present invention can be parenterally used in the form of a parenteral injection of: an aseptic solution comprising water or a pharmacologically acceptable non-water solution; or a suspension liquid. For each formulation or pharmaceutical composition in the medicament of the present invention, the active ingredient (at least one of the anti-CAPRIN-1 antibody and the inhibitor of synthesis of N-glycoside-linked sugar chain of a glycoprotein) may be appropriately combined with, for example, a pharmacologically acceptable carrier, medium or additive; specifically, sterilized water, physiological saline, an isotonic solution, a buffer (buffer solution, etc.), plant oil, an oily solution, an antioxidant, a solubilizer, an emulsifier, a suspension, a surfactant, a stabilizer, a fragrance, an excipient, or a binder, and preferably, may be formulated by mixing with them in a unit dosage form required for a generally acceptable pharmaceutical formulation. An amount of an active ingredient in a formulation is determined such that an appropriate dosage within an indicated range can be achieved.

An aseptic composition for injection can be prepared in accordance with general formulation practice using a vehicle such as distilled water for injection. An aqueous solution for injection purposes includes, for example, physiological saline or isotonic solutions comprising glucose and other adjuvants such as D-sorbitol, D-mannose, D-mannitol, and sodium chloride. Such solution may be used with an appropriate dissolution aid. Such dissolution aid includes, for example, alcohols such as ethanol and polyalcohol, such as propylene glycol, polyethylene glycol, or nonion surfactants such as polysorbate 80™ and HCO-60. Oily liquid includes, for example, sesame oil or soybean oil. Such oily liquid may be used in combination with a dissolution aid such as benzyl benzoate or benzyl alcohol. In addition, it may be mixed with a buffering agent such as a phosphate buffer solution or a sodium acetate buffer solution, a soothing agent such as procaine hydrochloride, a stabilizer such as benzyl alcohol or phenol, or an antioxidant. In general, a formulated injection solution is introduced into an adequate ample.

The above pharmaceutical composition is orally or parenterally administered. Preferably, it is parenterally administered. Specifically, dosage forms include injectable agents, intranasally-administered agents, transpulmonarily-administered agents, and percutaneously-administered agents. For example, injectable agents can be systemically or locally administered via intravenous injection, intramuscular injection, intraperitoneal injection, subcutaneous injection, or intratumoral injection. The percutaneously-administered agents include, for example, agents called liniments and external medicines. The external medicines include, for example, solid agents, solutions, sprays, ointments, creams, and gels.

The administration method can be appropriately determined depending on age, weight, gender, and symptoms of a patient. A single dose of a pharmaceutical composition comprising at least one of the anti-CAPRIN-1 antibody and the inhibitor of synthesis of N-glycoside-linked sugar chain of a glycoprotein can be selected within a range of, for example, 0.0001 mg to 1000 mg per kg of body weight, as the amount of each active ingredient. Alternatively, the dose of each active ingredient can be selected within a range of, for example, 0.001 to 100000 mg per patient's body, or 0.1 mg to 300 mg or 1 mg to 30 mg per kg of patient's body weight; however, it is not necessarily limited thereto. The dose and the administration method are changed depending on patient age, weight, gender, and symptoms. However, persons skilled in the art can appropriately select the dose and the method.

### <Administration Method>

Treatment and/or prevention of cancer with a medicament for treatment and/or prevention of cancer of the present invention includes various modes, in addition to administration as a medicament mentioned above. For example, respective active ingredients in a medicament of the present invention can be administered simultaneously, concurrently, or individually in a staggered manner. As a specific example, active ingredients can be administered within a time interval up to approximately 3 weeks, i.e., the second active ingredient can be administered from immediately up to approximately 3 weeks after administration of the first active ingredient. These administrations may be carried out subsequently to a surgical procedure, or a surgical procedure may be carried out between the administrations of the first and second drugs. In addition, the medicament for treatment and/or prevention of cancer of the present invention may be administered according to a plurality of administration cycles. For example, in the case of carrying out simultaneous administration of respective active ingredients in a medicament for treatment and/or prevention of cancer of the present invention, a pharmaceutical composition comprising both active ingredients is administered once per approximately 2 days to approximately 3 weeks as one cycle. Then, this treatment cycle may be repeated, if necessary, according to the judgment of a physician in charge. Likewise, in the case of scheduling a formulation in a staggered manner, respective administration periods of individual agents are adjusted so as to span the same period. The interval between cycles can vary from 0 to 2 months. Respective doses of the active ingredients in the medicament for treatment and/or prevention of cancer of the present invention can be set in the same way as in the respective doses of the active ingredients in the pharmaceutical composition.

### <Pharmaceutical Kit>

A medicament for treatment and/or prevention of cancer of the present invention may be in the form of a pharmaceutical kit. The pharmaceutical kit is a package for using active ingredients in the form of separate pharmaceutical compositions (formulations) in a method for treating and/or preventing cancer. The package may comprise an instruction for administering each of the active ingredients. The respective active ingredients in the pharmaceutical compositions for treatment and/or prevention of cancer contained in the pharmaceutical kit can be in the form of pharmaceutical compositions each formulated as described above such that the active ingredients can be administered together or separately. Further, the pharmaceutical kit comprises active ingredients in amounts sufficient for one or more doses such that the active ingredients can be administered according to the administration method described above.

<Treatment and/or Prevention Method>

On the basis of the contents specifically described above, the present invention further provides a method for treating and/or preventing cancer, comprising administering the medicament of the present invention, or the anti-CAPRIN-1 antibody and the inhibitor of synthesis of N-glycoside-linked sugar chain of a glycoprotein according to the present invention to a subject (patient). For example, the present invention further provides a method for treating and/or preventing cancer, comprising administering the medicament, etc. of the present invention to a subject (patient) having cancer or suspected of having cancer. In this embodiment, for example, the anti-CAPRIN-1 antibody (antibody or fragment thereof) and the inhibitor of synthesis of N-glycoside-linked sugar chain of a glycoprotein according to the present invention, and an optional antitumor agent contained in the medicament can be administered simultaneously or separately to the subject (patient)

### EXAMPLES

The present invention is hereafter described in detail with reference to the following examples, although the scope of the present invention is not limited thereto.

### (Example 1) Production of Anti-CAPRIN-1 Antibody

One hundred (100) μg of a human CAPRIN-1 recombinant protein produced according to Example 3 of WO2010/016526 was mixed with a MPL+TDM adjuvant (Sigma) in an amount equivalent to that of the antigen protein. The mixture was used as an antigen solution per mouse. The antigen solution was administered intraperitoneally to 6-week-old Balb/c mice (Japan SLC Inc.) and then further administered 3 times and 24 times every week for completion of immunization. A spleen was removed on day 3 after the final immunization and then ground between two sterilized glass slides. Spleen cells were obtained by repeating the following procedure three times: washing with PBS (−), centrifuging at 1500 rpm for 10 minutes, and removing supernatant. The obtained spleen cells were mixed with mouse myeloma cell SP2/0 (purchased from ATCC) at a ratio of 10:1. The PEG solution prepared by mixing 200 μl of RPMI1640 medium containing 10% FBS heated at 37° C. and 800 μl of PEG1500 (Boehringer) was added to the cells. The solution was incubated for 5 minutes for cell fusion. Centrifugation was performed at 1700 rpm for 5 minutes to remove supernatants. Cells were suspended in 150 ml of RPMI1640 medium (HAT selective medium) containing 15% FBS, to which 2% equivalent of HAT solution (Gibco) had been added and then seeded onto fifteen 96-well plates (Nunc) at 100 μl per well. Cells were cultured for 7 days under conditions of 37° C. and 5% CO₂, so that hybridomas resulting from fusion of spleen cells to myeloma cells were obtained. Hybridomas were selected using binding affinity to CAPRIN-1 protein of the antibody produced by the prepared hybridomas as an indicator. The CAPRIN-1 protein solution (1 μg/ml) was added at 100 μl per well of 96-well plates and then incubated at 4° C. for 18 hours. After each well was washed 3 times with PBS-T, 0.5% Bovine Serum Albumin (BSA) solution (Sigma) was added at 400 μl per well, and then the plates were incubated at room temperature for 3 hours. The solution was removed and then each well was washed 3 times with 400 μl of PBS-T. Each culture supernatant of the hybridomas obtained above was added at 100 μl per well and then incubated at room temperature for 2 hours. After each well was washed 3 times with PBS-T, an HRP-labeled anti-mouse IgG (H+L) antibody (Invitrogen) diluted 5000-fold with PBS was added at 100 μl per well and then incubated at room temperature for 1 hour. After each well was washed 3 times with PBS-T, a TMB substrate solution (Thermo) was added at 100 μl per well and then incubated for 15-30 minutes, so that a color reaction was performed. After color development, 1 N sulfuric acid was added at 100 μl per well to stop the reaction. Absorbance at 450 nm and absorbance at 595 nm were measured using an absorption spectrometer. As a result, a plurality of hybridomas producing antibodies with high absorbances were selected. The selected hybridomas were added at 0.5 hybridomas per well of 96-well plates and then cultured. After 1 week, hybridomas forming single colonies in wells were observed. Cells in these wells were further cultured. Hybridomas were selected using binding affinity to CAPRIN-1 protein of the antibody produced by cloned hybridomas as an indicator. The CAPRIN-1 protein solution (1 μg/ml) was added at 100 μl per well of 96-well plates and then incubated at 4° C. for 18 hours. Each well was washed 3 times with PBS-T, a 0.5% BSA solution was added at 400 μl per well, and then incubated at room temperature for 3 hours. The solution was removed and then each well was washed 3 times with 400 μl of PBS-T. Each culture supernatant of the hybridomas obtained above was added at 100 μl per well and then incubated at room temperature for 2 hours. Each well was washed 3 times with PBS-T, an HRP-labeled anti-mouse IgG (H+L) antibody (Invitrogen) diluted 5000-fold with PBS was added at 100 μl per well and then incubated at room temperature for 1 hour. Each well was washed 3 times with PBS-T, a TMB substrate solution (Thermo) was added at 100 μl per well and then incubated for 15-30 minutes, so that a color reaction was performed. After color development, 1 N sulfuric acid was added at 100 μl per well to stop the reaction. Absorbance at 450 nm and absorbance at 595 nm were measured using an absorption spectrometer. As a result, a plurality of mouse monoclonal antibodies exerting reactivity with CAPRIN-1 protein were obtained.

Reactivity of each monoclonal antibody with human cancer cells confirmed to express CAPRIN-1 protein on cell membrane surfaces was further confirmed by flow cytometry. A mouse IgG control antibody exhibiting no reactivity with the cancer cells was used as a negative control. As a result of confirmation, several monoclonal antibodies were obtained which had stronger fluorescence intensity against the cancer cells than that of the mouse IgG control antibody and reacted strongly with the cell membrane surfaces of the cancer cells expressing CAPRIN-1 on the cell membrane surfaces. From among them, a monoclonal antibody against CAPRIN-1 described in WO2013/125630, which was an antibody comprising the amino acid sequence of a heavy-chain variable region shown by SEQ ID NO: 114 and the amino acid sequence of a light-chain variable region shown by SEQ ID NO: 115, was selected as a monoclonal antibody exhibiting reactivity with CAPRIN-1 protein.

CDR1 to CDR3 of the heavy-chain variable region of the antibody selected were identified. A nucleotide sequence was designed so as to be able to express a heavy-chain variable region having framework regions comprising a human antibody sequence. This nucleotide sequence was inserted to a vector for mammalian expression having an insert of a human IgG1 heavy-chain constant region. Likewise, CDR1 to CDR3 of the light-chain variable region were identified. A nucleotide sequence was designed so as to be able to express a light-chain variable region having framework regions comprising a human antibody sequence. This nucleotide sequence was inserted to a vector for mammalian expression having an insert of a human IgG1 light-chain constant region. These two recombinant expression vectors were introduced to mammalian cells according to a general method and then a culture supernatant containing humanized monoclonal antibody #1 (humanized antibody #1) against CAPRIN-1 was obtained.

The amino acid sequences of heavy chain CDR1, CDR2, and CDR3 of the humanized monoclonal antibody #1 are shown by SEQ ID NOs: 272, 273, and 274, respectively. The amino acid sequences of light chain CDR1, CDR2, and CDR3 of the humanized monoclonal antibody #1 are shown by SEQ ID NOs: 275, 276, and 277, respectively.

The obtained culture supernatant containing the obtained humanized anti-CAPRIN-1 monoclonal antibody #1 was purified using Hitrap Protein A Sepharose FF (GE Healthcare Bio-Sciences) according to a general method, replaced with PBS (−), and filtered through a 0.22 μm filter (Millipore) for preparation of the filtrate.

The specific reactivity of the anti-CAPRIN-1 antibody to CAPRIN-1 protein was detected and confirmed by ELISA using CAPRIN-1 protein immobilized on a plate.

The reactivity of the anti-CAPRIN-1 antibody with cancer cells without permeation treatment of cell membranes was examined by flow cytometry to confirm that a portion of CAPRIN-1 was expressed on cell membrane surfaces of cancer cells as shown in Examples given below.

It was confirmed by flow cytometry that, against all of breast cancer cells (BT-474), colon cancer cells (HT-29 and HCT116), lung cancer cells (QG56, H1650 and A549), gastric cancer cells (NCI-N87), uterus cancer cells (HEC-1-A), prostate cancer cells (22Rv1 and DU145), pancreatic cancer cells (Panc10.5), liver cancer cells (Hep3B), ovarian cancer cells (SKOV3), kidney cancer cells (Caki-2), brain tumor cells (U-87 MG), urinary bladder cancer cells (T24), esophageal cancer cells (OE33), leukemia cells (OCI-AML5), lymphoma cells (Ramos), gallbladder cancer cells (TGBC14TKB), fibrosarcoma cells (HT-1080), and melanoma cells (G-361 and A375), which are human cancer cells confirmed to express CAPRIN-1 gene, and mouse kidney cancer cells (Renca) and mouse breast cancer cells (4T1) confirmed to express CAPRIN-1 gene, the humanized antibody #1 had stronger fluorescence intensity than that of a human IgG control antibody and rabbit IgG antibody serving as negative controls exhibiting no reactivity with the cancer cells, and reacted strongly with the cell membrane surfaces of the cancer cells expressing CAPRIN-1.

Likewise, the anti-CAPRIN-1 antibodies described in WO2010/016526, WO2011/096517, WO2011/096528, WO2011/096519, WO2011/096533, WO2011/096534, WO2011/096535, WO2013/018886, WO2013/018894, WO2013/018892, WO2013/018891, WO2013/018889, WO2013/018883, WO2013/125636, WO2013/125654, WO2013/125640, WO2013/147169, WO2013/147176 and WO2015/020212 were also confirmed to react strongly with the cancer cell membrane surfaces.

### (Example 2) In Vitro Antitumor Effect of Combination of Anti-CAPRIN-1 Antibody and Tunicamycin

Antitumor effects of a combination of an anti-CAPRIN-1 antibody and tunicamycin, which is an inhibitor of synthesis of N-glycoside-linked sugar chain of a glycoprotein, were evaluated in vitro. Antitumor effects of combinations of an anti-CAPRIN-1 antibody and an existing chemotherapeutic fluorouracil (5-FU) or irinotecan (CPT-11) were evaluated simultaneously therewith and compared with the antitumor effects of the combination of the anti-CAPRIN-1 antibody and tunicamycin. The tunicamycin used was Tunicamycin Mixture (Item No. 11445) purchased from Cayman Chemical Company.

Specifically, in each combination test group (a tunicamycin combination test group, a fluorouracil combination test group, and an irinotecan combination test group), the indicated drug used in combination was applied to human cancer cells, and then the cancer cells were cocultured with human monocytic cells (THP-1) in the presence of the anti-CAPRIN-1 antibody, and the antibody-mediated phagocytic activity against the cancer cells by THP-1 was evaluated. The human-derived cancer cells used were a colon cancer cell line HCT116, a melanoma cell line A375 (used only in the tunicamycin combination test group), a lung cancer cell line A549 (used only in the tunicamycin combination test group), and a prostate cancer cell line DU145 (used only in the tunicamycin combination test group). On a 6-well plate, the human-derived cancer cells were cultured for 1 day in the presence of tunicamycin for the tunicamycin combination test group, and cultured for 2 days in the presence of the relevant drug used in combination for the fluorouracil combination test group and the irinotecan combination test group. The concentration of the drug used in combination was set to a concentration that decreased the proliferation rate of the cancer cells by about half at the completion of culture. Specifically, HCT116 was supplemented with tunicamycin at a concentration of 0.5 μM, fluorouracil at a concentration of 0.4 μg/mL, or irinotecan at a concentration of 15 μM; A375 was supplemented with tunicamycin at a concentration of 1 μM; A549 was supplemented with tunicamycin at a concentration of 5 μM; and DU145 was supplemented with tunicamycin at a concentration of 2 μM. As a control, a test group without a drug used in combination was established. In the control group, only DMSO (dimethyl sulfoxide), a solvent for the drug used in combination, was added to the cancer cells at a final concentration of 0.1% (v/v), and then the cancer cells were cultured for 2 days.

Each cancer cell line after the culture was dissociated with TrypLE Express (Thermo), and the cancer cells were stained by the addition of calcein-AM at a final concentration of 0.02 μg/mL and incubation at 37° C. for 30 minutes. Next, the cells were dispensed at 1×10⁴ cancer cells per well to a 96-well plate, and the anti-CAPRIN-1 antibody (final concentration: 5 μg/mL) and 1×10⁵ THP-1 cells were added to each well, followed by culture at 37° C. for 2.5 hours under a condition of 5% CO₂. Then, the cells were washed with PBS (phosphate buffer solution) containing 1% FBS (fetal bovine serum), and the human monocytic cells were stained through a reaction with an APC (allophycocyanin)-labeled anti-human CD45 antibody (final concentration: 0.25 μg/mL). Further, dead cells were stained with propidium iodide (PI) (final concentration: 0.1 μg/mL), followed by the measurement of fluorescence from the cells by flow cytometry. PI-positive dead cells were excluded from analysis. The human monocytic cells THP-1 recognize an antibody bound onto cancer cells and phagocytize the cancer cells. At that time, if the cancer cells have been stained with calcein-AM, the monocytic cells ingesting the cancer cells by phagocytosis also become positive to calcein-AM. Thus, the phagocytic activity in this evaluation system was calculated as the ratio (%) of APC-positive/calcein-AM-positive cells to the whole calcein-AM-positive population.

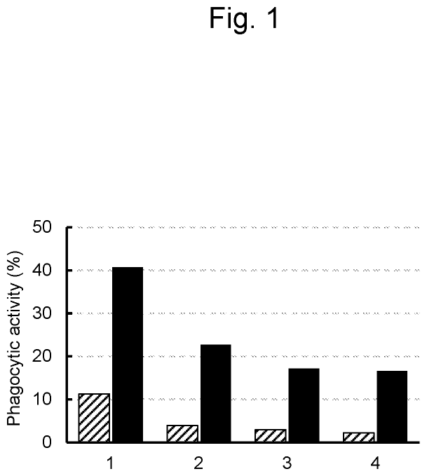

As a result of evaluation using the anti-CAPRIN-1 antibody prepared in Example 1 (anti-CAPRIN-1 humanized antibody #1) as an anti-CAPRIN-1 antibody, phagocytic activity against cancer cells of 11%, 4%, 3%, and 2% was observed for HCT116, A375, A549, and DU145, respectively, in the test group without a drug used in combination, whereas phagocytic activity against cancer cells of 41%, 23%, 17%, and 17% was observed for HCT116, A375, A549, and DU145, respectively, in the tunicamycin combination test group. Thus, the antitumor effect of the anti-CAPRIN-1 antibody on these four human cancer cells was increased by approximately 3 to 8 times by using the anti-CAPRIN-1 antibody in combination with tunicamycin (FIG. 1).

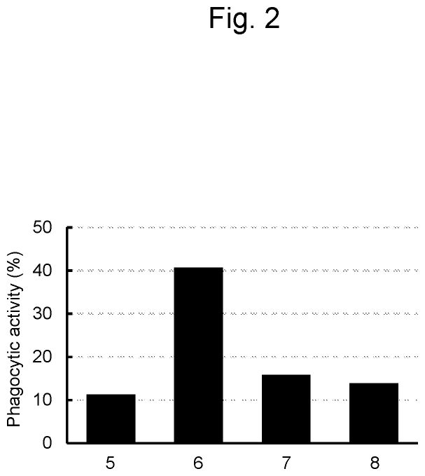

For HCT116, phagocytic activity against cancer cells was 41% in the tunicamycin combination test group, whereas the phagocytic activity against cancer cells was 16% and 14% in the fluorouracil combination test group and the irinotecan combination test group, respectively. Thus, the combination of the anti-CAPRIN-1 antibody and tunicamycin strongly increased the phagocytic activity against cancer cells as compared with the combinations of the anti-CAPRIN-1 antibody and the existing chemotherapeutics (FIG. 2).

Combinations of any anti-CAPRIN-1 antibody described in WO2010/016526, WO2011/096517, WO2011/096528, WO2011/096519, WO2011/096533, WO2011/096534, WO2011/096535, WO2013/018886, WO2013/018894, WO2013/018892, WO2013/018891, WO2013/018889, WO2013/018883, WO2013/125636, WO2013/125654, WO2013/125640, WO2013/147169, WO2013/147176 and WO2015/020212 and tunicamycin exhibit similar increase in phagocytic activity against the above human cancer cells as in the combination of the anti-CAPRIN-1 humanized antibody #1 and tunicamycin.

All publications, patents and patent applications cited herein are incorporated herein by reference in their entirety.

1. A medicament for treatment and/or prevention of cancer, comprising an antibody or a fragment thereof having an immunological reactivity with CAPRIN-1 protein, and an inhibitor of synthesis of N-glycoside-linked sugar chain of a glycoprotein together or separately in combination.

2. The medicament according to claim 1, wherein the inhibitor of synthesis of N-glycoside-linked sugar chain of a glycoprotein is tunicamycin or a pharmaceutically acceptable derivative thereof.

3. The medicament according to claim 1, wherein the antibody or the fragment thereof has an immunological reactivity with CAPRIN-1 protein having an amino acid sequence shown by any one of the even numbered SEQ ID NOs: 2 to 30, or an amino acid sequence having 80% or more sequence identity with the amino acid sequence.

4. The medicament according to claim 1, wherein the antibody or the fragment thereof has an immunological reactivity with an extracellular region of CAPRIN-1 protein present on a cancer cell surface.

5. The medicament according to claim 1, wherein the antibody or the fragment thereof has an immunological reactivity with a partial polypeptide of CAPRIN-1 protein, the partial polypeptide having an amino acid sequence shown by any one of SEQ ID NOs: 31 to 35, 296 to 299, 308 and 309, or an amino acid sequence having 80% or more sequence identity with the amino acid sequence.

6. The medicament according to claim 1, wherein the antibody is a monoclonal antibody or a polyclonal antibody.

7. The medicament according to claim 1, wherein the antibody or the fragment thereof is any one of the following (A) to (M):
(A) an antibody or a fragment thereof comprising a heavy-chain variable region comprising complementarity determining regions of SEQ ID NOs: 36, 37 and 38 (CDR1, CDR2 and CDR3, respectively) and a light-chain variable region comprising complementarity determining regions of SEQ ID NOs: 40, 41 and 42 (CDR1, CDR2 and CDR3, respectively), and having an immunological reactivity with CAPRIN-1 protein;
(B) an antibody or a fragment thereof comprising a heavy-chain variable region comprising complementarity determining regions of SEQ ID NOs: 44, 45 and 46 (CDR1, CDR2 and CDR3, respectively) and a light-chain variable region comprising complementarity determining regions of SEQ ID NOs: 48, 49 and 50 (CDR1, CDR2 and CDR3, respectively), and having an immunological reactivity with CAPRIN-1 protein;
(C) an antibody or a fragment thereof comprising a heavy-chain variable region comprising complementarity determining regions of SEQ ID NOs: 52, 53 and 54 (CDR1, CDR2 and CDR3, respectively) and a light-chain variable region comprising complementarity determining regions of SEQ ID NOs: 56, 57 and 58 (CDR1, CDR2 and CDR3, respectively), and having an immunological reactivity with CAPRIN-1 protein;
(D) an antibody or a fragment thereof comprising a heavy-chain variable region comprising complementarity determining regions of SEQ ID NOs: 60, 61 and 62 (CDR1, CDR2 and CDR3, respectively) and a light-chain variable region comprising complementarity determining regions of SEQ ID NOs: 64, 65 and 66 (CDR1, CDR2 and CDR3, respectively), and having an immunological reactivity with CAPRIN-1 protein;
(E) an antibody or a fragment thereof comprising a heavy-chain variable region comprising complementarity determining regions of SEQ ID NOs: 170, 171 and 172 (CDR1, CDR2 and CDR3, respectively) and a light-chain variable region comprising complementarity determining regions of SEQ ID NOs: 173, 174 and 175 (CDR1, CDR2 and CDR3, respectively), and having an immunological reactivity with CAPRIN-1 protein;
(F) an antibody or a fragment thereof comprising a heavy-chain variable region comprising complementarity determining regions of SEQ ID NOs: 176, 177 and 178 (CDR1, CDR2 and CDR3, respectively) and a light-chain variable region comprising complementarity determining regions of SEQ ID NOs: 179, 180 and 181 (CDR1, CDR2 and CDR3, respectively), and having an immunological reactivity with CAPRIN-1 protein;
(G) an antibody or a fragment thereof comprising a heavy-chain variable region comprising complementarity determining regions of SEQ ID NOs: 182, 183 and 184 (CDR1, CDR2 and CDR3, respectively) and a light-chain variable region comprising complementarity determining regions of SEQ ID NOs: 185, 186 and 187 (CDR1, CDR2 and CDR3, respectively), and having an immunological reactivity with CAPRIN-1 protein;
(H) an antibody or a fragment thereof comprising a heavy-chain variable region comprising complementarity determining regions of SEQ ID NOs: 188, 189 and 190 (CDR1, CDR2 and CDR3, respectively) and a light-chain variable region comprising complementarity determining regions of SEQ ID NOs: 191, 192 and 193 (CDR1, CDR2 and CDR3, respectively), and having an immunological reactivity with CAPRIN-1 protein;
(I) an antibody or a fragment thereof comprising a heavy-chain variable region comprising complementarity determining regions of SEQ ID NOs: 146, 147 and 148 (CDR1, CDR2 and CDR3, respectively) and a light-chain variable region comprising complementarity determining regions of SEQ ID NOs: 149, 150 and 151 (CDR1, CDR2 and CDR3, respectively), and having an immunological reactivity with CAPRIN-1 protein;
(J) an antibody or a fragment thereof comprising a heavy-chain variable region comprising complementarity determining regions of SEQ ID NOs: 272, 273 and 274 (CDR1, CDR2 and CDR3, respectively) and a light-chain variable region comprising complementarity determining regions of SEQ ID NOs: 275, 276 and 277 (CDR1, CDR2 and CDR3, respectively), and having an immunological reactivity with CAPRIN-1 protein;
(K) an antibody or a fragment thereof comprising a heavy-chain variable region comprising complementarity determining regions of SEQ ID NOs: 290, 291 and 292 (CDR1, CDR2 and CDR3, respectively) and a light-chain variable region comprising complementarity determining regions of SEQ ID NOs: 293, 294 and 295 (CDR1, CDR2 and CDR3, respectively), and having an immunological reactivity with CAPRIN-1 protein;
(L) an antibody or a fragment thereof comprising a heavy-chain variable region comprising complementarity determining regions of SEQ ID NOs: 301, 302 and 303 (CDR1, CDR2 and CDR3, respectively) and a light-chain variable region comprising complementarity determining regions of SEQ ID NOs: 305, 306 and 307 (CDR1, CDR2 and CDR3, respectively), and having an immunological reactivity with CAPRIN-1 protein; and
(M) an antibody or a fragment thereof comprising a heavy-chain variable region comprising complementarity determining regions of SEQ ID NOs: 134, 135 and 136 (CDR1, CDR2 and CDR3, respectively) and a light-chain variable region comprising complementarity determining regions of SEQ ID NOs: 137, 138 and 139 (CDR1, CDR2 and CDR3, respectively), and having an immunological reactivity with CAPRIN-1 protein.

8. The medicament according to claim 1, wherein the antibody or the fragment thereof is any one of the following (a) to (al):
(a) an antibody or a fragment thereof comprising a heavy-chain variable region comprising the amino acid sequence of SEQ ID NO: 39 and a light-chain variable region comprising the amino acid sequence of SEQ ID NO: 43;
(b) an antibody or a fragment thereof comprising a heavy-chain variable region comprising the amino acid sequence of SEQ ID NO: 47 and a light-chain variable region comprising the amino acid sequence of SEQ ID NO: 51;
(c) an antibody or a fragment thereof comprising a heavy-chain variable region comprising the amino acid sequence of SEQ ID NO: 55 and a light-chain variable region comprising the amino acid sequence of SEQ ID NO: 59;
(d) an antibody or a fragment thereof comprising a heavy-chain variable region comprising the amino acid sequence of SEQ ID NO: 63 and a light-chain variable region comprising the amino acid sequence of SEQ ID NO: 67;
(e) an antibody or a fragment thereof comprising a heavy-chain variable region comprising the amino acid sequence of SEQ ID NO: 68 and a light-chain variable region comprising the amino acid sequence of SEQ ID NO: 69;
(f) an antibody or a fragment thereof comprising a heavy-chain variable region comprising the amino acid sequence of SEQ ID NO: 70 and a light-chain variable region comprising the amino acid sequence of SEQ ID NO: 71;
(g) an antibody or a fragment thereof comprising a heavy-chain variable region comprising the amino acid sequence of SEQ ID NO: 72 and a light-chain variable region comprising the amino acid sequence of SEQ ID NO: 73;
(h) an antibody or a fragment thereof comprising a heavy-chain variable region comprising the amino acid sequence of SEQ ID NO: 74 and a light-chain variable region comprising the amino acid sequence of SEQ ID NO: 75;
(i) an antibody or a fragment thereof comprising a heavy-chain variable region comprising the amino acid sequence of SEQ ID NO: 76 and a light-chain variable region comprising the amino acid sequence of SEQ ID NO: 77;
(j) an antibody or a fragment thereof comprising a heavy-chain variable region comprising the amino acid sequence of SEQ ID NO: 78 and a light-chain variable region comprising the amino acid sequence of SEQ ID NO: 79;
(k) an antibody or a fragment thereof comprising a heavy-chain variable region comprising the amino acid sequence of SEQ ID NO: 80 and a light-chain variable region comprising the amino acid sequence of SEQ ID NO: 81;
(l) an antibody or a fragment thereof comprising a heavy-chain variable region comprising the amino acid sequence of SEQ ID NO: 82 and a light-chain variable region comprising the amino acid sequence of SEQ ID NO: 83;
(m) an antibody or a fragment thereof comprising a heavy-chain variable region comprising the amino acid sequence of SEQ ID NO: 84 and a light-chain variable region comprising the amino acid sequence of SEQ ID NO: 85;
(n) an antibody or a fragment thereof comprising a heavy-chain variable region comprising the amino acid sequence of SEQ ID NO: 86 and a light-chain variable region comprising the amino acid sequence of SEQ ID NO: 87;
(o) an antibody or a fragment thereof comprising a heavy-chain variable region comprising the amino acid sequence of SEQ ID NO: 88 and a light-chain variable region comprising the amino acid sequence of SEQ ID NO: 89;
(p) an antibody or a fragment thereof comprising a heavy-chain variable region comprising the amino acid sequence of SEQ ID NO: 90 and a light-chain variable region comprising the amino acid sequence of SEQ ID NO: 91;
(q) an antibody or a fragment thereof comprising a heavy-chain variable region comprising the amino acid sequence of SEQ ID NO: 92 and a light-chain variable region comprising the amino acid sequence of SEQ ID NO: 93;
(r) an antibody or a fragment thereof comprising a heavy-chain variable region comprising the amino acid sequence of SEQ ID NO: 94 and a light-chain variable region comprising the amino acid sequence of SEQ ID NO: 95;
(s) an antibody or a fragment thereof comprising a heavy-chain variable region comprising the amino acid sequence of SEQ ID NO: 96 and a light-chain variable region comprising the amino acid sequence of SEQ ID NO: 97;
(t) an antibody or a fragment thereof comprising a heavy-chain variable region comprising the amino acid sequence of SEQ ID NO: 98 and a light-chain variable region comprising the amino acid sequence of SEQ ID NO: 99;
(u) an antibody or a fragment thereof comprising a heavy-chain variable region comprising the amino acid sequence of SEQ ID NO: 100 and a light-chain variable region comprising the amino acid sequence of SEQ ID NO: 101;
(v) an antibody or a fragment thereof comprising a heavy-chain variable region comprising the amino acid sequence of SEQ ID NO: 102 and a light-chain variable region comprising the amino acid sequence of SEQ ID NO: 103;
(w) an antibody or a fragment thereof comprising a heavy-chain variable region comprising the amino acid sequence of SEQ ID NO: 104 and a light-chain variable region comprising the amino acid sequence of SEQ ID NO: 105;
(x) an antibody or a fragment thereof comprising a heavy-chain variable region comprising the amino acid sequence of SEQ ID NO: 106 and a light-chain variable region comprising the amino acid sequence of SEQ ID NO: 107;
(y) an antibody or a fragment thereof comprising a heavy-chain variable region comprising the amino acid sequence of SEQ ID NO: 108 and a light-chain variable region comprising the amino acid sequence of SEQ ID NO: 109;
(z) an antibody or a fragment thereof comprising a heavy-chain variable region comprising the amino acid sequence of SEQ ID NO: 110 and a light-chain variable region comprising the amino acid sequence of SEQ ID NO: 111;
(aa) an antibody or a fragment thereof comprising a heavy-chain variable region comprising the amino acid sequence of SEQ ID NO: 112 and a light-chain variable region comprising the amino acid sequence of SEQ ID NO: 113;
(ab) an antibody or a fragment thereof comprising a heavy-chain variable region comprising the amino acid sequence of SEQ ID NO: 114 and a light-chain variable region comprising the amino acid sequence of SEQ ID NO: 115;
(ac) an antibody or a fragment thereof comprising a heavy-chain variable region comprising the amino acid sequence of SEQ ID NO: 116 and a light-chain variable region comprising the amino acid sequence of SEQ ID NO: 117;
(ad) an antibody or a fragment thereof comprising a heavy-chain variable region comprising the amino acid sequence of SEQ ID NO: 118 and a light-chain variable region comprising the amino acid sequence of SEQ ID NO: 119;
(ae) an antibody or a fragment thereof comprising a heavy-chain variable region comprising the amino acid sequence of SEQ ID NO: 120 and a light-chain variable region comprising the amino acid sequence of SEQ ID NO: 121;
(af) an antibody or a fragment thereof comprising a heavy-chain variable region comprising the amino acid sequence of SEQ ID NO: 122 and a light-chain variable region comprising the amino acid sequence of SEQ ID NO: 123;
(ag) an antibody or a fragment thereof comprising a heavy-chain variable region comprising the amino acid sequence of SEQ ID NO: 124 and a light-chain variable region comprising the amino acid sequence of SEQ ID NO: 125;
(ah) an antibody or a fragment thereof comprising a heavy-chain variable region comprising the amino acid sequence of SEQ ID NO: 126 and a light-chain variable region comprising the amino acid sequence of SEQ ID NO: 127;
(ai) an antibody or a fragment thereof comprising a heavy-chain variable region comprising the amino acid sequence of SEQ ID NO: 128 and a light-chain variable region comprising the amino acid sequence of SEQ ID NO: 129;
(aj) an antibody or a fragment thereof comprising a heavy-chain variable region comprising the amino acid sequence of SEQ ID NO: 130 and a light-chain variable region comprising the amino acid sequence of SEQ ID NO: 131;
(ak) an antibody or a fragment thereof comprising a heavy-chain variable region comprising the amino acid sequence of SEQ ID NO: 132 and a light-chain variable region comprising the amino acid sequence of SEQ ID NO: 133; and
(al) an antibody or a fragment thereof comprising a heavy-chain variable region comprising the amino acid sequence of SEQ ID NO: 300 and a light-chain variable region comprising the amino acid sequence of SEQ ID NO: 304.

9. The medicament according to claim 1, wherein the antibody is a human antibody, a humanized antibody, a chimeric antibody or a single chain antibody.

10. The medicament according to claim 1, wherein the cancer is a cancer expressing CAPRIN-1 protein on a cell membrane surface.

11. The medicament according to claim 1, wherein the cancer is colon cancer, melanoma, lung cancer, prostate cancer, ovarian cancer, kidney cancer, breast cancer, gastric cancer, pancreatic cancer, bile duct cancer, renal cell cancer, Hodgkin's lymphoma, head and neck cancer, mesothelioma, colorectal cancer, esophageal cancer, gastroesophageal cancer, hepatocellular cancer, glioblastoma, urothelial cancer, urinary bladder cancer, uterus cancer, primary central nervous system lymphoma, primary testicular lymphoma, biliary tract cancer, brain tumor, leukemia, lymphoma, liver cancer, sarcoma, fibrosarcoma, mastocytoma, adrenal cortex cancer, Ewing's tumor, multiple myeloma, testicle cancer, thyroid cancer, basal cell cancer, Paget's disease or skin cancer.

12. An agent increasing drug efficacy of a pharmaceutical composition for treatment and/or prevention of cancer comprising an antibody or a fragment thereof having an immunological reactivity with CAPRIN-1 protein as an active ingredient, wherein the agent comprises an inhibitor of synthesis of N-glycoside-linked sugar chain of a glycoprotein as an active ingredient.

13. An agent increasing drug efficacy of a pharmaceutical composition for treatment and/or prevention of cancer comprising an inhibitor of synthesis of N-glycoside-linked sugar chain of a glycoprotein as an active ingredient, wherein the agent comprises an antibody or a fragment thereof having an immunological reactivity with CAPRIN-1 protein as an active ingredient.

14. A method for treating and/or preventing cancer, comprising administering an antibody or a fragment thereof having an immunological reactivity with CAPRIN-1 protein, and an inhibitor of synthesis of N-glycoside-linked sugar chain of a glycoprotein together or separately to a subject.


SEQUENCE LISTING (324 sequences)

SEQ ID NO: 1 - DNA, 5562 bp, Homo sapiens
  source 1..5562: mol_type = other DNA
  CDS 190..2319: protein_id = 310; translation = MPSATSHSGSGSKSSGPPPPSGSSGSEAAAGAGAAAPASQHPAT GTGAVQTEAMKQILGVIDKKLRNLEKKKGKLDDYQERMNKGERLNQDQLDAVSKYQEV TNNLEFAKELQRSFMALSQDIQKTIKKTARREQLMREEAEQKRLKTVLELQYVLDKLG DDEVRTDLKQGLNGVPILSEEELSLLDEFYKLVDPERDMSLRLNEQYEHASIHLWDLL EGKEKPVCGTTYKVLKEIVERVFQSNYFDSTHNHQNGLCEEEEAASAPAVEDQVPEAE PEPAEEYTEQSEVESTEYVNRQFMAETQFTSGEKEQVDEWTVETVEVVNSLQQQPQAA SPSVPEPHSLTPVAQADPLVRRQRVQDLMAQMQGPYNFIQDSMLDFENQTLDPAIVSA QPMNPTQNMDMPQLVCPPVHSESRLAQPNQVPVQPEATQVPLVSSTSEGYTASQPLYQ PSHATEQRPQKEPIDQIQATISLNTDQTTASSSLPAASQPQVFQAGTSKPLHSSGINV NAAPFQSMQTVFNMNAPVPPVNEPETLKQQNQYQASYNQSFSSQPHQVEQTELQQEQL QTVVGTYHGSPDQSHQVTGNHQQPPQQNTGFPRSNQPYYNSRGVSRGGSRGARGLMNG YRGPANGFRGGYDGYRPSFSNTPNSGYTQSQFSAPRDYSGYQRDGYQQNFKRGSGQSG PRGAPRGRGGPPRPNRGMPQMNTQQVN
cagagggctgctggctggctaagtccctcccgctcccggctctcgcctcactaggagcggctctcggtgcagcgggacagggcgaagcggcctgcgcccacggagcgcgcgacactgcccggaagggaccgccacccttgccccctcagctgcccactcgtgatttccagcggcctccgcgcgcgcacgatgccctcggccaccagccacagcgggagcggcagcaagtcgtccggaccgccaccgccgtcgggttcctccgggagtgaggcggccgcgggagccggggccgccgcgccggcttctcagcaccccgcaaccggcaccggcgctgtccagaccgaggccatgaagcagattctcggggtgatcgacaagaaacttcggaacctggagaagaaaaagggtaagcttgatgattaccaggaacgaatgaacaaaggggaaaggcttaatcaagatcagctggatgccgtttctaagtaccaggaagtcacaaataatttggagtttgcaaaagaattacagaggagtttcatggcactaagtcaagatattcagaaaacaataaagaagacagcacgtcgggagcagcttatgagagaagaagctgaacagaaacgtttaaaaactgtacttgagctacagtatgttttggacaaattgggagatgatgaagtgcggactgacctgaaacaaggtttgaatggagtgccaatattgtccgaagaggagttgtcattgttggatgaattctataagctagtagaccctgaacgggacatgagcttgaggttgaatgaacagtatgaacatgcctccattcacctgtgggacctgctggaagggaaggaaaaacctgtatgtggaaccacctataaagttctaaaggaaattgttgagcgtgtttttcagtcaaactactttgacagcacccacaaccaccagaatgggctgtgtgaggaagaagaggcagcctcagcacctgcagttgaagaccaggtacctgaagctgaacctgagccagcagaagagtacactgagcaaagtgaagttgaatcaacagagtatgtaaatagacagttcatggcagaaacacagttcaccagtggtgaaaaggagcaggtagatgagtggacagttgaaacggttgaggtggtaaattcactccagcagcaacctcaggctgcatccccttcagtaccagagccccactctttgactccagtggctcaggcagatccccttgtgagaagacagcgagtacaagaccttatggcacaaatgcagggtccctataatttcatacaggattcaatgctggattttgaaaatcagacacttgatcctgccattgtatctgcacagcctatgaatccaacacaaaacatggacatgccccagctggtttgccctccagttcattctgaatctagacttgctcagcctaatcaagttcctgtacaaccagaagcgacacaggttcctttggtatcatccacaagtgaggggtacacagcatctcaacccttgtaccagccttctcatgctacagagcaacgaccacagaaggaaccaattgatcagattcaggcaacaatctctttaaatacagaccagactacagcatcatcatcccttcctgctgcgtctcagcctcaagtatttcaggctgggacaagcaaacctttacatagcagtggaatcaatgtaaatgcagctccattccaatccatgcaaacggtgttcaatatgaatgccccagttcctcctgttaatgaaccagaaactttaaaacagcaaaatcagtaccaggccagttataaccagagcttttctagtcagcctcaccaagtagaacaaacagagcttcagcaagaacagcttcaaacagtggttggcacttaccatggttccccagaccagtcccatcaagtgactggtaaccaccagcagcctcctcagcagaacactggatttccacgtagcaatcagccctattacaatagtcgtggtgtgtctcgtggaggctcccgtggtgctagaggcttgatgaatggataccggggccctgccaatggattcagaggaggatatgatggttaccgcccttcattctctaacactccaaacagtggttatacacagtctcagttcagtgctccccgggattactctggctatcaacgggatggatatcagcagaatttcaagcgaggctctgggcagagtggaccacggggagccccacgaggtcgtggagggcccccaagacccaacagagggatgccgcaaatgaacactcagcaagtgaattaatctgattcacaggattatgtttaatcgccaaaaacacactggccagtgtaccataatatgttaccagaagagttattatctatttgttctccctttcaggaaacttattgtaaagggactgttttcatcccataaagacaggactacaattgtcagctttctattacctggatatggaaggaaactatttttactctgcatgttctgtcctaagcgtcatcttgagccttgcacatgatactcagattcctcacccttgcttaggagtaaaacaatatactttacagggtgataataatctccatagttatttgaagtggcttgaaaaaggcaagattgacttttatgacattggataaaatctacaaatcagccctcgagttattcaatgataactgacaaactaaattatttccctagaaaggaagatgaaaggagtggagtgtggtttggcagaacaactgcatttcacagcttttccagttaaattggagcactgaacgttcagatgcataccaaattatgcatgggtcctaatcacacatataaggctggctaccagctttgacacagcactgttcatctggccaaacaactgtggttaaaaacacatgtaaaatgctttttaacagctgatactgtataagacaaagccaagatgcaaaattaggctttgattggcactttttgaaaaatatgcaacaaatatgggatgtaatccggatggccgcttctgtacttaatgtgaaatatttagatacctttttgaacacttaacagtttctttgagacaatgacttttgtaaggattggtactatctatcattccttatgacatgtacattgtctgtcactaatccttggattttgctgtattgtcacctaaattggtacaggtactgatgaaaatctctagtggataatcataacactctcggtcacatgtttttccttcagcttgaaagcttttttttaaaaggaaaagataccaaatgcctgctgctaccacccttttcaattgctatcttttgaaaggcaccagtatgtgttttagattgatttccctgtttcagggaaatcacggacagtagtttcagttctgatggtataagcaaaacaaataaaacgtttataaaagttgtatcttgaaacactggtgttcaacagctagcagcttatgtgattcaccccatgccacgttagtgtcacaaattttatggtttatctccagcaacatttctctagtacttgcacttattatcttttgtctaatttaaccttaactgaattctccgtttctcctggaggcatttatattcagtgataattccttcccttagatgcatagggagagtctctaaatttgatggaaatggacacttgagtagtgacttagccttatgtactctgttggaatttgtgctagcagtttgagcactagttctgtgtgcctaggaagttaatgctgcttattgtctcattctgacttcatggagaattaatcccacctttaagcaaaggctactaagttaatggtattttctgtgcagaaattaaattttattttcagcatttagcccaggaattcttccagtaggtgctcagctatttaaaaacaaaactattctcaaacattcatcattagacaactggagtttttgctggttttgtaacctaccaaaatggataggctgttgaacattccacattcaaaagttttgtagggtggtgggaaatgggggatcttcaatgtttattttaaaataaaataaaataagttcttgacttttctcatgtgtggttgtggtacatcatattggaagggttaacctgttactttggcaaatgagtatttttttgctagcacctccccttgcgtgctttaaatgacatctgcctgggatgtaccacaaccatatgttacctgtatcttaggggaatggataaaatatttgtggtttactgggtaatccctagatgatgtatgcttgcagtcctatataaaactaaatttgctatctgtgtagaaaataatttcatgacatttacaatcaggactgaagtaagttcttcacacagtgacctctgaatcagtttcagagaagggatgggggagaaaatgccttctaggttttgaacttctatgcattagtgcagatgttgtgaatgtgtaaaggtgttcatagtttgactgtttctatgtatgttttttcaaagaattgttcctttttttgaactataatttttctttttttggttattttaccatcacagtttaaatgtatatcttttatgtctctactcagaccatatttttaaaggggtgcctcattatggggcagagaacttttcaataagtctcattaagatctgaatcttggttctaagcattctgtataatatgtgattgcttgtcctagctgcagaaggccttttgtttggtcaaatgcatattttagcagagtttcaaggaaatgattgtcacacatgtcactgtagcctcttggtgtagcaagctcacatacaaaatacttttgtatatgcataatataaatcatctcatgtggatatgaaacttcttttttaaaacttaaaaaggtagaatgttattgattaccttgattagggcagttttatttccagatcctaataattcctaaaaaatatggaaaagttttttttcaatcattgtaccttgatattaaaacaaatatcctttaagtatttctaatcagttagcttctacagttcttttgtctccttttatatgcagctcttacgtgggagacttttccacttaaaggagacatagaatgtgtgcttattctcagaaggttcattaactgaggtgatgagttaacaactagttgagcagtcagcttcctaagtgttttaggacatttgttcattatattttccgtcatataactagaggaagtggaatgcagataagtgccgaattcaaacccttcattttatgtttaagctcctgaatctgcattccacttgggttgtttttaagcattctaaattttagttgattataagttagatttcacagaatcagtattgcccttgatcttgtcctttttatggagttaacggggaggaagacccctcaggaaaacgaaagtaaattgttaaggctcatcttcatacctttttccattttgaatcctacaaaaatactgcaaaagactagtgaatgtttaaaattacactagattaaataatatgaaagtc

SEQ ID NO: 2 - protein, 709 aa, Homo sapiens
MPSATSHSGSGSKSSGPPPPSGSSGSEAAAGAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDEVRTDLKQGLNGVPILSEEELSLLDEFYKLVDPERDMSLRLNEQYEHASIHLWDLLEGKEKPVCGTTYKVLKEIVERVFQSNYFDSTHNHQNGLCEEEEAASAPAVEDQVPEAEPEPAEEYTEQSEVESTEYVNRQFMAETQFTSGEKEQVDEWTVETVEVVNSLQQQPQAASPSVPEPHSLTPVAQADPLVRRQRVQDLMAQMQGPYNFIQDSMLDFENQTLDPAIVSAQPMNPTQNMDMPQLVCPPVHSESRLAQPNQVPVQPEATQVPLVSSTSEGYTASQPLYQPSHATEQRPQKEPIDQIQATISLNTDQTTASSSLPAASQPQVFQAGTSKPLHSSGINVNAAPFQSMQTVFNMNAPVPPVNEPETLKQQNQYQASYNQSFSSQPHQVEQTELQQEQLQTVVGTYHGSPDQSHQVTGNHQQPPQQNTGFPRSNQPYYNSRGVSRGGSRGARGLMNGYRGPANGFRGGYDGYRPSFSNTPNSGYTQSQFSAPRDYSGYQRDGYQQNFKRGSGQSGPRGAPRGRGGPPRPNRGMPQMNTQQVN

SEQ ID NO: 3 - DNA, 3553 bp, Homo sapiens
  source 1..3553: mol_type = other DNA
  CDS 190..2274: protein_id = 311; translation = MPSATSHSGSGSKSSGPPPPSGSSGSEAAAGAGAAAPASQHPAT GTGAVQTEAMKQILGVIDKKLRNLEKKKGKLDDYQERMNKGERLNQDQLDAVSKYQEV TNNLEFAKELQRSFMALSQDIQKTIKKTARREQLMREEAEQKRLKTVLELQYVLDKLG DDEVRTDLKQGLNGVPILSEEELSLLDEFYKLVDPERDMSLRLNEQYEHASIHLWDLL EGKEKPVCGTTYKVLKEIVERVFQSNYFDSTHNHQNGLCEEEEAASAPAVEDQVPEAE PEPAEEYTEQSEVESTEYVNRQFMAETQFTSGEKEQVDEWTVETVEVVNSLQQQPQAA SPSVPEPHSLTPVAQADPLVRRQRVQDLMAQMQGPYNFIQDSMLDFENQTLDPAIVSA QPMNPTQNMDMPQLVCPPVHSESRLAQPNQVPVQPEATQVPLVSSTSEGYTASQPLYQ PSHATEQRPQKEPIDQIQATISLNTDQTTASSSLPAASQPQVFQAGTSKPLHSSGINV NAAPFQSMQTVFNMNAPVPPVNEPETLKQQNQYQASYNQSFSSQPHQVEQTELQQEQL QTVVGTYHGSPDQSHQVTGNHQQPPQQNTGFPRSNQPYYNSRGVSRGGSRGARGLMNG YRGPANGFRGGYDGYRPSFSNTPNSGYTQSQFSAPRDYSGYQRDGYQQNFKRGSGQSG PRGAPRGNILWW
cagagggctgctggctggctaagtccctcccgctcccggctctcgcctcactaggagcggctctcggtgcagcgggacagggcgaagcggcctgcgcccacggagcgcgcgacactgcccggaagggaccgccacccttgccccctcagctgcccactcgtgatttccagcggcctccgcgcgcgcacgatgccctcggccaccagccacagcgggagcggcagcaagtcgtccggaccgccaccgccgtcgggttcctccgggagtgaggcggccgcgggagccggggccgccgcgccggcttctcagcaccccgcaaccggcaccggcgctgtccagaccgaggccatgaagcagattctcggggtgatcgacaagaaacttcggaacctggagaagaaaaagggtaagcttgatgattaccaggaacgaatgaacaaaggggaaaggcttaatcaagatcagctggatgccgtttctaagtaccaggaagtcacaaataatttggagtttgcaaaagaattacagaggagtttcatggcactaagtcaagatattcagaaaacaataaagaagacagcacgtcgggagcagcttatgagagaagaagctgaacagaaacgtttaaaaactgtacttgagctacagtatgttttggacaaattgggagatgatgaagtgcggactgacctgaaacaaggtttgaatggagtgccaatattgtccgaagaggagttgtcattgttggatgaattctataagctagtagaccctgaacgggacatgagcttgaggttgaatgaacagtatgaacatgcctccattcacctgtgggacctgctggaagggaaggaaaaacctgtatgtggaaccacctataaagttctaaaggaaattgttgagcgtgtttttcagtcaaactactttgacagcacccacaaccaccagaatgggctgtgtgaggaagaagaggcagcctcagcacctgcagttgaagaccaggtacctgaagctgaacctgagccagcagaagagtacactgagcaaagtgaagttgaatcaacagagtatgtaaatagacagttcatggcagaaacacagttcaccagtggtgaaaaggagcaggtagatgagtggacagttgaaacggttgaggtggtaaattcactccagcagcaacctcaggctgcatccccttcagtaccagagccccactctttgactccagtggctcaggcagatccccttgtgagaagacagcgagtacaagaccttatggcacaaatgcagggtccctataatttcatacaggattcaatgctggattttgaaaatcagacacttgatcctgccattgtatctgcacagcctatgaatccaacacaaaacatggacatgccccagctggtttgccctccagttcattctgaatctagacttgctcagcctaatcaagttcctgtacaaccagaagcgacacaggttcctttggtatcatccacaagtgaggggtacacagcatctcaacccttgtaccagccttctcatgctacagagcaacgaccacagaaggaaccaattgatcagattcaggcaacaatctctttaaatacagaccagactacagcatcatcatcccttcctgctgcgtctcagcctcaagtatttcaggctgggacaagcaaacctttacatagcagtggaatcaatgtaaatgcagctccattccaatccatgcaaacggtgttcaatatgaatgccccagttcctcctgttaatgaaccagaaactttaaaacagcaaaatcagtaccaggccagttataaccagagcttttctagtcagcctcaccaagtagaacaaacagagcttcagcaagaacagcttcaaacagtggttggcacttaccatggttccccagaccagtcccatcaagtgactggtaaccaccagcagcctcctcagcagaacactggatttccacgtagcaatcagccctattacaatagtcgtggtgtgtctcgtggaggctcccgtggtgctagaggcttgatgaatggataccggggccctgccaatggattcagaggaggatatgatggttaccgcccttcattctctaacactccaaacagtggttatacacagtctcagttcagtgctccccgggattactctggctatcaacgggatggatatcagcagaatttcaagcgaggctctgggcagagtggaccacggggagccccacgaggtaatattttgtggtggtgatcctagctcctaagtggagcttctgttctggccttggaagagctgttaatagtctgcatgttaggaatacatttatcctttccagacttgttgctagggattaaatgaaatgctctgtttctaaaacttaatcttggacccaaattttaatttttgaatgatttaattttccctgttactatataaactgtcttgaaaactagaacatattctcttctcagaaaaagtgtttttccaactgaaaattatttttcaggtcctaaaacctgctaaatgtttttaggaagtacttactgaaacatttttgtaagacatttttggaatgagattgaacatttatataaatttattattcctctttcatttttttgaaacatgcctattatattttagggccagacaccctttaatggccggataagccatagttaacatttagagaaccatttagaagtgatagaactaatggaatttgcaatgccttttggacctctattagtgatataaatatcaagttatttctgacttttaaacaaaactcccaaattcctaacttattgagctatacttaaaaaaaattacaggtttagagagttttttgtttttcttttactgttggaaaactacttcccattttggcaggaagttaacctatttaacaattagagctagcatttcatgtagtctgaaattctaaatggttctctgatttgagggaggttaaacatcaaacaggtttcctctattggccataacatgtataaaatgtgtgttaaggaggaattacaacgtactttgatttgaatactagtagaaactggccaggaaaaaggtacatttttctaaaaattaatggatcacttgggaattactgacttgactagaagtatcaaaggatgtttgcatgtgaatgtgggttatgttctttcccaccttgtagcatattcgatgaaagttgagttaactgatagctaaaaatctgttttaacagcatgtaaaaagttattttatctgttaaaagtcattatacagttttgaatgttatgtagtttctttttaacagtttaggtaataaggtctgttttcattctggtgcttttattaattttgatagtatgatgttacttactactgaaatgtaagctagagtgtacactagaatgtaagctccatgagagcaggtaccttgtctgtcttctctgctgtatctattcccaacgcttgatgatggtgcctggcacatagtaggcactcaataaatatttgttgaatgaatgaa

SEQ ID NO: 4 - protein, 694 aa, Homo sapiens
MPSATSHSGSGSKSSGPPPPSGSSGSEAAAGAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDEVRTDLKQGLNGVPILSEEELSLLDEFYKLVDPERDMSLRLNEQYEHASIHLWDLLEGKEKPVCGTTYKVLKEIVERVFQSNYFDSTHNHQNGLCEEEEAASAPAVEDQVPEAEPEPAEEYTEQSEVESTEYVNRQFMAETQFTSGEKEQVDEWTVETVEVVNSLQQQPQAASPSVPEPHSLTPVAQADPLVRRQRVQDLMAQMQGPYNFIQDSMLDFENQTLDPAIVSAQPMNPTQNMDMPQLVCPPVHSESRLAQPNQVPVQPEATQVPLVSSTSEGYTASQPLYQPSHATEQRPQKEPIDQIQATISLNTDQTTASSSLPAASQPQVFQAGTSKPLHSSGINVNAAPFQSMQTVFNMNAPVPPVNEPETLKQQNQYQASYNQSFSSQPHQVEQTELQQEQLQTVVGTYHGSPDQSHQVTGNHQQPPQQNTGFPRSNQPYYNSRGVSRGGSRGARGLMNGYRGPANGFRGGYDGYRPSFSNTPNSGYTQSQFSAPRDYSGYQRDGYQQNFKRGSGQSGPRGAPRGNILWW

SEQ ID NO: 5 - DNA, 1605 bp, Canis lupus
  source 1..1605: mol_type = other DNA; note = Canis familiaris
  CDS 46..1392: protein_id = 312; translation = MALSQDIQKTIKKTARREQLMREEAEQKRLKTVLELQYVLDKLG DDEVRTDLKQGLNGVPILSEEELSLLDEFYKLADPERDMSLRLNEQYEHASIHLWDLL EGKEKSVCGTTYKALKEIVERVFQSNYFDSTHNHQNGLCEEEEAASAPTVEDQVAEAE PEPAEEYTEQSEVESTEYVNRQFMAETQFSSGEKEQVDEWTVETVEVVNSLQQQPQAA SPSVPEPHSLTPVAQADPLVRRQRVQDLMAQMQGPYNFIQDSMLDFENQTLDPAIVSA QPMNPTQNMDMPQLVCPPVHSESRLAQPNQVPVQPEATQVPLVSSTSEGYTASQPLYQ PSHATEQRPQKEPIDQIQATISLNTDQTTASSSLPAASQPQVFQAGTSKPLHSSGINV NAAPFQSMQTVFNMNAPVPPVNEPETLKQQNQYQASYNQSFSSQPHQVEQTEGCRK
gtcacaaataacttggagtttgcaaaagaattacagaggagtttcatggcattaagtcaagatattcagaaaacaataaagaagactgcacgtcgggagcagcttatgagagaggaagcggaacaaaaacgtttaaaaactgtacttgagctccagtatgttttggacaaattgggagatgatgaagtgagaactgacctgaagcaaggtttgaatggagtgccaatattgtctgaagaagaattgtcgttgttggatgaattctacaaattagcagaccctgaacgggacatgagcttgaggttgaatgagcagtatgaacatgcttccattcacctgtgggacttgctggaaggaaaggaaaagtctgtatgtggaacaacctataaagcactaaaggaaattgttgagcgtgttttccagtcaaattactttgacagcactcacaaccaccagaatgggctatgtgaggaagaagaggcagcctcagcacctacagttgaagaccaggtagctgaagctgagcctgagccagcagaagaatacactgaacaaagtgaagttgaatcaacagagtatgtaaatagacaatttatggcagaaacacagttcagcagtggtgaaaaggagcaggtagatgagtggacggtcgaaacagtggaggtggtgaattcactccagcagcaacctcaggctgcgtctccttcagtaccagagccccactctttgactccggtggctcaggcagatccccttgtgagaagacagcgagtccaggaccttatggcgcagatgcaggggccctataatttcatacaggattcaatgctggattttgaaaaccagacactcgatcctgccattgtatctgcacagcctatgaatccgacacaaaacatggacatgccccagctggtttgccctccagttcattctgaatctagacttgctcaacctaatcaagttcctgtacaaccagaagctacacaggttcctttggtttcatccacaagtgaggggtatacagcatctcaacccttgtaccagccttctcatgctacagagcaacgaccacaaaaggaaccaattgaccagattcaggcaacaatctctttaaatacagaccagactacagcgtcatcatcccttccggctgcttctcagcctcaggtattccaggctgggacaagcaaaccattacatagcagtggaatcaatgtaaatgcagctccattccaatccatgcaaacggtgttcaatatgaatgccccagttcctcctgttaatgaaccagaaactttgaaacaacaaaatcagtaccaggccagttataaccagagcttttctagtcagcctcaccaagtagaacaaacagagggatgccgcaaatgaacactcagcaagtgaattaatctgattcacaggattatgtttaaacgccaaaaacacactggccagtgtaccataatatgttaccagaagagttattatctatttgttctccctttcaggaaacttattgtaaagggactgttttcatcccataaagacaggactacaattgtcagctttatattacctggaaaaaaaaaaaaaaaaaaaaaa

SEQ ID NO: 6 - protein, 448 aa, Canis lupus
  source 1..448: note = Canis familiaris
MALSQDIQKTIKKTARREQLMREEAEQKRLKTVLELQYVLDKLGDDEVRTDLKQGLNGVPILSEEELSLLDEFYKLADPERDMSLRLNEQYEHASIHLWDLLEGKEKSVCGTTYKALKEIVERVFQSNYFDSTHNHQNGLCEEEEAASAPTVEDQVAEAEPEPAEEYTEQSEVESTEYVNRQFMAETQFSSGEKEQVDEWTVETVEVVNSLQQQPQAASPSVPEPHSLTPVAQADPLVRRQRVQDLMAQMQGPYNFIQDSMLDFENQTLDPAIVSAQPMNPTQNMDMPQLVCPPVHSESRLAQPNQVPVQPEATQVPLVSSTSEGYTASQPLYQPSHATEQRPQKEPIDQIQATISLNTDQTTASSSLPAASQPQVFQAGTSKPLHSSGINVNAAPFQSMQTVFNMNAPVPPVNEPETLKQQNQYQASYNQSFSSQPHQVEQTEGCRK

SEQ ID NO: 7 - DNA, 4154 bp, Canis lupus
  source 1..4154: mol_type = other DNA; note = Canis familiaris
  CDS 1..2154: protein_id = 313; translation = MPSATSLSGSGSKSSGPPPPSGSSGSEAAAAAGAAGAAGAGAAA PASQHPATGTGAVQTEAMKQILGVIDKKLRNLEKKKGKLDDYQERMNKGERLNQDQLD AVSKYQEVTNNLEFAKELQRSFMALSQDIQKTIKKTARREQLMREEAEQKRLKTVLEL QYVLDKLGDDEVRTDLKQGLNGVPILSEEELSLLDEFYKLADPERDMSLRLNEQYEHA SIHLWDLLEGKEKSVCGTTYKALKEIVERVFQSNYFDSTHNHQNGLCEEEEAASAPTV EDQVAEAEPEPAEEYTEQSEVESTEYVNRQFMAETQFSSGEKEQVDEWTVETVEVVNS LQQQPQAASPSVPEPHSLTPVAQADPLVRRQRVQDLMAQMQGPYNFIQDSMLDFENQT LDPAIVSAQPMNPTQNMDMPQLVCPPVHSESRLAQPNQVPVQPEATQVPLVSSTSEGY TASQPLYQPSHATEQRPQKEPIDQIQATISLNTDQTTASSSLPAASQPQVFQAGTSKP LHSSGINVNAAPFQSMQTVFNMNAPVPPVNEPETLKQQNQYQASYNQSFSSQPHQVEQ TDLQQEQLQTVVGTYHGSQDQPHQVTGNHQQPPQQNTGFPRSSQPYYNSRGVSRGGSR GARGLMNGYRGPANGFRGGYDGYRPSFSNTPNSGYTQSQFSAPRDYSGYQRDGYQQNF KRGSGQSGPRGAPRGRGGPPRPNRGMPQMNTQQVN
atgccgtcggccaccagcctcagcggaagcggcagcaagtcgtcgggcccgccgcccccgtcgggttcctccgggagcgaggcggcggcggcggcgggggcggcgggggcggcgggggccggggcggctgcgcccgcctcccagcaccccgcgaccggcaccggcgctgtccagaccgaggccatgaagcagatcctcggggtgatcgacaagaaactccggaacctggagaagaaaaagggcaagcttgatgattaccaggaacgaatgaacaaaggggaaaggcttaatcaagatcagctggatgccgtatctaagtaccaggaagtcacaaataacttggagtttgcaaaagaattacagaggagtttcatggcattaagtcaagatattcagaaaacaataaagaagactgcacgtcgggagcagcttatgagagaggaagcggaacaaaaacgtttaaaaactgtacttgagctccagtatgttttggacaaattgggagatgatgaagtgagaactgacctgaagcaaggtttgaatggagtgccaatattgtctgaagaagaattgtcgttgttggatgaattctacaaattagcagaccctgaacgggacatgagcttgaggttgaatgagcagtatgaacatgcttccattcacctgtgggacttgctggaaggaaaggaaaagtctgtatgtggaacaacctataaagcactaaaggaaattgttgagcgtgttttccagtcaaattactttgacagcactcacaaccaccagaatgggctatgtgaggaagaagaggcagcctcagcacctacagttgaagaccaggtagctgaagctgagcctgagccagcagaagaatacactgaacaaagtgaagttgaatcaacagagtatgtaaatagacaatttatggcagaaacacagttcagcagtggtgaaaaggagcaggtagatgagtggacggtcgaaacagtggaggtggtgaattcactccagcagcaacctcaggctgcgtctccttcagtaccagagccccactctttgactccggtggctcaggcagatccccttgtgagaagacagcgagtccaggaccttatggcgcagatgcaggggccctataatttcatacaggattcaatgctggattttgaaaaccagacactcgatcctgccattgtatctgcacagcctatgaatccgacacaaaacatggacatgccccagctggtttgccctccagttcattctgaatctagacttgctcaacctaatcaagttcctgtacaaccagaagctacacaggttcctttggtttcatccacaagtgaggggtatacagcatctcaacccttgtaccagccttctcatgctacagagcaacgaccacaaaaggaaccaattgaccagattcaggcaacaatctctttaaatacagaccagactacagcgtcatcatcccttccggctgcttctcagcctcaggtattccaggctgggacaagcaaaccattacatagcagtggaatcaatgtaaatgcagctccattccaatccatgcaaacggtgttcaatatgaatgccccagttcctcctgttaatgaaccagaaactttgaaacaacaaaatcagtaccaggccagttataaccagagcttttctagtcagcctcaccaagtagaacaaacagaccttcagcaagaacagcttcaaacagtggttggcacttaccatggttcccaggaccagccccaccaagtgactggtaaccatcagcagcctccccagcagaacactggatttccacgtagcagtcagccctattacaatagtcgtggtgtgtctcgtggtggttcccgtggtgctagaggcttaatgaatggatacaggggccctgccaatggattcagaggaggatatgatggttaccgcccttcattctctaacactccaaacagtggttatacacagtctcagttcagtgctccccgggactactctggctatcagcgggatggatatcagcagaatttcaagcgaggctctgggcagagtggaccacggggagccccacgaggtcgtggagggcccccaagacccaacagagggatgccgcaaatgaacactcagcaagtgaattaatctgattcacaggattatgtttaaacgccaaaaacacactggccagtgtaccataatatgttaccagaagagttattatctatttgttctccctttcaggaaacttattgtaaagggactgttttcatcccataaagacaggactacaattgtcagctttatattacctggatatggaaggaaactatttttattctgcatgttcttcctaagcgtcatcttgagccttgcacatgatactcagattcctcacccttgcttaggagtaaaacataatacactttacagggtgatatctccatagttatttgaagtggcttggaaaaagcaagattaacttctgacattggataaaaatcaacaaatcagccctagagttattcaaatggtaattgacaaaaactaaaatatttcccttcgagaaggagtggaatgtggtttggcagaacaactgcatttcacagcttttccggttaaattggagcactaaacgtttagatgcataccaaattatgcatgggcccttaatataaaaggctggctaccagctttgacacagcactattcatcctctggccaaacaactgtggttaaacaacacatgtaaattgctttttaacagctgatactataataagacaaagccaaaatgcaaaaattgggctttgattggcactttttgaaaaatatgcaacaaatatgggatgtaatctggatggccgcttctgtacttaatgtgaagtatttagatacctttttgaacacttaacagtttcttctgacaatgacttttgtaaggattggtactatctatcattccttataatgtacattgtctgtcactaatcctcagatcttgctgtattgtcacctaaattggtacaggtactgatgaaaatatctaatggataatcataacactcttggtcacatgtttttcctgcagcctgaaggtttttaaaagaaaaagatatcaaatgcctgctgctaccacccttttaaattgctatcttttgaaaagcaccagtatgtgttttagattgatttccctattttagggaaatgacagacagtagtttcagttctgatggtataagcaaaacaaataaaacatgtttataaaagttgtatcttgaaacactggtgttcaacagctagcagcttatgtggttcaccccatgcattgttagtgtttcagattttatggttatctccagcagctgtttctgtagtacttgcatttatcttttgtctaaccctaatattctcacggaggcatttatattcaaagtggtgatcccttcacttagacgcatagggagagtcacaagtttgatgaagaggacagtgtagtaatttatatgctgttggaatttgtgctagcagtttgagcactagttctgtgtgcctatgaacttaatgctgcttgtcatattccactttgacttcatggagaattaatcccatctactcagcaaaggctatactaatactaagttaatggtattttctgtgcagaaattgaattttgttttattagcatttagctaaggaatttttccagtaggtgctcagctactaaagaaaaacaaaaacaagacacaaaactattctcaaacattcattgttagacaactggagtttttgctggttttgtaacctactaaaatggataggctgttgaacattccacattcaaaagttttttgtagggtggtggggaagggggggtgtcttcaatgtttattttaaaataaaataagttcttgacttttctcatgtgtggttgtggtacatcatattggaagggttatctgtttacttttgcaaatgagtatttctcttgctagcacctcccgttgtgcgctttaaatgacatctgcctgggatgtaccacaaccatatgttagctgtattttatggggaatagataaaatattcgtggtttattgggtaatccctagatgtgtatgcttacaatcctatatataaaactaaat

SEQ ID NO: 8 - protein, 717 aa, Canis lupus
  source 1..717: note = Canis familiaris
MPSATSLSGSGSKSSGPPPPSGSSGSEAAAAAGAAGAAGAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDEVRTDLKQGLNGVPILSEEELSLLDEFYKLADPERDMSLRLNEQYEHASIHLWDLLEGKEKSVCGTTYKALKEIVERVFQSNYFDSTHNHQNGLCEEEEAASAPTVEDQVAEAEPEPAEEYTEQSEVESTEYVNRQFMAETQFSSGEKEQVDEWTVETVEVVNSLQQQPQAASPSVPEPHSLTPVAQADPLVRRQRVQDLMAQMQGPYNFIQDSMLDFENQTLDPAIVSAQPMNPTQNMDMPQLVCPPVHSESRLAQPNQVPVQPEATQVPLVSSTSEGYTASQPLYQPSHATEQRPQKEPIDQIQATISLNTDQTTASSSLPAASQPQVFQAGTSKPLHSSGINVNAAPFQSMQTVFNMNAPVPPVNEPETLKQQNQYQASYNQSFSSQPHQVEQTDLQQEQLQTVVGTYHGSQDQPHQVTGNHQQPPQQNTGFPRSSQPYYNSRGVSRGGSRGARGLMNGYRGPANGFRGGYDGYRPSFSNTPNSGYTQSQFSAPRDYSGYQRDGYQQNFKRGSGQSGPRGAPRGRGGPPRPNRGMPQMNTQQVN

SEQ ID NO: 9 - DNA, 4939 bp, Canis lupus
  source 1..4939: mol_type = other DNA; note = Canis familiaris
  CDS 1..2109: protein_id = 314; translation = MPSATSLSGSGSKSSGPPPPSGSSGSEAAAAAGAAGAAGAGAAA PASQHPATGTGAVQTEAMKQILGVIDKKLRNLEKKKGKLDDYQERMNKGERLNQDQLD AVSKYQEVTNNLEFAKELQRSFMALSQDIQKTIKKTARREQLMREEAEQKRLKTVLEL QYVLDKLGDDEVRTDLKQGLNGVPILSEEELSLLDEFYKLADPERDMSLRLNEQYEHA SIHLWDLLEGKEKSVCGTTYKALKEIVERVFQSNYFDSTHNHQNGLCEEEEAASAPTV EDQVAEAEPEPAEEYTEQSEVESTEYVNRQFMAETQFSSGEKEQVDEWTVETVEVVNS LQQQPQAASPSVPEPHSLTPVAQADPLVRRQRVQDLMAQMQGPYNFIQDSMLDFENQT LDPAIVSAQPMNPTQNMDMPQLVCPPVHSESRLAQPNQVPVQPEATQVPLVSSTSEGY TASQPLYQPSHATEQRPQKEPIDQIQATISLNTDQTTASSSLPAASQPQVFQAGTSKP LHSSGINVNAAPFQSMQTVFNMNAPVPPVNEPETLKQQNQYQASYNQSFSSQPHQVEQ TDLQQEQLQTVVGTYHGSQDQPHQVTGNHQQPPQQNTGFPRSSQPYYNSRGVSRGGSR GARGLMNGYRGPANGFRGGYDGYRPSFSNTPNSGYTQSQFSAPRDYSGYQRDGYQQNF KRGSGQSGPRGAPRGNILWW
atgccgtcggccaccagcctcagcggaagcggcagcaagtcgtcgggcccgccgcccccgtcgggttcctccgggagcgaggcggcggcggcggcgggggcggcgggggcggcgggggccggggcggctgcgcccgcctcccagcaccccgcgaccggcaccggcgctgtccagaccgaggccatgaagcagatcctcggggtgatcgacaagaaactccggaacctggagaagaaaaagggcaagcttgatgattaccaggaacgaatgaacaaaggggaaaggcttaatcaagatcagctggatgccgtatctaagtaccaggaagtcacaaataacttggagtttgcaaaagaattacagaggagtttcatggcattaagtcaagatattcagaaaacaataaagaagactgcacgtcgggagcagcttatgagagaggaagcggaacaaaaacgtttaaaaactgtacttgagctccagtatgttttggacaaattgggagatgatgaagtgagaactgacctgaagcaaggtttgaatggagtgccaatattgtctgaagaagaattgtcgttgttggatgaattctacaaattagcagaccctgaacgggacatgagcttgaggttgaatgagcagtatgaacatgcttccattcacctgtgggacttgctggaaggaaaggaaaagtctgtatgtggaacaacctataaagcactaaaggaaattgttgagcgtgttttccagtcaaattactttgacagcactcacaaccaccagaatgggctatgtgaggaagaagaggcagcctcagcacctacagttgaagaccaggtagctgaagctgagcctgagccagcagaagaatacactgaacaaagtgaagttgaatcaacagagtatgtaaatagacaatttatggcagaaacacagttcagcagtggtgaaaaggagcaggtagatgagtggacggtcgaaacagtggaggtggtgaattcactccagcagcaacctcaggctgcgtctccttcagtaccagagccccactctttgactccggtggctcaggcagatccccttgtgagaagacagcgagtccaggaccttatggcgcagatgcaggggccctataatttcatacaggattcaatgctggattttgaaaaccagacactcgatcctgccattgtatctgcacagcctatgaatccgacacaaaacatggacatgccccagctggtttgccctccagttcattctgaatctagacttgctcaacctaatcaagttcctgtacaaccagaagctacacaggttcctttggtttcatccacaagtgaggggtatacagcatctcaacccttgtaccagccttctcatgctacagagcaacgaccacaaaaggaaccaattgaccagattcaggcaacaatctctttaaatacagaccagactacagcgtcatcatcccttccggctgcttctcagcctcaggtattccaggctgggacaagcaaaccattacatagcagtggaatcaatgtaaatgcagctccattccaatccatgcaaacggtgttcaatatgaatgccccagttcctcctgttaatgaaccagaaactttgaaacaacaaaatcagtaccaggccagttataaccagagcttttctagtcagcctcaccaagtagaacaaacagaccttcagcaagaacagcttcaaacagtggttggcacttaccatggttcccaggaccagccccaccaagtgactggtaaccatcagcagcctccccagcagaacactggatttccacgtagcagtcagccctattacaatagtcgtggtgtgtctcgtggtggttcccgtggtgctagaggcttaatgaatggatacaggggccctgccaatggattcagaggaggatatgatggttaccgcccttcattctctaacactccaaacagtggttatacacagtctcagttcagtgctccccgggactactctggctatcagcgggatggatatcagcagaatttcaagcgaggctctgggcagagtggaccacggggagccccacgaggtaatattttgtggtggtgatcctagctcctaagtggagcttctgttctggccttggaagagctgttccatagtctgcatgtaggttacatgttaggaatacatttatcattaccagacttgttgctagggattaaatgaaatgctctgtttctaaaacttctcttgaacccaaatttaattttttgaatgactttccctgttactatataaattgtcttgaaaactagaacatttctcctcctcagaaaaagtgtttttccaactgcaaattatttttcaggtcctaaaacctgctaaatgtttttaggaagtacttactgaaacatttttgtaagacatttttggaatgagattgaacatttatataaatttattattattcctctttcatttttgaacatgcatattatattttagggtcagaaatcctttaatggccaaataagccatagttacatttagagaaccatttagaagtgatagaactaactgaaatttcaatgcctttggatcattaatagcgatataaatttcaaattgtttctgacttttaaataaaacatccaaaatcctaactaacttcctgaactatatttaaaaattacaggtttaaggagtttctggttttttttctcttaccataggaaaactgtttcctgtttggccaggaagtcaacctgtgtaataattagaagtagcatttcatatgatctgaagttctaaatggttctctgatttaagggaagttaaattgaataggtttcctctagttattggccataacatgtataaaatgtatattaaggaggaatacaaagtactttgatttcaatgctagtagaaactggccagcaaaaaggtgcattttatttttaaattaatggatcacttgggaattactgacttgaagtatcaaaggatatttgcatgtgaatgtgggttatgttctttctcaccttgtagcatattctatgaaagttgagttgactggtagctaaaaatctgttttaacagcatgtaaaaagttattttatctgttacaagtcattatacaattttgaatgttatgtagtttctttttaacagtttaggtaacaaggtctgtttttcattctggtgcttttattaattttgatagtatgatgttacttactactgaaatgtaagctagagtgtacactagaatgtaagctccatgagagcaggtaccttgtctgtcttcactgctgtatctatttccaacgcctgatgacagtgcctgacacatagtaggcactcaataaatacttgttgaatgaatgaatgaatgagtactggtggaatactccattagctctactcttcttttagctagagaacatgagcaaatttgcgcatgacaacttccaggacaggtgaacactgaagaattgacctcttaaacctaataatgtggtgacaagctgcccacatgcttcttgacttcagatgaaaatctgcttgaaggcaaagcaaataatatttgaaagaaaaaccaaatgccatttttgtcttctaggtcgtggagggcccccaagacccaacagagggatgccgcaaatgaacactcagcaagtgaattaatctgattcacaggattatgtttaaacgccaaaaacacactggccagtgtaccataatatgttaccagaagagttattatctatttgttctccctttcaggaaacttattgtaaagggactgttttcatcccataaagacaggactacaattgtcagctttatattacctggatatggaaggaaactatttttattctgcatgttcttcctaagcgtcatcttgagccttgcacatgatactcagattcctcacccttgcttaggagtaaaacataatacactttacagggtgatatctccatagttatttgaagtggcttggaaaaagcaagattaacttctgacattggataaaaatcaacaaatcagccctagagttattcaaatggtaattgacaaaaactaaaatatttcccttcgagaaggagtggaatgtggtttggcagaacaactgcatttcacagcttttccggttaaattggagcactaaacgtttagatgcataccaaattatgcatgggcccttaatataaaaggctggctaccagctttgacacagcactattcatcctctggccaaacaactgtggttaaacaacacatgtaaattgctttttaacagctgatactataataagacaaagccaaaatgcaaaaattgggctttgattggcactttttgaaaaatatgcaacaaatatgggatgtaatctggatggccgcttctgtacttaatgtgaagtatttagatacctttttgaacacttaacagtttcttctgacaatgacttttgtaaggattggtactatctatcattccttataatgtacattgtctgtcactaatcctcagatcttgctgtattgtcacctaaattggtacaggtactgatgaaaatatctaatggataatcataacactcttggtcacatgtttttcctgcagcctgaaggtttttaaaagaaaaagatatcaaatgcctgctgctaccacccttttaaattgctatcttttgaaaagcaccagtatgtgttttagattgatttccctattttagggaaatgacagacagtagtttcagttctgatggtataagcaaaacaaataaaacatgtttataaaagttgtatcttgaaacactggtgttcaacagctagcagcttatgtggttcaccccatgcattgttagtgtttcagattttatggttatctccagcagctgtttctgtagtacttgcatttatc

SEQ ID NO: 10 - protein, 702 aa, Canis lupus
  source 1..702: note = Canis familiaris
MPSATSLSGSGSKSSGPPPPSGSSGSEAAAAAGAAGAAGAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDEVRTDLKQGLNGVPILSEEELSLLDEFYKLADPERDMSLRLNEQYEHASIHLWDLLEGKEKSVCGTTYKALKEIVERVFQSNYFDSTHNHQNGLCEEEEAASAPTVEDQVAEAEPEPAEEYTEQSEVESTEYVNRQFMAETQFSSGEKEQVDEWTVETVEVVNSLQQQPQAASPSVPEPHSLTPVAQADPLVRRQRVQDLMAQMQGPYNFIQDSMLDFENQTLDPAIVSAQPMNPTQNMDMPQLVCPPVHSESRLAQPNQVPVQPEATQVPLVSSTSEGYTASQPLYQPSHATEQRPQKEPIDQIQATISLNTDQTTASSSLPAASQPQVFQAGTSKPLHSSGINVNAAPFQSMQTVFNMNAPVPPVNEPETLKQQNQYQASYNQSFSSQPHQVEQTDLQQEQLQTVVGTYHGSQDQPHQVTGNHQQPPQQNTGFPRSSQPYYNSRGVSRGGSRGARGLMNGYRGPANGFRGGYDGYRPSFSNTPNSGYTQSQFSAPRDYSGYQRDGYQQNFKRGSGQSGPRGAPRGNILWW

SEQ ID NO: 11 - DNA, 3306 bp, Canis lupus
  source 1..3306: mol_type = other DNA; note = Canis familiaris
  CDS 1..2040: protein_id = 315; translation = MPSATSLSGSGSKSSGPPPPSGSSGSEAAAAAGAAGAAGAGAAA PASQHPATGTGAVQTEAMKQILGVIDKKLRNLEKKKGKLDDYQERMNKGERLNQDQLD AVSKYQEVTNNLEFAKELQRSFMALSQDIQKTIKKTARREQLMREEAEQKRLKTVLEL QYVLDKLGDDEVRTDLKQGLNGVPILSEEELSLLDEFYKLADPERDMSLRLNEQYEHA SIHLWDLLEGKEKSVCGTTYKALKEIVERVFQSNYFDSTHNHQNGLCEEEEAASAPTV EDQVAEAEPEPAEEYTEQSEVESTEYVNRQFMAETQFSSGEKEQVDEWTVETVEVVNS LQQQPQAASPSVPEPHSLTPVAQADPLVRRQRVQDLMAQMQGPYNFIQDSMLDFENQT LDPAIVSAQPMNPTQNMDMPQLVCPPVHSESRLAQPNQVPVQPEATQVPLVSSTSEGY TASQPLYQPSHATEQRPQKEPIDQIQATISLNTDQTTASSSLPAASQPQVFQAGTSKP LHSSGINVNAAPFQSMQTVFNMNAPVPPVNEPETLKQQNQYQASYNQSFSSQPHQVEQ TDLQQEQLQTVVGTYHGSQDQPHQVTGNHQQPPQQNTGFPRSSQPYYNSRGVSRGGSR GARGLMNGYRGPANGFRGGYDGYRPSFSNTPNSGYTQSQFSAPRDYSGYQRGCRK
atgccgtcggccaccagcctcagcggaagcggcagcaagtcgtcgggcccgccgcccccgtcgggttcctccgggagcgaggcggcggcggcggcgggggcggcgggggcggcgggggccggggcggctgcgcccgcctcccagcaccccgcgaccggcaccggcgctgtccagaccgaggccatgaagcagatcctcggggtgatcgacaagaaactccggaacctggagaagaaaaagggcaagcttgatgattaccaggaacgaatgaacaaaggggaaaggcttaatcaagatcagctggatgccgtatctaagtaccaggaagtcacaaataacttggagtttgcaaaagaattacagaggagtttcatggcattaagtcaagatattcagaaaacaataaagaagactgcacgtcgggagcagcttatgagagaggaagcggaacaaaaacgtttaaaaactgtacttgagctccagtatgttttggacaaattgggagatgatgaagtgagaactgacctgaagcaaggtttgaatggagtgccaatattgtctgaagaagaattgtcgttgttggatgaattctacaaattagcagaccctgaacgggacatgagcttgaggttgaatgagcagtatgaacatgcttccattcacctgtgggacttgctggaaggaaaggaaaagtctgtatgtggaacaacctataaagcactaaaggaaattgttgagcgtgttttccagtcaaattactttgacagcactcacaaccaccagaatgggctatgtgaggaagaagaggcagcctcagcacctacagttgaagaccaggtagctgaagctgagcctgagccagcagaagaatacactgaacaaagtgaagttgaatcaacagagtatgtaaatagacaatttatggcagaaacacagttcagcagtggtgaaaaggagcaggtagatgagtggacggtcgaaacagtggaggtggtgaattcactccagcagcaacctcaggctgcgtctccttcagtaccagagccccactctttgactccggtggctcaggcagatccccttgtgagaagacagcgagtccaggaccttatggcgcagatgcaggggccctataatttcatacaggattcaatgctggattttgaaaaccagacactcgatcctgccattgtatctgcacagcctatgaatccgacacaaaacatggacatgccccagctggtttgccctccagttcattctgaatctagacttgctcaacctaatcaagttcctgtacaaccagaagctacacaggttcctttggtttcatccacaagtgaggggtatacagcatctcaacccttgtaccagccttctcatgctacagagcaacgaccacaaaaggaaccaattgaccagattcaggcaacaatctctttaaatacagaccagactacagcgtcatcatcccttccggctgcttctcagcctcaggtattccaggctgggacaagcaaaccattacatagcagtggaatcaatgtaaatgcagctccattccaatccatgcaaacggtgttcaatatgaatgccccagttcctcctgttaatgaaccagaaactttgaaacaacaaaatcagtaccaggccagttataaccagagcttttctagtcagcctcaccaagtagaacaaacagaccttcagcaagaacagcttcaaacagtggttggcacttaccatggttcccaggaccagccccaccaagtgactggtaaccatcagcagcctccccagcagaacactggatttccacgtagcagtcagccctattacaatagtcgtggtgtgtctcgtggtggttcccgtggtgctagaggcttaatgaatggatacaggggccctgccaatggattcagaggaggatatgatggttaccgcccttcattctctaacactccaaacagtggttatacacagtctcagttcagtgctccccgggactactctggctatcagcggggatgccgcaaatgaacactcagcaagtgaattaatctgattcacaggattatgtttaaacgccaaaaacacactggccagtgtaccataatatgttaccagaagagttattatctatttgttctccctttcaggaaacttattgtaaagggactgttttcatcccataaagacaggactacaattgtcagctttatattacctggatatggaaggaaactatttttattctgcatgttcttcctaagcgtcatcttgagccttgcacatgatactcagattcctcacccttgcttaggagtaaaacataatacactttacagggtgatatctccatagttatttgaagtggcttggaaaaagcaagattaacttctgacattggataaaaatcaacaaatcagccctagagttattcaaatggtaattgacaaaaactaaaatatttcccttcgagaaggagtggaatgtggtttggcagaacaactgcatttcacagcttttccggttaaattggagcactaaacgtttagatgcataccaaattatgcatgggcccttaatataaaaggctggctaccagctttgacacagcactattcatcctctggccaaacaactgtggttaaacaacacatgtaaattgctttttaacagctgatactataataagacaaagccaaaatgcaaaaattgggctttgattggcactttttgaaaaatatgcaacaaatatgggatgtaatctggatggccgcttctgtacttaatgtgaagtatttagatacctttttgaacacttaacagtttcttctgacaatgacttttgtaaggattggtactatctatcattccttataatgtacattgtctgtcactaatcctcagatcttgctgtattgtcacctaaattggtacaggtactgatgaaaatatctaatggataatcataacactcttggtcacatgtttttcctgcagcctgaaggtttttaaaagaaaaagatatcaaatgcctgctgctaccacccttttaaattgctatcttttgaaaagcaccagtatgtgttttagattgatttccctattttagggaaatgacagacagtagtttcagttctgatggtataagcaaaacaaataaaacatgtttataaaagttgtatcttgaaacactggtgttcaacagctagcagcttatgtggttcaccccatgcattgttagtgtttcagattttatggttatctccagcagctgtttctgtagtacttgcatttatc

SEQ ID NO: 12 - protein, 679 aa, Canis lupus
  source 1..679: note = Canis familiaris
MPSATSLSGSGSKSSGPPPPSGSSGSEAAAAAGAAGAAGAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDEVRTDLKQGLNGVPILSEEELSLLDEFYKLADPERDMSLRLNEQYEHASIHLWDLLEGKEKSVCGTTYKALKEIVERVFQSNYFDSTHNHQNGLCEEEEAASAPTVEDQVAEAEPEPAEEYTEQSEVESTEYVNRQFMAETQFSSGEKEQVDEWTVETVEVVNSLQQQPQAASPSVPEPHSLTPVAQADPLVRRQRVQDLMAQMQGPYNFIQDSMLDFENQTLDPAIVSAQPMNPTQNMDMPQLVCPPVHSESRLAQPNQVPVQPEATQVPLVSSTSEGYTASQPLYQPSHATEQRPQKEPIDQIQATISLNTDQTTASSSLPAASQPQVFQAGTSKPLHSSGINVNAAPFQSMQTVFNMNAPVPPVNEPETLKQQNQYQASYNQSFSSQPHQVEQTDLQQEQLQTVVGTYHGSQDQPHQVTGNHQQPPQQNTGFPRSSQPYYNSRGVSRGGSRGARGLMNGYRGPANGFRGGYDGYRPSFSNTPNSGYTQSQFSAPRDYSGYQRGCRK

SEQ ID NO: 13 - DNA, 2281 bp, Canis lupus
  source 1..2281: mol_type = other DNA; note = Canis familiaris
  CDS 1..2154: protein_id = 316; translation = MPSATSLSGSGSKSSGPPPPSGSSGSEAAAAAGAAGAAGAGAAA PASQHPATGTGAVQTEAMKQILGVIDKKLRNLEKKKGKLDDYQERMNKGERLNQDQLD AVSKYQEVTNNLEFAKELQRSFMALSQDIQKTIKKTARREQLMREEAEQKRLKTVLEL QYVLDKLGDDEVRTDLKQGLNGVPILSEEELSLLDEFYKLADPERDMSLRLNEQYEHA SIHLWDLLEGKEKSVCGTTYKALKEIVERVFQSNYFDSTHNHQNGLCEEEEAASAPTV EDQVAEAEPEPAEEYTEQSEVESTEYVNRQFMAETQFSSGEKEQVDEWTVETVEVVNS LQQQPQAASPSVPEPHSLTPVAQADPLVRRQRVQDLMAQMQGPYNFIQDSMLDFENQT LDPAIVSAQPMNPTQNMDMPQLVCPPVHSESRLAQPNQVPVQPEATQVPLVSSTSEGY TASQPLYQPSHATEQRPQKEPIDQIQATISLNTDQTTASSSLPAASQPQVFQAGTSKP LHSSGINVNAAPFQSMQTVFNMNAPVPPVNEPETLKQQNQYQASYNQSFSSQPHQVEQ TDLQQEQLQTVVGTYHGSQDQPHQVTGNHQQPPQQNTGFPRSSQPYYNSRGVSRGGSR GARGLMNGYRGPANGFRGGYDGYRPSFSNTPNSGYTQSQFSAPRDYSGYQRDGYQQNF KRGSGQSGPRGAPRGRGGPPRPNRGMPQMNTQQVN
atgccgtcggccaccagcctcagcggaagcggcagcaagtcgtcgggcccgccgcccccgtcgggttcctccgggagcgaggcggcggcggcggcgggggcggcgggggcggcgggggccggggcggctgcgcccgcctcccagcaccccgcgaccggcaccggcgctgtccagaccgaggccatgaagcagatcctcggggtgatcgacaagaaactccggaacctggagaagaaaaagggcaagcttgatgattaccaggaacgaatgaacaaaggggaaaggcttaatcaagatcagctggatgccgtatctaagtaccaggaagtcacaaataacttggagtttgcaaaagaattacagaggagtttcatggcattaagtcaagatattcagaaaacaataaagaagactgcacgtcgggagcagcttatgagagaggaagcggaacaaaaacgtttaaaaactgtacttgagctccagtatgttttggacaaattgggagatgatgaagtgagaactgacctgaagcaaggtttgaatggagtgccaatattgtctgaagaagaattgtcgttgttggatgaattctacaaattagcagaccctgaacgggacatgagcttgaggttgaatgagcagtatgaacatgcttccattcacctgtgggacttgctggaaggaaaggaaaagtctgtatgtggaacaacctataaagcactaaaggaaattgttgagcgtgttttccagtcaaattactttgacagcactcacaaccaccagaatgggctatgtgaggaagaagaggcagcctcagcacctacagttgaagaccaggtagctgaagctgagcctgagccagcagaagaatacactgaacaaagtgaagttgaatcaacagagtatgtaaatagacaatttatggcagaaacacagttcagcagtggtgaaaaggagcaggtagatgagtggacggtcgaaacagtggaggtggtgaattcactccagcagcaacctcaggctgcgtctccttcagtaccagagccccactctttgactccggtggctcaggcagatccccttgtgagaagacagcgagtccaggaccttatggcgcagatgcaggggccctataatttcatacaggattcaatgctggattttgaaaaccagacactcgatcctgccattgtatctgcacagcctatgaatccgacacaaaacatggacatgccccagctggtttgccctccagttcattctgaatctagacttgctcaacctaatcaagttcctgtacaaccagaagctacacaggttcctttggtttcatccacaagtgaggggtatacagcatctcaacccttgtaccagccttctcatgctacagagcaacgaccacaaaaggaaccaattgaccagattcaggcaacaatctctttaaatacagaccagactacagcgtcatcatcccttccggctgcttctcagcctcaggtattccaggctgggacaagcaaaccattacatagcagtggaatcaatgtaaatgcagctccattccaatccatgcaaacggtgttcaatatgaatgccccagttcctcctgttaatgaaccagaaactttgaaacaacaaaatcagtaccaggccagttataaccagagcttttctagtcagcctcaccaagtagaacaaacagaccttcagcaagaacagcttcaaacagtggttggcacttaccatggttcccaggaccagccccaccaagtgactggtaaccatcagcagcctccccagcagaacactggatttccacgtagcagtcagccctattacaatagtcgtggtgtgtctcgtggtggttcccgtggtgctagaggcttaatgaatggatacaggggccctgccaatggattcagaggaggatatgatggttaccgcccttcattctctaacactccaaacagtggttatacacagtctcagttcagtgctccccgggactactctggctatcagcgggatggatatcagcagaatttcaagcgaggctctgggcagagtggaccacggggagccccacgaggtcgtggagggcccccaagacccaacagagggatgccgcaaatgaacactcagcaagtgaattaatctgattcacaggattatgtttaaacgccaaaaacacactggccagtgtaccataatatgttaccagaagagttattatctatttggactgttttcatcccataaagacaggactacaattgtcagc

SEQ ID NO: 14 - protein, 717 aa, Canis lupus; identical to SEQ ID NO: 8
  source 1..717: note = Canis familiaris

SEQ ID NO: 15 - DNA, 3386 bp, Bos taurus
  source 1..3386: mol_type = other DNA
  CDS 82..2208: protein_id = 317; translation = MPSATSHSGSGSKSSGPPPPSGSSGNEAGAGAAAPASQHPMTGT GAVQTEAMKQILGVIDKKLRNLEKKKGKLDDYQERMNKGERLNQDQLDAVSKYQEVTN NLEFAKELQRSFMALSQDIQKTIKKTARREQLMREEAEQKRLKTVLELQYVLDKLGDD EVRTDLKQGLNGVPILSEEELSLLDEFYKLADPERDMSLRLNEQYEHASIHLWDLLEG KEKPVCGTTYKALKEIVERVFQSNYFDSTHNHQNGLCEEEEAASAPTVEDQAAEAEPE PVEEYTEQNEVESTEYVNRQFMAETQFSSGEKEQVDDWTVETVEVVNSLQQQPQAASP SVPEPHSLTPVAQADPLVRRQRVQDLMAQMQGPYNFIQDSMLDFENQTLDPAIVSAQP MNPAQNMDIPQLVCPPVHSESRLAQPNQVSVQPEATQVPLVSSTSEGYTASQPLYQPS HATDQRPQKEPIDQIQATISLNTDQTTASSSLPAASQPQVFQAGTSKPLHSSGINVNA APFQSMQTVFNMNAPVPPVNEPETLKQQNQYQASYNQSFSSQPHQVEQTELQQEQLQT VVGTYHGSQDQPHQVTGNHQQPPQQNTGFPRSNQPYYNSRGVSRGGSRGARGLMNGYR GPANGFRGGYDGYRPSFSTNTPNSGYTQSQFSAPRDYSGYQRDGYQQNFKRGSGQSGP RGAPRGRGGPPRPNRGMPQMNTQQVN
cgcgtctcgccccgtccaccgattgactcgccgctcttgtccttcctcccgctctttcttctctccccttacggtttcaagatgccttcggccaccagccacagcggaagcggcagcaagtcgtccggaccgccaccgccgtcgggttcctccgggaatgaggcgggggccggggccgccgcgccggcttcccaacaccccatgaccggcaccggggctgtccagaccgaggccatgaagcagattctcggggtgatcgacaagaaacttcggaacctggagaagaaaaagggcaagcttgatgattatcaggaacgaatgaacaaaggggaaaggcttaatcaagatcagctggatgccgtgtctaagtaccaggaagtcacaaataacttggagtttgcaaaagaattacagaggagtttcatggcattaagccaagatattcagaaaacaataaagaagacagcacgtcgggagcagcttatgagagaggaagctgaacagaaacgtttaaaaacagtacttgagctgcagtatgttttggacaaactaggagatgatgaagtgagaactgacctgaagcaaggtttgaatggagtgccaatattgtctgaagaggagttgtcgttgttagatgagttctacaaattagcagaccctgaacgagacatgagcttgaggttgaatgagcagtatgaacatgcctccattcacctgtgggacttgctggaaggaaaggaaaaacctgtatgtggaacaacttataaagctctaaaggaaattgttgagcgtgttttccagtcaaactactttgacagcacccacaaccaccagaatggtctgtgtgaggaagaggaggcagcctcagcacctacagttgaagaccaggcagctgaagctgaacctgagccagtggaagaatatactgaacaaaatgaggttgaatcaacagagtatgtaaatagacaatttatggcagaaacacagttcagcagtggtgaaaaggagcaggtagatgattggacagttgaaacagttgaggtggtaaattcactccagcagcaacctcaggctgcatctccttcagtaccagaaccccactctttgaccccagtggctcaagccgatcccctcgtgagaagacagcgagtacaggaccttatggcacaaatgcaggggccctataatttcatacaggattcaatgttggattttgaaaaccagacacttgatcctgccattgtatctgcacagccgatgaatccagcacagaacatggacataccccagctggtttgccctccagttcattctgaatctagacttgctcaacctaatcaagtttctgtacagccagaagctacacaggttcctttggtttcatccacaagtgagggatatacagcatctcaacccttgtaccaaccttctcatgctactgaccaacgaccacaaaaggaaccgattgatcagattcaggcgacgatctctttaaatacagaccagactacagcatcatcatcccttcctgctgcttctcagcctcaagtgttccaggctgggacaagcaaacctttacatagcagtggaatcaatgtaaatgcagctccattccaatccatgcaaacggtattcaatatgaatgccccagttcctcctgttaatgaaccagaaactttaaaacagcaaaatcagtaccaggccagttacaaccagagcttttccagtcagcctcaccaagtagaacaaacagagcttcagcaagaacagcttcaaacagtggttggcacttatcatggttctcaggaccagccccatcaagtgactggtaaccaccagcagcctcctcagcagaacactggatttccacgtagcaatcagccctattacaacagtcgtggtgtgtctcgtggaggttcccgtggtgctagaggcttgatgaatggatacagaggacctgctaatggattcagaggaggatatgatggttaccgcccttcattctctactaacactccaaacagtggttatacacaatctcaattcagtgctccccgggactactctggctatcagcgggatggatatcagcagaatttcaagcgaggctctgggcagagtggaccacggggagccccacgaggtcgtggagggcccccaagacccaacagagggatgccgcaaatgaacactcagcaagtgaattaatctgattcacaggattatgtttaatcgccaaaaacacactggccagtgtaccataatatgttaccagaagagttattatctatttgttctccctttcaggaaacttattgtaaagggactgttttcatcccataaagacaggactacaattgtcagctttatattacctggatatggaaggaaactatttttactctgcatgttctgtcctaagcgtcatcttgagccttgcacatgatactcagattcctcacccttgcttaggagtaaaacataatatactttaatggggtgatatctccatagttatttgaagtggcttggataaagcaagactgacttctgacattggataaaatctacaaatcagccctagagtcattcagtggtaactgacaaaactaaaatatttcccttgaaaggaagatggaaggagtggagtgtggtttggcagaacaactgcatttcacagcttttccacttaaattggagcactgaacatttagatgcataccgaattatgcatgggccctaatcacacagacaaggctggtgccagccttaggcttgacacggcagtgttcaccctctggccagacgactgtggttcaagacacatgtaaattgctttttaacagctgatactgtataagacaaagccaaaatgcaaaattaggctttgattggcacttttcgaaaaatatgcaacaattaagggatataatctggatggccgcttctgtacttaatgtgaaatatttagatacctttcaaacacttaacagtttctttgacaatgagttttgtaaggattggtagtaaatatcattccttatgacgtacattgtctgtcactaatccttggatcttgctgtattgtcacctaaattggtacaggtactgatgaaaatctaatggataatcataacactcttggttacatgtttttcctgcagcctgaaagtttttataagaaaaagacatcaaatgcctgctgctgccacccttttaaattgctatcttttgaaaagcaccagtatgtgttttagattgatttccctattttagggaaatgacagtcagtagtttcacttctgatggtataagcaaacaaataaaacatgtttataaaaaaaaaaaaaaaaaaaaaaaaaaaaaaaaaaaaaaaaaaaaaaaaa

SEQ ID NO: 16 - protein, 708 aa, Bos taurus
MPSATSHSGSGSKSSGPPPPSGSSGNEAGAGAAAPASQHPMTGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDEVRTDLKQGLNGVPILSEEELSLLDEFYKLADPERDMSLRLNEQYEHASIHLWDLLEGKEKPVCGTTYKALKEIVERVFQSNYFDSTHNHQNGLCEEEEAASAPTVEDQAAEAEPEPVEEYTEQNEVESTEYVNRQFMAETQFSSGEKEQVDDWTVETVEVVNSLQQQPQAASPSVPEPHSLTPVAQADPLVRRQRVQDLMAQMQGPYNFIQDSMLDFENQTLDPAIVSAQPMNPAQNMDIPQLVCPPVHSESRLAQPNQVSVQPEATQVPLVSSTSEGYTASQPLYQPSHATDQRPQKEPIDQIQATISLNTDQTTASSSLPAASQPQVFQAGTSKPLHSSGINVNAAPFQSMQTVFNMNAPVPPVNEPETLKQQNQYQASYNQSFSSQPHQVEQTELQQEQLQTVVGTYHGSQDQPHQVTGNHQQPPQQNTGFPRSNQPYYNSRGVSRGGSRGARGLMNGYRGPANGFRGGYDGYRPSFSTNTPNSGYTQSQFSAPRDYSGYQRDGYQQNFKRGSGQSGPRGAPRGRGGPPRPNRGMPQMNTQQVN

SEQ ID NO: 17 - DNA, 3150 bp, Equus caballus
  source 1..3150: mol_type = other DNA
  CDS 1..1917: protein_id = 318; translation = MEGKLDDYQERMNKGERLNQDQLDAVSKYQEVTNNLEFAKELQR SFMALSQDIQKTIKKTARREQLMREEAEQKRLKTVLELQYVLDKLGDEEVRTDLKQGL NGVPILSEEELSLLDEFYKLADPVRDMSLRLNEQYEHASIHLWDLLEGKEKSVCGTTY KALREIVERVFQSNYFDSTHNHQNGLCEEEEATSAPTAEDQGAEAEPEPAEEYTEQSE VESTEYVNRQFMAEAQFSGEKEQVDEWTVETVEVVNSLQQQPQAASPSVPEPHSLTPV AQADPLVRRQRVQDLMAQMQGPYNFIQDSMLDFENQTLDPAIVSAQPMNPAQNMDMPQ LVCPPVHAESRLAQPNQVPVQPEATQVPLVSSTSEGYTASQPLYQPSHATEQRPQKEP TDQIQATISLNTDQTTASSSLPAASQPQVFQAGTSKPLHSSGINVNAAPFQSMQTVFN MNAPVPPVNEPETLKQQNQYQASYNQSFSSPPHQVEQTELPQEQLQTVVGTYHASQDQ PHQVTGNHQQPPQQNTGFPRSSQPYYNSRGVSRGGSRGARGLMNGYRGPANGFRGGYD GYRPSFSNTPNSGYTQSQFSAPRDYSGYQRDGYQQNFKRGSGQSGPRGAPRGRGGPPR PNRGMPQMNTQQVN
atggagggcaagctcgatgattaccaagagcgaatgaacaaaggagaaaggcttaatcaggatcagctggatgctgtgtctaagtaccaggaagtcacaaataacttggagtttgcgaaagaattgcagaggagtttcatggcgttgagtcaggatattcagaaaacaataaagaagacggcacgtcgggagcagcttatgagagaagaagctgaacagaaacgtttaaaaactgtacttgagctgcagtatgttttggacaaattgggagatgaagaagtgcgaactgacctgaaacaaggtttgaatggagtgccaatactctctgaagaagagttgtcgctgttggatgagttctacaagttagcagaccctgtacgggacatgagcttgaggttgaatgagcagtatgagcatgcctccattcacctgtgggacttgctggaagggaaggaaaaatctgtctgtggaacaacctataaagctctgagggaaattgttgagcgtgttttccagtccaactactttgacagcacccacaaccaccagaatgggctctgtgaggaggaagaggctacctcagctccaacagctgaagaccagggagctgaagctgaacctgagccagcagaagaatacactgaacaaagtgaagttgaatcaacagagtatgtaaatagacagtttatggcagaagcgcagttcagtggtgagaaggagcaggtggatgagtggacagtcgagacggtcgaggtggtaaattcactccagcagcaacctcaggctgcatctccttcagtaccggagccccactctttgactccagtggctcaggcagatccccttgtgagaagacagcgagtacaggaccttatggcgcaaatgcaggggccctataatttcatacaggattcaatgctggattttgaaaaccagacacttgatcctgccattgtatctgcacagcctatgaatccagcacagaatatggacatgccccagctggtttgccctccagttcatgctgaatctagacttgctcaacctaatcaagttcctgtacaaccagaagctacacaggttcctttggtttcatccacaagtgaggggtatacagcatctcagcccttgtaccagccttctcatgctacagagcaacgaccgcaaaaggaaccgactgaccagatccaggcaacaatctctttaaatacagaccagactacagcatcatcatcccttcctgctgcttctcagcctcaggtgttccaggctgggacaagcaaacctttacacagcagtgggatcaatgtaaatgcagcgccattccagtccatgcaaacggtgttcaacatgaatgccccggttcctcctgttaatgaaccagaaactttaaaacagcaaaatcagtaccaggccagctataaccagagcttttccagtccgcctcaccaagtagagcagacagagcttccgcaagagcagcttcagacggtggttggtacttaccatgcttcccaagaccagccccatcaagtgaccggtaaccaccagcagcctccccagcagaacactgggtttccacgtagcagtcagccctattacaacagtcgtggtgtgtctcgtggaggctcccgtggtgctagaggcttgatgaatggatacaggggccctgccaatggattcagaggaggatatgatggttaccgcccttcgttctctaacactccaaacagcggttacacacagtctcagttcagtgctccccgggactactctggctatcagcgggatggatatcagcagaatttcaagcgaggctctgggcagagtggaccccggggagccccacgaggtcgtggagggcccccaagacccaacagagggatgccgcaaatgaacactcagcaagtgaattaatctgattcacaggattatctttaatcgccaaaacacactggccagtgtaccataatatgttaccagaagagttattatctatttgttctccctttcaggaaacttattgtaaagggactgttttcatcccataaagacaggactacagttgtcagctttatattacctggatatggaaggaaactatttttactctgcatgttctgtcctaagcgtcatcttgagccttgcacatgatactcagattcctttcccttgcttaggagtaaaacataatatactttatggggtgataatatctccatagttatttgaagtggcttggaaaaagcaagattgacttttgacattggataaaatctacaaatcagccctagagtttcatggtcattcacaaaactaaaatatttcccttgaaaggaagatggaaggactggagtgtggtttggcagaacaactgcatttcacagcttttcctattaaattggagcactgaatgttaaatgcataccaaattatgcatgggcccttaatcacacatacatggctaccagctttgacacagcactattcatcctctggccaaacgactgtggttaaaaacacgtgtaaattgctttttaacagctgatactgtaaaagacaaagctaaaatgcaaaattaggctttcattggcacttttcgaaaaatatgcaacaaatttgggatgtaatctggatggccacttctgtacttaatgtgaagtatttagatacctttttgaacacttaacagtttcttcgacaatgacttttgtaaggattggtagtatatatcattccttatgacatacattgtctgttgctaatccttggatcttgctgtattgtcacctaaattggtacaggtactgatgaaaatctctcatggataaacctaacactcttcgtcacatgtttttcctgcagcctgaaggtttttaaaaggaaaagatatcaaatgcctgctgctaccacccttttaaattgctatcttttgaaaagcaccagtatgtgtttttagattgatttccctattttagggaaatgacagtcagtagtttcagttctgatggtataagcaaagcaaataaaacgtgtttataaaagttgtatcttgaaacactggtgttcaacagctagcagcttctgtggttcaccccctgccttgttagtgttacccatttatggttatctccagcagcaatttctcta

SEQ ID NO: 18 - protein, 638 aa, Equus caballus
MEGKLDDYQERMNKGERLNQDQLDAVSKYQEVTNNLEFAKELQRSFMALSQDIQKTIKKTARREQLMREEAEQKRLKTVLELQYVLDKLGDEEVRTDLKQGLNGVPILSEEELSLLDEFYKLADPVRDMSLRLNEQYEHASIHLWDLLEGKEKSVCGTTYKALREIVERVFQSNYFDSTHNHQNGLCEEEEATSAPTAEDQGAEAEPEPAEEYTEQSEVESTEYVNRQFMAEAQFSGEKEQVDEWTVETVEVVNSLQQQPQAASPSVPEPHSLTPVAQADPLVRRQRVQDLMAQMQGPYNFIQDSMLDFENQTLDPAIVSAQPMNPAQNMDMPQLVCPPVHAESRLAQPNQVPVQPEATQVPLVSSTSEGYTASQPLYQPSHATEQRPQKEPTDQIQATISLNTDQTTASSSLPAASQPQVFQAGTSKPLHSSGINVNAAPFQSMQTVFNMNAPVPPVNEPETLKQQNQYQASYNQSFSSPPHQVEQTELPQEQLQTVVGTYHASQDQPHQVTGNHQQPPQQNTGFPRSSQPYYNSRGVSRGGSRGARGLMNGYRGPANGFRGGYDGYRPSFSNTPNSGYTQSQFSAPRDYSGYQRDGYQQNFKRGSGQSGPRGAPRGRGGPPRPNRGMPQMNTQQVN

SEQ ID NO: 19 - DNA, 6181 bp, Mus musculus
  source 1..6181: mol_type = other DNA
  CDS 179..2302: protein_id = 319; translation = MPSATSHSGSGSKSSGPPPPSGSSGSEAAAGAAAPASQHPATGT GAVQTEAMKQILGVIDKKLRNLEKKKGKLDDYQERMNKGERLNQDQLDAVSKYQEVTN NLEFAKELQRSFMALSQDIQKTIKKTARREQLMREEAEQKRLKTVLELQYVLDKLGDD DVRTDLKQGLSGVPILSEEELSLLDEFYKLVDPERDMSLRLNEQYEHASIHLWDLLEG KEKPVCGTTYKALKEIVERVFQSNYFDSTHNHQNGLCEEEEAASAPTVEDQVAEAEPE PAEEYTEQSEVESTEYVNRQFMAETQFSSGEKEQVDEWTVETVEVVNSLQQQPQAASP SVPEPHSLTPVAQSDPLVRRQRVQDLMAQMQGPYNFIQDSMLDFENQTLDPAIVSAQP MNPTQNMDMPQLVCPQVHSESRLAQSNQVPVQPEATQVPLVSSTSEGYTASQPLYQPS HATEQRPQKEPMDQIQATISLNTDQTTASSSLPAASQPQVFQAGTSKPLHSSGINVNA APFQSMQTVFNMNAPVPPANEPETLKQQSQYQATYNQSFSSQPHQVEQTELQQDQLQT VVGTYHGSQDQPHQVPGNHQQPPQQNTGFPRSSQPYYNSRGVSRGGSRGARGLMNGYR GPANGFRGGYDGYRPSFSNTPNSGYSQSQFTAPRDYSGYQRDGYQQNFKRGSGQSGPR GAPRGRGGPPRPNRGMPQMNTQQVN
gctggctggctaagtccctcccgcgccggctcttgtcccactaggagcagctcagagccgcggggacagggcgaagcggcctgcgcccacggagcgcacgtctctgttctcaacgcagcaccacccttgcccccctcggctgcccactccagacgtccagcggctccgcgcgcgcacgatgccctcggccaccagccacagcggaagcggcagcaaatcgtcgggaccgccgccgccgtccggttcctccgggagtgaggcggcggccggggcagctgcgccggcttctcagcatccggcaaccggcaccggcgccgtccagaccgaggccatgaagcagattctcggcgtaatcgacaagaaacttcggaacctggagaagaaaaagggtaaacttgatgattaccaggaacgaatgaataaaggggaaaggctcaatcaagaccagctggatgccgtatctaagtaccaggaagtcacaaataatttggagtttgcaaaggaattacagaggagtttcatggcattaagtcaagatattcagaaaacaataaagaagacagcacgtcgggaacagcttatgagagaagaagcagaacagaagcgcttaaaaactgtacttgagttacagtatgtattggataagctgggagatgatgatgtgagaacagatctgaaacaaggtttgagtggagtgccaatattgtctgaggaggagttgtcattgctggatgagttctacaagctcgtagatcctgagcgtgacatgagtttaaggttaaatgagcagtatgaacatgcctcaattcacttgtgggatttgctggaagggaaagaaaagcctgtgtgtggaacaacctataaagctctaaaggaaattgttgagcgtgttttccagtcaaactactttgatagcactcacaatcatcaaaatgggttgtgtgaggaggaagaggcggcttcagcgcccacagtggaggaccaggtagctgaagctgaacctgagccagcggaagaatacacagagcaaagtgaggttgaatcaacagagtatgtcaataggcagttcatggcagaaacacagttcagcagtggtgagaaggagcaagtggatgagtggacagttgaaacagttgaggttgtaaactcactccagcagcaacctcaggctgcgtccccttcagtcccagagccccactctttgactccagtggctcagtcagatccacttgtgagaaggcagcgtgtacaagatcttatggcacaaatgcaagggccctataatttcatacaggattcaatgttggattttgaaaatcagacgcttgatcctgccattgtatccgcacagcctatgaaccctacccagaacatggatatgcctcagctggtttgccctcaggttcattctgaatctagacttgcccaatctaatcaagttcctgtacaaccagaagccacacaggttcctttggtttcatccacaagtgaggggtatacagcatctcagcccttgtaccagccatctcatgctacggagcagcggccgcagaaagagccaatggatcagattcaggcaacaatatctttgaatacagaccagactacagcatcctcatcccttcctgctgcttctcagcctcaagtgttccaggctgggacaagtaaacctttgcacagcagtggaatcaatgtaaatgcagctccattccagtccatgcaaacggtgttcaatatgaatgctccagtccctcctgctaatgaaccagaaacgttaaaacaacagagtcagtaccaggccacttataaccagagtttttccagtcagcctcaccaagtggaacaaacagagcttcaacaagaccaactgcaaacggtggttggcacttaccatggatcccaggaccagcctcatcaagtgcctggtaaccaccagcaacccccacagcagaacactggctttccacgtagcagtcagccttattacaacagtcgtggggtatctcgaggagggtctcgtggtgccagaggcttgatgaatggatacaggggccctgccaatggatttagaggaggatatgatggttaccgcccttcattctcgaacactccaaacagtggttattcacagtctcagttcactgctccccgggactactctggttaccagcgggatggatatcagcagaatttcaagcgaggctctgggcagagtggaccacggggagccccacgaggtcgtggagggcccccaagacccaacagagggatgccgcaaatgaacactcagcaagtgaattaatgtgatacacaggattatgtttaatcgccaaaaacacactggccagtgtaccataatatgttaccagaagagttattatctatttgttctccctttcaggaaacttattgtaaagggactgttttcatcccataaagacaggactgcaattgtcagctttacattacctggatatggaaggaaactatttttattctgcatgttctgtcctaagcgtcatcttgagccttgcacacaatacaatactcagattcctcacccttgcttaggagtaaaacattatatacttatggggtgataatatctccatagttagttgaagtggcttggaaaaaaaatgcaagattgaatttttgaccttggataaaatctacaatcagccctagaactattcagtggtaattgacaaagttaaagcattttctttgaaaggaagatggaaggagtggagtgtggtttagcaaaactgcatttcatagctttcccattaaattggagcaccgacagattaaaagcataccaaattatgcatgggtccttactcacacaagtgaggctggctaccagccttgacatagcactcactagtcttctggccaaacgactgtgattaaaacacatgtaaattgctctttagtagtggatactgtgtaagacaaagccaaattgcaaatcaggctttgattggctcttctggaaaatatgcatcaaatatgggggataatctggatgggctgctgctgtgctcaatgtgaactatttagatacctttggaacacttaacagtttctctgaacaatgacttacatggggattggtcctgtttgtcattcctcaccataattgcattgtcatcactaatccttggatcttgctgtattgttactcaaattggtaataggtactgatggaaatcgctaatggatggataatcataacacttttggtcacatgttttctcctgcagcctgaaagttcttaaagaaaaagatatcaaatgcctgctgctaccacccttttaaattgctatctttagaaaagcaccggtatgtgttttagattcatttccctgttttagggaaatgacaggcagtagtttcagttctgatggcaaaacaaataaaaacatgtttctaaaagttgtatcttgaaacactggtgttcaacagctagcagctaaagtaattcaacccatgcattgctagtgtcacagcctttggttatgtctagtagctgtttctgaagtattttcatttatcttttgtcaaatttaaccctgtttgaattctctcctttcctcaaggagacacttatgttcaaagtgttgattctttgccttaggtgcatagagagtagacagtttggagatggaaaggttagcagtgacttagccatatgttctgtgttggaatttgtgctagcagtttgagcactagctctgcgtgcctatgaactgaatgctgcttgtcccattccattttatgtcatggagaaataattccacttggtaacacaaaggctaagttaatgttattttctgtacagaaattaaattttacttttagccttttgtaaactttttttttttttttccaagccggtatcagctactcaaaacaattctcagatattcatcattagacaactggagtttttgctggttttgtagcctactaaaactgctgaggctgttgaacattccacattcaaaagttttgtagggtggtggataatggggaagcttcaatgtttattttaaaataaataaaataagttcttgacttttctcatgtgtggttatggtacatcatattggaagggttatctgtttacttttgccaagactattttgccagcacctacacttgtgtgctttaaaagacaactacctgggatgtaccacaaccatatgttaattgtattttattgggatggataaaatgtttgtggtttattggataatccctagatggtgtgttacgtgtgtagaatataattttatgatagtaagaaagcaaaattgaagaaaataagtttagtattgaatttgagttctgaagtgaattcagggaatgtctcacgtttcgggcttctacccaaagtgtagggcagaaggtgtaaaagttgtttgtagtttgacttgtttattttttaagttgcttattcctttcaacagcaacatatcattagctgtcattctaccattgcagttctagtgagttttaacgtctgcattcaagactgttttaaaagcaacctcactggacagagaactgctaaagtcttttccttaagatctgagtctttgttactcagtatcttctataatatgcaaatgcttgtctagaggcagaagaccttttgtttggtcaagtgtgtattttaccagagtacagggaactgatggtcctacatgtctcttagtgtagtaagactataaaatcttttgtacatgcacaattcacagtatgtttagataccacgtgtataatgcccccccctcccccaggtagcatgccattgatgactttttgcttagggccattttattaccagggccttaatattcctaaaaagatgattttttttcatcctttctcctcttttgatcattgtatcttgatattaaaaacatgaccttccaatgattgtagtaaattaacttctatagttcttttgtctctatatgtattcatatatatgctattgtatagagacttcaaggagacatggagatgcatgcttattctcaggttcattcactaaggtgcttggcagacaaccagtttctaagtgcagaatgtagttaagcagcttcatatatgtgccaggcaatttgttttgttaaattttcatctacttaaggaaatagggtattgtagcttaggctgatcatacccttcatttcaaccttaagctctcaacctgcatccatccgacttgagctattaagtactttagttttatcgagtataagttaacagaaaaagtaaattaagctttgcctttactattttgaatttatatacattctggaaaaacttagaaactgttgtatatttcattagattaaattatatgaaaatgtgattgtttatagcaaagcctgtgagttgcatacaccctaaggaaaactccttaagtgctccttgaagagagaagaaacaattctgggtctggtctttttaagaacaaagctagactactgtatgttagcactgtacattaatagtctgttgtgaagcttgagcagtttcctgcatagccttgatccttcaccgttggcattgaaaatagcagtatccctgatgtacttaaaacttaaagtcaggttttggtatatttatttgtaagtcttaatttcctctaaatactatatctctttagcgagacaacctgaaatttattagcacatttgggtatctcttgcttggcattatggccagtgttaactattcagtggtgaaaaaattacccctcaagacactggagtgaccccagatgtgtgtagtaagtggcatggttcaactgtgtggttaatgataaatatatgacttagtcggtatgatctggaaagacttgattgaaagataattcagctgacataaggatgagtgaggagtggcaaactggataaaagagtcaagagacctgtattccagtgactcctgttttgtttaagcattagcaagatctgtctggggaaactggatagggcagttttcttccatgtttagtttttgtctcaacatttggaagctattgaaggttttaaaatggtgtgtattgtttttttttggggggggggtggccagaatagtgggtcatctaataaaactgccatttaaaagatcaaaaaaaaaaaaaaaaaaaaaaaaa

SEQ ID NO: 20 - protein, 707 aa, Mus musculus
MPSATSHSGSGSKSSGPPPPSGSSGSEAAAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDDVRTDLKQGLSGVPILSEEELSLLDEFYKLVDPERDMSLRLNEQYEHASIHLWDLLEGKEKPVCGTTYKALKEIVERVFQSNYFDSTHNHQNGLCEEEEAASAPTVEDQVAEAEPEPAEEYTEQSEVESTEYVNRQFMAETQFSSGEKEQVDEWTVETVEVVNSLQQQPQAASPSVPEPHSLTPVAQSDPLVRRQRVQDLMAQMQGPYNFIQDSMLDFENQTLDPAIVSAQPMNPTQNMDMPQLVCPQVHSESRLAQSNQVPVQPEATQVPLVSSTSEGYTASQPLYQPSHATEQRPQKEPMDQIQATISLNTDQTTASSSLPAASQPQVFQAGTSKPLHSSGINVNAAPFQSMQTVFNMNAPVPPANEPETLKQQSQYQATYNQSFSSQPHQVEQTELQQDQLQTVVGTYHGSQDQPHQVPGNHQQPPQQNTGFPRSSQPYYNSRGVSRGGSRGARGLMNGYRGPANGFRGGYDGYRPSFSNTPNSGYSQSQFTAPRDYSGYQRDGYQQNFKRGSGQSGPRGAPRGRGGPPRPNRGMPQMNTQQVN

SEQ ID NO: 21 - DNA, 6141 bp, Mus musculus
  source 1..6141: mol_type = other DNA
  CDS 139..2262: protein_id = 320; translation = MPSATSHSGSGSKSSGPPPPSGSSGSEAAAGAAAPASQHPATGT GAVQTEAMKQILGVIDKKLRNLEKKKGKLDDYQERMNKGERLNQDQLDAVSKYQEVTN NLEFAKELQRSFMALSQDIQKTIKKTARREQLMREEAEQKRLKTVLELQYVLDKLGDD DVRTDLKQGLSGVPILSEEELSLLDEFYKLVDPERDMSLRLNEQYEHASIHLWDLLEG KEKPVCGTTYKALKEIVERVFQSNYFDSTHNHQNGLCEEEEAASAPTVEDQVAEAEPE PAEEYTEQSEVESTEYVNRQFMAETQFSSGEKEQVDEWTVETVEVVNSLQQQPQAASP SVPEPHSLTPVAQSDPLVRRQRVQDLMAQMQGPYNFIQDSMLDFENQTLDPAIVSAQP MNPTQNMDMPQLVCPQVHSESRLAQSNQVPVQPEATQVPLVSSTSEGYTASQPLYQPS HATEQRPQKEPMDQIQATISLNTDQTTASSSLPAASQPQVFQAGTSKPLHSSGINVNA APFQSMQTVFNMNAPVPPANEPETLKQQSQYQATYNQSFSSQPHQVEQTELQQDQLQT VVGTYHGSQDQPHQVPGNHQQPPQQNTGFPRSSQPYYNSRGVSRGGSRGARGLMNGYR GPANGFRGGYDGYRPSFSNTPNSGYSQSQFTAPRDYSGYQRDGYQQNFKRGSGQSGPR GAPRGRGGPPRPNRGMPQMNTQQVN
cccaccgcgcgcgcgcgtagccgcctgcccgcccgcccgctgcgcgttttgtcccgcgtctctccccgtccgtctcctgacttgctggtcttgtccttccctcccgcttttttcctctcctctcttctcggtctaaagatgccctcggccaccagccacagcggaagcggcagcaaatcgtcgggaccgccgccgccgtccggttcctccgggagtgaggcggcggccggggcagctgcgccggcttctcagcatccggcaaccggcaccggcgccgtccagaccgaggccatgaagcagattctcggcgtaatcgacaagaaacttcggaacctggagaagaaaaagggtaaacttgatgattaccaggaacgaatgaataaaggggaaaggctcaatcaagaccagctggatgccgtatctaagtaccaggaagtcacaaataatttggagtttgcaaaggaattacagaggagtttcatggcattaagtcaagatattcagaaaacaataaagaagacagcacgtcgggaacagcttatgagagaagaagcagaacagaagcgcttaaaaactgtacttgagttacagtatgtattggataagctgggagatgatgatgtgagaacagatctgaaacaaggtttgagtggagtgccaatattgtctgaggaggagttgtcattgctggatgagttctacaagctcgtagatcctgagcgtgacatgagtttaaggttaaatgagcagtatgaacatgcctcaattcacttgtgggatttgctggaagggaaagaaaagcctgtgtgtggaacaacctataaagctctaaaggaaattgttgagcgtgttttccagtcaaactactttgatagcactcacaatcatcaaaatgggttgtgtgaggaggaagaggcggcttcagcgcccacagtggaggaccaggtagctgaagctgaacctgagccagcggaagaatacacagagcaaagtgaggttgaatcaacagagtatgtcaataggcagttcatggcagaaacacagttcagcagtggtgagaaggagcaagtggatgagtggacagttgaaacagttgaggttgtaaactcactccagcagcaacctcaggctgcgtccccttcagtcccagagccccactctttgactccagtggctcagtcagatccacttgtgagaaggcagcgtgtacaagatcttatggcacaaatgcaagggccctataatttcatacaggattcaatgttggattttgaaaatcagacgcttgatcctgccattgtatccgcacagcctatgaaccctacccagaacatggatatgcctcagctggtttgccctcaggttcattctgaatctagacttgcccaatctaatcaagttcctgtacaaccagaagccacacaggttcctttggtttcatccacaagtgaggggtatacagcatctcagcccttgtaccagccatctcatgctacggagcagcggccgcagaaagagccaatggatcagattcaggcaacaatatctttgaatacagaccagactacagcatcctcatcccttcctgctgcttctcagcctcaagtgttccaggctgggacaagtaaacctttgcacagcagtggaatcaatgtaaatgcagctccattccagtccatgcaaacggtgttcaatatgaatgctccagtccctcctgctaatgaaccagaaacgttaaaacaacagagtcagtaccaggccacttataaccagagtttttccagtcagcctcaccaagtggaacaaacagagcttcaacaagaccaactgcaaacggtggttggcacttaccatggatcccaggaccagcctcatcaagtgcctggtaaccaccagcaacccccacagcagaacactggctttccacgtagcagtcagccttattacaacagtcgtggggtatctcgaggagggtctcgtggtgccagaggcttgatgaatggatacaggggccctgccaatggatttagaggaggatatgatggttaccgcccttcattctcgaacactccaaacagtggttattcacagtctcagttcactgctccccgggactactctggttaccagcgggatggatatcagcagaatttcaagcgaggctctgggcagagtggaccacggggagccccacgaggtcgtggagggcccccaagacccaacagagggatgccgcaaatgaacactcagcaagtgaattaatgtgatacacaggattatgtttaatcgccaaaaacacactggccagtgtaccataatatgttaccagaagagttattatctatttgttctccctttcaggaaacttattgtaaagggactgttttcatcccataaagacaggactgcaattgtcagctttacattacctggatatggaaggaaactatttttattctgcatgttctgtcctaagcgtcatcttgagccttgcacacaatacaatactcagattcctcacccttgcttaggagtaaaacattatatacttatggggtgataatatctccatagttagttgaagtggcttggaaaaaaaatgcaagattgaatttttgaccttggataaaatctacaatcagccctagaactattcagtggtaattgacaaagttaaagcattttctttgaaaggaagatggaaggagtggagtgtggtttagcaaaactgcatttcatagctttcccattaaattggagcaccgacagattaaaagcataccaaattatgcatgggtccttactcacacaagtgaggctggctaccagccttgacatagcactcactagtcttctggccaaacgactgtgattaaaacacatgtaaattgctctttagtagtggatactgtgtaagacaaagccaaattgcaaatcaggctttgattggctcttctggaaaatatgcatcaaatatgggggataatctggatgggctgctgctgtgctcaatgtgaactatttagatacctttggaacacttaacagtttctctgaacaatgacttacatggggattggtcctgtttgtcattcctcaccataattgcattgtcatcactaatccttggatcttgctgtattgttactcaaattggtaataggtactgatggaaatcgctaatggatggataatcataacacttttggtcacatgttttctcctgcagcctgaaagttcttaaagaaaaagatatcaaatgcctgctgctaccacccttttaaattgctatctttagaaaagcaccggtatgtgttttagattcatttccctgttttagggaaatgacaggcagtagtttcagttctgatggcaaaacaaataaaaacatgtttctaaaagttgtatcttgaaacactggtgttcaacagctagcagctaaagtaattcaacccatgcattgctagtgtcacagcctttggttatgtctagtagctgtttctgaagtattttcatttatcttttgtcaaatttaaccctgtttgaattctctcctttcctcaaggagacacttatgttcaaagtgttgattctttgccttaggtgcatagagagtagacagtttggagatggaaaggttagcagtgacttagccatatgttctgtgttggaatttgtgctagcagtttgagcactagctctgcgtgcctatgaactgaatgctgcttgtcccattccattttatgtcatggagaaataattccacttggtaacacaaaggctaagttaatgttattttctgtacagaaattaaattttacttttagccttttgtaaactttttttttttttttccaagccggtatcagctactcaaaacaattctcagatattcatcattagacaactggagtttttgctggttttgtagcctactaaaactgctgaggctgttgaacattccacattcaaaagttttgtagggtggtggataatggggaagcttcaatgtttattttaaaataaataaaataagttcttgacttttctcatgtgtggttatggtacatcatattggaagggttatctgtttacttttgccaagactattttgccagcacctacacttgtgtgctttaaaagacaactacctgggatgtaccacaaccatatgttaattgtattttattgggatggataaaatgtttgtggtttattggataatccctagatggtgtgttacgtgtgtagaatataattttatgatagtaagaaagcaaaattgaagaaaataagtttagtattgaatttgagttctgaagtgaattcagggaatgtctcacgtttcgggcttctacccaaagtgtagggcagaaggtgtaaaagttgtttgtagtttgacttgtttattttttaagttgcttattcctttcaacagcaacatatcattagctgtcattctaccattgcagttctagtgagttttaacgtctgcattcaagactgttttaaaagcaacctcactggacagagaactgctaaagtcttttccttaagatctgagtctttgttactcagtatcttctataatatgcaaatgcttgtctagaggcagaagaccttttgtttggtcaagtgtgtattttaccagagtacagggaactgatggtcctacatgtctcttagtgtagtaagactataaaatcttttgtacatgcacaattcacagtatgtttagataccacgtgtataatgcccccccctcccccaggtagcatgccattgatgactttttgcttagggccattttattaccagggccttaatattcctaaaaagatgattttttttcatcctttctcctcttttgatcattgtatcttgatattaaaaacatgaccttccaatgattgtagtaaattaacttctatagttcttttgtctctatatgtattcatatatatgctattgtatagagacttcaaggagacatggagatgcatgcttattctcaggttcattcactaaggtgcttggcagacaaccagtttctaagtgcagaatgtagttaagcagcttcatatatgtgccaggcaatttgttttgttaaattttcatctacttaaggaaatagggtattgtagcttaggctgatcatacccttcatttcaaccttaagctctcaacctgcatccatccgacttgagctattaagtactttagttttatcgagtataagttaacagaaaaagtaaattaagctttgcctttactattttgaatttatatacattctggaaaaacttagaaactgttgtatatttcattagattaaattatatgaaaatgtgattgtttatagcaaagcctgtgagttgcatacaccctaaggaaaactccttaagtgctccttgaagagagaagaaacaattctgggtctggtctttttaagaacaaagctagactactgtatgttagcactgtacattaatagtctgttgtgaagcttgagcagtttcctgcatagccttgatccttcaccgttggcattgaaaatagcagtatccctgatgtacttaaaacttaaagtcaggttttggtatatttatttgtaagtcttaatttcctctaaatactatatctctttagcgagacaacctgaaatttattagcacatttgggtatctcttgcttggcattatggccagtgttaactattcagtggtgaaaaaattacccctcaagacactggagtgaccccagatgtgtgtagtaagtggcatggttcaactgtgtggttaatgataaatatatgacttagtcggtatgatctggaaagacttgattgaaagataattcagctgacataaggatgagtgaggagtggcaaactggataaaagagtcaagagacctgtattccagtgactcctgttttgtttaagcattagcaagatctgtctggggaaactggatagggcagttttcttccatgtttagtttttgtctcaacatttggaagctattgaaggttttaaaatggtgtgtattgtttttttttggggggggggtggccagaatagtgggtcatctaataaaactgccatttaaaagatcaaaaaaaaaaaaaaaaaaaaaaaaa

SEQ ID NO: 22 - protein, 707 aa, Mus musculus; identical to SEQ ID NO: 20

SEQ ID NO: 23 - DNA, 6114 bp, Mus musculus
  source 1..6114: mol_type = other DNA
  CDS 139..2235: protein_id = 321; translation = MPSATSHSGSGSKSSGPPPPSGSSGSEAAAGAAAPASQHPATGT GAVQTEAMKQILGVIDKKLRNLEKKKGKLDDYQERMNKGERLNQDQLDAVSKYQEVTN NLEFAKELQRSFMALSQDIQKTIKKTARREQLMREEAEQKRLKTVLELQYVLDKLGDD DVRTDLKQGLSGVPILSEEELSLLDEFYKLVDPERDMSLRLNEQYEHASIHLWDLLEG KEKPVCGTTYKALKEIVERVFQSNYFDSTHNHQNGLCEEEEAASAPTVEDQVAEAEPE PAEEYTEQSEVESTEYVNRQFMAETQFSSGEKEQVDEWTVETVEVVNSLQQQPQAASP SVPEPHSLTPVAQSDPLVRRQRVQDLMAQMQGPYNFIQTLDPAIVSAQPMNPTQNMDM PQLVCPQVHSESRLAQSNQVPVQPEATQVPLVSSTSEGYTASQPLYQPSHATEQRPQK EPMDQIQATISLNTDQTTASSSLPAASQPQVFQAGTSKPLHSSGINVNAAPFQSMQTV FNMNAPVPPANEPETLKQQSQYQATYNQSFSSQPHQVEQTELQQDQLQTVVGTYHGSQ DQPHQVPGNHQQPPQQNTGFPRSSQPYYNSRGVSRGGSRGARGLMNGYRGPANGFRGG YDGYRPSFSNTPNSGYSQSQFTAPRDYSGYQRDGYQQNFKRGSGQSGPRGAPRGRGGP PRPNRGMPQMNTQQVN
cccaccgcgcgcgcgcgtagccgcctgcccgcccgcccgctgcgcgttttgtcccgcgtctctccccgtccgtctcctgacttgctggtcttgtccttccctcccgcttttttcctctcctctcttctcggtctaaagatgccctcggccaccagccacagcggaagcggcagcaaatcgtcgggaccgccgccgccgtccggttcctccgggagtgaggcggcggccggggcagctgcgccggcttctcagcatccggcaaccggcaccggcgccgtccagaccgaggccatgaagcagattctcggcgtaatcgacaagaaacttcggaacctggagaagaaaaagggtaaacttgatgattaccaggaacgaatgaataaaggggaaaggctcaatcaagaccagctggatgccgtatctaagtaccaggaagtcacaaataatttggagtttgcaaaggaattacagaggagtttcatggcattaagtcaagatattcagaaaacaataaagaagacagcacgtcgggaacagcttatgagagaagaagcagaacagaagcgcttaaaaactgtacttgagttacagtatgtattggataagctgggagatgatgatgtgagaacagatctgaaacaaggtttgagtggagtgccaatattgtctgaggaggagttgtcattgctggatgagttctacaagctcgtagatcctgagcgtgacatgagtttaaggttaaatgagcagtatgaacatgcctcaattcacttgtgggatttgctggaagggaaagaaaagcctgtgtgtggaacaacctataaagctctaaaggaaattgttgagcgtgttttccagtcaaactactttgatagcactcacaatcatcaaaatgggttgtgtgaggaggaagaggcggcttcagcgcccacagtggaggaccaggtagctgaagctgaacctgagccagcggaagaatacacagagcaaagtgaggttgaatcaacagagtatgtcaataggcagttcatggcagaaacacagttcagcagtggtgagaaggagcaagtggatgagtggacagttgaaacagttgaggttgtaaactcactccagcagcaacctcaggctgcgtccccttcagtcccagagccccactctttgactccagtggctcagtcagatccacttgtgagaaggcagcgtgtacaagatcttatggcacaaatgcaagggccctataatttcatacagacgcttgatcctgccattgtatccgcacagcctatgaaccctacccagaacatggatatgcctcagctggtttgccctcaggttcattctgaatctagacttgcccaatctaatcaagttcctgtacaaccagaagccacacaggttcctttggtttcatccacaagtgaggggtatacagcatctcagcccttgtaccagccatctcatgctacggagcagcggccgcagaaagagccaatggatcagattcaggcaacaatatctttgaatacagaccagactacagcatcctcatcccttcctgctgcttctcagcctcaagtgttccaggctgggacaagtaaacctttgcacagcagtggaatcaatgtaaatgcagctccattccagtccatgcaaacggtgttcaatatgaatgctccagtccctcctgctaatgaaccagaaacgttaaaacaacagagtcagtaccaggccacttataaccagagtttttccagtcagcctcaccaagtggaacaaacagagcttcaacaagaccaactgcaaacggtggttggcacttaccatggatcccaggaccagcctcatcaagtgcctggtaaccaccagcaacccccacagcagaacactggctttccacgtagcagtcagccttattacaacagtcgtggggtatctcgaggagggtctcgtggtgccagaggcttgatgaatggatacaggggccctgccaatggatttagaggaggatatgatggttaccgcccttcattctcgaacactccaaacagtggttattcacagtctcagttcactgctccccgggactactctggttaccagcgggatggatatcagcagaatttcaagcgaggctctgggcagagtggaccacggggagccccacgaggtcgtggagggcccccaagacccaacagagggatgccgcaaatgaacactcagcaagtgaattaatgtgatacacaggattatgtttaatcgccaaaaacacactggccagtgtaccataatatgttaccagaagagttattatctatttgttctccctttcaggaaacttattgtaaagggactgttttcatcccataaagacaggactgcaattgtcagctttacattacctggatatggaaggaaactatttttattctgcatgttctgtcctaagcgtcatcttgagccttgcacacaatacaatactcagattcctcacccttgcttaggagtaaaacattatatacttatggggtgataatatctccatagttagttgaagtggcttggaaaaaaaatgcaagattgaatttttgaccttggataaaatctacaatcagccctagaactattcagtggtaattgacaaagttaaagcattttctttgaaaggaagatggaaggagtggagtgtggtttagcaaaactgcatttcatagctttcccattaaattggagcaccgacagattaaaagcataccaaattatgcatgggtccttactcacacaagtgaggctggctaccagccttgacatagcactcactagtcttctggccaaacgactgtgattaaaacacatgtaaattgctctttagtagtggatactgtgtaagacaaagccaaattgcaaatcaggctttgattggctcttctggaaaatatgcatcaaatatgggggataatctggatgggctgctgctgtgctcaatgtgaactatttagatacctttggaacacttaacagtttctctgaacaatgacttacatggggattggtcctgtttgtcattcctcaccataattgcattgtcatcactaatccttggatcttgctgtattgttactcaaattggtaataggtactgatggaaatcgctaatggatggataatcataacacttttggtcacatgttttctcctgcagcctgaaagttcttaaagaaaaagatatcaaatgcctgctgctaccacccttttaaattgctatctttagaaaagcaccggtatgtgttttagattcatttccctgttttagggaaatgacaggcagtagtttcagttctgatggcaaaacaaataaaaacatgtttctaaaagttgtatcttgaaacactggtgttcaacagctagcagctaaagtaattcaacccatgcattgctagtgtcacagcctttggttatgtctagtagctgtttctgaagtattttcatttatcttttgtcaaatttaaccctgtttgaattctctcctttcctcaaggagacacttatgttcaaagtgttgattctttgccttaggtgcatagagagtagacagtttggagatggaaaggttagcagtgacttagccatatgttctgtgttggaatttgtgctagcagtttgagcactagctctgcgtgcctatgaactgaatgctgcttgtcccattccattttatgtcatggagaaataattccacttggtaacacaaaggctaagttaatgttattttctgtacagaaattaaattttacttttagccttttgtaaactttttttttttttttccaagccggtatcagctactcaaaacaattctcagatattcatcattagacaactggagtttttgctggttttgtagcctactaaaactgctgaggctgttgaacattccacattcaaaagttttgtagggtggtggataatggggaagcttcaatgtttattttaaaataaataaaataagttcttgacttttctcatgtgtggttatggtacatcatattggaagggttatctgtttacttttgccaagactattttgccagcacctacacttgtgtgctttaaaagacaactacctgggatgtaccacaaccatatgttaattgtattttattgggatggataaaatgtttgtggtttattggataatccctagatggtgtgttacgtgtgtagaatataattttatgatagtaagaaagcaaaattgaagaaaataagtttagtattgaatttgagttctgaagtgaattcagggaatgtctcacgtttcgggcttctacccaaagtgtagggcagaaggtgtaaaagttgtttgtagtttgacttgtttattttttaagttgcttattcctttcaacagcaacatatcattagctgtcattctaccattgcagttctagtgagttttaacgtctgcattcaagactgttttaaaagcaacctcactggacagagaactgctaaagtcttttccttaagatctgagtctttgttactcagtatcttctataatatgcaaatgcttgtctagaggcagaagaccttttgtttggtcaagtgtgtattttaccagagtacagggaactgatggtcctacatgtctcttagtgtagtaagactataaaatcttttgtacatgcacaattcacagtatgtttagataccacgtgtataatgcccccccctcccccaggtagcatgccattgatgactttttgcttagggccattttattaccagggccttaatattcctaaaaagatgattttttttcatcctttctcctcttttgatcattgtatcttgatattaaaaacatgaccttccaatgattgtagtaaattaacttctatagttcttttgtctctatatgtattcatatatatgctattgtatagagacttcaaggagacatggagatgcatgcttattctcaggttcattcactaaggtgcttggcagacaaccagtttctaagtgcagaatgtagttaagcagcttcatatatgtgccaggcaatttgttttgttaaattttcatctacttaaggaaatagggtattgtagcttaggctgatcatacccttcatttcaaccttaagctctcaacctgcatccatccgacttgagctattaagtactttagttttatcgagtataagttaacagaaaaagtaaattaagctttgcctttactattttgaatttatatacattctggaaaaacttagaaactgttgtatatttcattagattaaattatatgaaaatgtgattgtttatagcaaagcctgtgagttgcatacaccctaaggaaaactccttaagtgctccttgaagagagaagaaacaattctgggtctggtctttttaagaacaaagctagactactgtatgttagcactgtacattaatagtctgttgtgaagcttgagcagtttcctgcatagccttgatccttcaccgttggcattgaaaatagcagtatccctgatgtacttaaaacttaaagtcaggttttggtatatttatttgtaagtcttaatttcctctaaatactatatctctttagcgagacaacctgaaatttattagcacatttgggtatctcttgcttggcattatggccagtgttaactattcagtggtgaaaaaattacccctcaagacactggagtgaccccagatgtgtgtagtaagtggcatggttcaactgtgtggttaatgataaatatatgacttagtcggtatgatctggaaagacttgattgaaagataattcagctgacataaggatgagtgaggagtggcaaactggataaaagagtcaagagacctgtattccagtgactcctgttttgtttaagcattagcaagatctgtctggggaaactggatagggcagttttcttccatgtttagtttttgtctcaacatttggaagctattgaaggttttaaaatggtgtgtattgtttttttttggggggggggtggccagaatagtgggtcatctaataaaactgccatttaaaagatcaaaaaaaaaaaaaaaaaaaaaaaaa

SEQ ID NO: 24 - protein, 698 aa, Mus musculus
MPSATSHSGSGSKSSGPPPPSGSSGSEAAAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDDVRTDLKQGLSGVPILSEEELSLLDEFYKLVDPERDMSLRLNEQYEHASIHLWDLLEGKEKPVCGTTYKALKEIVERVFQSNYFDSTHNHQNGLCEEEEAASAPTVEDQVAEAEPEPAEEYTEQSEVESTEYVNRQFMAETQFSSGEKEQVDEWTVETVEVVNSLQQQPQAASPSVPEPHSLTPVAQSDPLVRRQRVQDLMAQMQGPYNFIQTLDPAIVSAQPMNPTQNMDMPQLVCPQVHSESRLAQSNQVPVQPEATQVPLVSSTSEGYTASQPLYQPSHATEQRPQKEPMDQIQATISLNTDQTTASSSLPAASQPQVFQAGTSKPLHSSGINVNAAPFQSMQTVFNMNAPVPPANEPETLKQQSQYQATYNQSFSSQPHQVEQTELQQDQLQTVVGTYHGSQDQPHQVPGNHQQPPQQNTGFPRSSQPYYNSRGVSRGGSRGARGLMNGYRGPANGFRGGYDGYRPSFSNTPNSGYSQSQFTAPRDYSGYQRDGYQQNFKRGSGQSGPRGAPRGRGGPPRPNRGMPQMNTQQVN

SEQ ID NO: 25 - DNA, 3548 bp, Mus musculus
  source 1..3548: mol_type = other DNA
  CDS 179..2257: protein_id = 322; translation = MPSATSHSGSGSKSSGPPPPSGSSGSEAAAGAAAPASQHPATGT GAVQTEAMKQILGVIDKKLRNLEKKKGKLDDYQERMNKGERLNQDQLDAVSKYQEVTN NLEFAKELQRSFMALSQDIQKTIKKTARREQLMREEAEQKRLKTVLELQYVLDKLGDD DVRTDLKQGLSGVPILSEEELSLLDEFYKLVDPERDMSLRLNEQYEHASIHLWDLLEG KEKPVCGTTYKALKEIVERVFQSNYFDSTHNHQNGLCEEEEAASAPTVEDQVAEAEPE PAEEYTEQSEVESTEYVNRQFMAETQFSSGEKEQVDEWTVETVEVVNSLQQQPQAASP SVPEPHSLTPVAQSDPLVRRQRVQDLMAQMQGPYNFIQDSMLDFENQTLDPAIVSAQP MNPTQNMDMPQLVCPQVHSESRLAQSNQVPVQPEATQVPLVSSTSEGYTASQPLYQPS HATEQRPQKEPMDQIQATISLNTDQTTASSSLPAASQPQVFQAGTSKPLHSSGINVNA APFQSMQTVFNMNAPVPPANEPETLKQQSQYQATYNQSFSSQPHQVEQTELQQDQLQT VVGTYHGSQDQPHQVPGNHQQPPQQNTGFPRSSQPYYNSRGVSRGGSRGARGLMNGYR GPANGFRGGYDGYRPSFSNTPNSGYSQSQFTAPRDYSGYQRDGYQQNFKRGSGQSGPR GAPRGNILWW
gctggctggctaagtccctcccgcgccggctcttgtcccactaggagcagctcagagccgcggggacagggcgaagcggcctgcgcccacggagcgcacgtctctgttctcaacgcagcaccacccttgcccccctcggctgcccactccagacgtccagcggctccgcgcgcgcacgatgccctcggccaccagccacagcggaagcggcagcaaatcgtcgggaccgccgccgccgtccggttcctccgggagtgaggcggcggccggggcagctgcgccggcttctcagcatccggcaaccggcaccggcgccgtccagaccgaggccatgaagcagattctcggcgtaatcgacaagaaacttcggaacctggagaagaaaaagggtaaacttgatgattaccaggaacgaatgaataaaggggaaaggctcaatcaagaccagctggatgccgtatctaagtaccaggaagtcacaaataatttggagtttgcaaaggaattacagaggagtttcatggcattaagtcaagatattcagaaaacaataaagaagacagcacgtcgggaacagcttatgagagaagaagcagaacagaagcgcttaaaaactgtacttgagttacagtatgtattggataagctgggagatgatgatgtgagaacagatctgaaacaaggtttgagtggagtgccaatattgtctgaggaggagttgtcattgctggatgagttctacaagctcgtagatcctgagcgtgacatgagtttaaggttaaatgagcagtatgaacatgcctcaattcacttgtgggatttgctggaagggaaagaaaagcctgtgtgtggaacaacctataaagctctaaaggaaattgttgagcgtgttttccagtcaaactactttgatagcactcacaatcatcaaaatgggttgtgtgaggaggaagaggcggcttcagcgcccacagtggaggaccaggtagctgaagctgaacctgagccagcggaagaatacacagagcaaagtgaggttgaatcaacagagtatgtcaataggcagttcatggcagaaacacagttcagcagtggtgagaaggagcaagtggatgagtggacagttgaaacagttgaggttgtaaactcactccagcagcaacctcaggctgcgtccccttcagtcccagagccccactctttgactccagtggctcagtcagatccacttgtgagaaggcagcgtgtacaagatcttatggcacaaatgcaagggccctataatttcatacaggattcaatgttggattttgaaaatcagacgcttgatcctgccattgtatccgcacagcctatgaaccctacccagaacatggatatgcctcagctggtttgccctcaggttcattctgaatctagacttgcccaatctaatcaagttcctgtacaaccagaagccacacaggttcctttggtttcatccacaagtgaggggtatacagcatctcagcccttgtaccagccatctcatgctacggagcagcggccgcagaaagagccaatggatcagattcaggcaacaatatctttgaatacagaccagactacagcatcctcatcccttcctgctgcttctcagcctcaagtgttccaggctgggacaagtaaacctttgcacagcagtggaatcaatgtaaatgcagctccattccagtccatgcaaacggtgttcaatatgaatgctccagtccctcctgctaatgaaccagaaacgttaaaacaacagagtcagtaccaggccacttataaccagagtttttccagtcagcctcaccaagtggaacaaacagagcttcaacaagaccaactgcaaacggtggttggcacttaccatggatcccaggaccagcctcatcaagtgcctggtaaccaccagcaacccccacagcagaacactggctttccacgtagcagtcagccttattacaacagtcgtggggtatctcgaggagggtctcgtggtgccagaggcttgatgaatggatacaggggccctgccaatggatttagaggaggatatgatggttaccgcccttcattctcgaacactccaaacagtggttattcacagtctcagttcactgctccccgggactactctggttaccagcgggatggatatcagcagaatttcaagcgaggctctgggcagagtggaccacggggagccccacgaggtaatatattgtggtggtgatcctagctcctatgtggagcttctgttctggccttggaagaactgttcatagtccgcatgtaggttacatgttaggaatacatttatcttttccagacttgttgctaaagattaaatgaaatgctctgtttctaaaatttcatcttgaatccaaattttaatttttgaatgactttccctgctgttgtcttcaaaatcagaacattttctctgcctcagaaaagcgtttttccaactggaaatttatttttcaggtcttaaaacctgctaaatgtttttaggaagtacctactgaaactttttgtaagacatttttggaacgagcttgaacatttatataaatttattaccctctttgatttttgaaacatgcatattatatttaggctgagaagcccttcaaatggccagataagccacagttttagctagagaaccatttagaattgacataactaatctaaacttgaacacttttaggaccaatgttagtgttctaaataccaacatatttctgatgtttaaacagatctcccaaattcttaggaccttgatgtcattaaaatttagaatgacaagcttaagaggctttagtttcatttgtttttcaagtaatgaaaaataatttcttacatgggcagatagttaatttgttgaacaattacaggtagcatttcatgtaatctgatgttctaaatggttctcttattgaaggaggttaaagaattaggtttcttacagtttttggctggccatgacatgtataaaatgtatattaaggaggaattataaagtactttaatttgaatgctagtggcaattgatcattaagaaagtactttaaagcaaaaggttaatgggtcatctgggaaaaatactgaagtatcaaaggtatttgcatgtgaatgtgggttatgttcttctatcccaccttgtagcatattctatgaaagttgagttaaatgatagctaaaatatctgtttcaacagcatgtaaaaagttattttaactgttacaagtcattatacaattttgaatgttctgtagtttctttttaacagtttaggtacaaaggtctgttttcattctggtgctttttattaattttgatagtatgatgtcacttcctattgaaatgtaagctagcgtgtaccttagaatgtgagctccatgagagcaggtaccttgtttgtcttcactgctgtatctattcccaacgcctcatgacagtgcctggcacatagtaggcactcaataaatacttgttgaatgaatgaaaaaaaaaaaaaa

SEQ ID NO: 26 - protein, 692 aa, Mus musculus
MPSATSHSGSGSKSSGPPPPSGSSGSEAAAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDDVRTDLKQGLSGVPILSEEELSLLDEFYKLVDPERDMSLRLNEQYEHASIHLWDLLEGKEKPVCGTTYKALKEIVERVFQSNYFDSTHNHQNGLCEEEEAASAPTVEDQVAEAEPEPAEEYTEQSEVESTEYVNRQFMAETQFSSGEKEQVDEWTVETVEVVNSLQQQPQAASPSVPEPHSLTPVAQSDPLVRRQRVQDLMAQMQGPYNFIQDSMLDFENQTLDPAIVSAQPMNPTQNMDMPQLVCPQVHSESRLAQSNQVPVQPEATQVPLVSSTSEGYTASQPLYQPSHATEQRPQKEPMDQIQATISLNTDQTTASSSLPAASQPQVFQAGTSKPLHSSGINVNAAPFQSMQTVFNMNAPVPPANEPETLKQQSQYQATYNQSFSSQPHQVEQTELQQDQLQTVVGTYHGSQDQPHQVPGNHQQPPQQNTGFPRSSQPYYNSRGVSRGGSRGARGLMNGYRGPANGFRGGYDGYRPSFSNTPNSGYSQSQFTAPRDYSGYQRDGYQQNFKRGSGQSGPRGAPRGNILWW

SEQ ID NO: 27 - DNA, 3508 bp, Mus musculus
  source 1..3508: mol_type = other DNA
  CDS 139..2217: protein_id = 323; translation = MPSATSHSGSGSKSSGPPPPSGSSGSEAAAGAAAPASQHPATGT GAVQTEAMKQILGVIDKKLRNLEKKKGKLDDYQERMNKGERLNQDQLDAVSKYQEVTN NLEFAKELQRSFMALSQDIQKTIKKTARREQLMREEAEQKRLKTVLELQYVLDKLGDD DVRTDLKQGLSGVPILSEEELSLLDEFYKLVDPERDMSLRLNEQYEHASIHLWDLLEG KEKPVCGTTYKALKEIVERVFQSNYFDSTHNHQNGLCEEEEAASAPTVEDQVAEAEPE PAEEYTEQSEVESTEYVNRQFMAETQFSSGEKEQVDEWTVETVEVVNSLQQQPQAASP SVPEPHSLTPVAQSDPLVRRQRVQDLMAQMQGPYNFIQDSMLDFENQTLDPAIVSAQP MNPTQNMDMPQLVCPQVHSESRLAQSNQVPVQPEATQVPLVSSTSEGYTASQPLYQPS HATEQRPQKEPMDQIQATISLNTDQTTASSSLPAASQPQVFQAGTSKPLHSSGINVNA APFQSMQTVFNMNAPVPPANEPETLKQQSQYQATYNQSFSSQPHQVEQTELQQDQLQT VVGTYHGSQDQPHQVPGNHQQPPQQNTGFPRSSQPYYNSRGVSRGGSRGARGLMNGYR GPANGFRGGYDGYRPSFSNTPNSGYSQSQFTAPRDYSGYQRDGYQQNFKRGSGQSGPR GAPRGNILWW
cccaccgcgcgcgcgcgtagccgcctgcccgcccgcccgctgcgcgttttgtcccgcgtctctccccgtccgtctcctgacttgctggtcttgtccttccctcccgcttttttcctctcctctcttctcggtctaaagatgccctcggccaccagccacagcggaagcggcagcaaatcgtcgggaccgccgccgccgtccggttcctccgggagtgaggcggcggccggggcagctgcgccggcttctcagcatccggcaaccggcaccggcgccgtccagaccgaggccatgaagcagattctcggcgtaatcgacaagaaacttcggaacctggagaagaaaaagggtaaacttgatgattaccaggaacgaatgaataaaggggaaaggctcaatcaagaccagctggatgccgtatctaagtaccaggaagtcacaaataatttggagtttgcaaaggaattacagaggagtttcatggcattaagtcaagatattcagaaaacaataaagaagacagcacgtcgggaacagcttatgagagaagaagcagaacagaagcgcttaaaaactgtacttgagttacagtatgtattggataagctgggagatgatgatgtgagaacagatctgaaacaaggtttgagtggagtgccaatattgtctgaggaggagttgtcattgctggatgagttctacaagctcgtagatcctgagcgtgacatgagtttaaggttaaatgagcagtatgaacatgcctcaattcacttgtgggatttgctggaagggaaagaaaagcctgtgtgtggaacaacctataaagctctaaaggaaattgttgagcgtgttttccagtcaaactactttgatagcactcacaatcatcaaaatgggttgtgtgaggaggaagaggcggcttcagcgcccacagtggaggaccaggtagctgaagctgaacctgagccagcggaagaatacacagagcaaagtgaggttgaatcaacagagtatgtcaataggcagttcatggcagaaacacagttcagcagtggtgagaaggagcaagtggatgagtggacagttgaaacagttgaggttgtaaactcactccagcagcaacctcaggctgcgtccccttcagtcccagagccccactctttgactccagtggctcagtcagatccacttgtgagaaggcagcgtgtacaagatcttatggcacaaatgcaagggccctataatttcatacaggattcaatgttggattttgaaaatcagacgcttgatcctgccattgtatccgcacagcctatgaaccctacccagaacatggatatgcctcagctggtttgccctcaggttcattctgaatctagacttgcccaatctaatcaagttcctgtacaaccagaagccacacaggttcctttggtttcatccacaagtgaggggtatacagcatctcagcccttgtaccagccatctcatgctacggagcagcggccgcagaaagagccaatggatcagattcaggcaacaatatctttgaatacagaccagactacagcatcctcatcccttcctgctgcttctcagcctcaagtgttccaggctgggacaagtaaacctttgcacagcagtggaatcaatgtaaatgcagctccattccagtccatgcaaacggtgttcaatatgaatgctccagtccctcctgctaatgaaccagaaacgttaaaacaacagagtcagtaccaggccacttataaccagagtttttccagtcagcctcaccaagtggaacaaacagagcttcaacaagaccaactgcaaacggtggttggcacttaccatggatcccaggaccagcctcatcaagtgcctggtaaccaccagcaacccccacagcagaacactggctttccacgtagcagtcagccttattacaacagtcgtggggtatctcgaggagggtctcgtggtgccagaggcttgatgaatggatacaggggccctgccaatggatttagaggaggatatgatggttaccgcccttcattctcgaacactccaaacagtggttattcacagtctcagttcactgctccccgggactactctggttaccagcgggatggatatcagcagaatttcaagcgaggctctgggcagagtggaccacggggagccccacgaggtaatatattgtggtggtgatcctagctcctatgtggagcttctgttctggccttggaagaactgttcatagtccgcatgtaggttacatgttaggaatacatttatcttttccagacttgttgctaaagattaaatgaaatgctctgtttctaaaatttcatcttgaatccaaattttaatttttgaatgactttccctgctgttgtcttcaaaatcagaacattttctctgcctcagaaaagcgtttttccaactggaaatttatttttcaggtcttaaaacctgctaaatgtttttaggaagtacctactgaaactttttgtaagacatttttggaacgagcttgaacatttatataaatttattaccctctttgatttttgaaacatgcatattatatttaggctgagaagcccttcaaatggccagataagccacagttttagctagagaaccatttagaattgacataactaatctaaacttgaacacttttaggaccaatgttagtgttctaaataccaacatatttctgatgtttaaacagatctcccaaattcttaggaccttgatgtcattaaaatttagaatgacaagcttaagaggctttagtttcatttgtttttcaagtaatgaaaaataatttcttacatgggcagatagttaatttgttgaacaattacaggtagcatttcatgtaatctgatgttctaaatggttctcttattgaaggaggttaaagaattaggtttcttacagtttttggctggccatgacatgtataaaatgtatattaaggaggaattataaagtactttaatttgaatgctagtggcaattgatcattaagaaagtactttaaagcaaaaggttaatgggtcatctgggaaaaatactgaagtatcaaaggtatttgcatgtgaatgtgggttatgttcttctatcccaccttgtagcatattctatgaaagttgagttaaatgatagctaaaatatctgtttcaacagcatgtaaaaagttattttaactgttacaagtcattatacaattttgaatgttctgtagtttctttttaacagtttaggtacaaaggtctgttttcattctggtgctttttattaattttgatagtatgatgtcacttcctattgaaatgtaagctagcgtgtaccttagaatgtgagctccatgagagcaggtaccttgtttgtcttcactgctgtatctattcccaacgcctcatgacagtgcctggcacatagtaggcactcaataaatacttgttgaatgaatgaaaaaaaaaaaaaa

SEQ ID NO: 28 - protein, 692 aa, Mus musculus; identical to SEQ ID NO: 26

SEQ ID NO: 29 - DNA, 2109 bp, Gallus gallus
  source 1..2109: mol_type = other DNA
  CDS 1..2109: protein_id = 324; translation = MPSATNGTMASSSGKAGPGGNEQAPAAAAAAPQASGGSITSVQT EAMKQILGVIDKKLRNLEKKKSKLDDYQERMNKGERLNQDQLDAVSKYQEVTNNLEFA KELQRSFMALSQDIQKTIKKTARREQLMREEAEQKRLKTVLELQFILDKLGDDEVRSD LKQGSNGVPVLTEEELTMLDEFYKLVYPERDMNMRLNEQYEQASVHLWDLLEGKEKPV CGTTYKALKEVVERILQTSYFDSTHNHQNGLCEEEEAAPTPAVEDTVAEAEPDPAEEF TEPTEVESTEYVNRQFMAETQFSSSEKEQVDEWTVETVEVVNSLQQQTQATSPPVPEP HTLTTVAQADPLVRRQRVQDLMAQMQGPYNFMQDSMLEFENQTLDPAIVSAQPMNPAQ NLDMPQMVCPPVHTESRLAQPNQVPVQPEATQVPLVSSTSEGYTASQPMYQPSHTTEQ RPQKESIDQIQASMSLNADQTPSSSSLPTASQPQVFQAGSSKPLHSSGINVNAAPFQS MQTVFNMNAPVPPVNEPEALKQQNQYQASYNQSFSNQPHQVEQSDLQQEQLQTVVGTY HGSPDQTHQVAGNHQQPPQQNTGFPRNSQPYYNSRGVSRGGSRGTRGLMNGYRGPANG FRGGYDGYRPSFSNTPNSGYTQPQFNAPRDYSNYQRDGYQQNFKRGSGQSGPRGAPRG RGGPPRPNRGMPQMNAQQVN
atgccctcggctaccaacggcaccatggcgagcagcagcgggaaggcgggcccgggcggcaacgagcaggccccggcggcggcagcggcggccccgcaggcgtcgggcggcagcatcacctcggttcagaccgaggccatgaagcagatcttgggagtgatcgacaaaaagctccgcaacctcgagaagaaaaagagcaaacttgacgattaccaggaacgaatgaacaagggggaacgtctaaatcaagatcaactggatgcagtgtcaaaataccaggaagtgacaaataacctggaattcgctaaagaactgcagaggagctttatggcactgagccaagatatccagaaaacaataaaaaagacggctcgcagggagcagctgatgagagaagaggctgagcagaagcgtttaaagactgtgctagagctgcagttcattttggacaagttgggtgacgatgaagtgcgcagtgacttgaaacaaggatcaaatggagtaccggtactgacagaggaggaactgacaatgctggatgaattttacaagctagtttaccctgaaagggacatgaacatgaggttgaatgagcagtatgagcaagcatctgttcacctgtgggacttactggaagggaaggaaaaacccgtttgtggaacaacctataaagccctgaaggaggttgttgaacgtattcttcaaactagttactttgatagcacccataaccatcagaacgggttatgtgaggaagaagaggcagcacccacacctgcagtagaagacactgtagcagaagctgagcctgatccagcagaagaatttactgaacctactgaagttgaatcgactgagtatgtaaacagacaattcatggcagagactcagttcagcagtagtgagaaggaacaggtagatgagtggacagttgaaacggttgaggttgtaaattcactgcagcaacaaacacaagctacatctcctccagttcctgaacctcatacactcactactgtggctcaagcagatcctcttgttagaagacagagagtacaggaccttatggcccagatgcagggtccatataacttcatgcaggactctatgctggagtttgagaaccagacacttgatcctgccattgtatctgcacagcccatgaatccagcacagaatttggacatgccgcaaatggtctgccctccagttcatactgagtcaagacttgcccagcctaatcaagttcctgtgcaaccagaagctacgcaggttcccttggtttcatctacaagtgagggatatacagcctcccagcccatgtatcagccttctcataccacagagcaacggccacagaaggaatccattgaccagattcaggcttcaatgtcactgaatgcagaccagaccccgtcatcatcatcacttcccactgcatcccagccgcaagttttccaagctggatctagcaaacctttgcatagcagcggaatcaatgttaatgcagctccattccaatccatgcaaacagtattcaacatgaatgcacctgttcctcctgttaatgagccagaagcccttaagcaacaaaatcagtaccaggccagttacaaccagagtttctccaatcagccacaccaagtagaacaatcagatcttcagcaagaacagctccagacagtggttggtacttaccatggttctccggaccagacccatcaagtggcaggaaaccaccagcaacctccccagcagaatactggatttccacgcaacagtcagccttattacaacagtcggggagtgtctcgtggtggatcacgtgggactcgtggattgatgaatggttacaggggacctgcaaatggatttagaggaggatatgatggctaccgtccttcattttccaacactccgaacagtggttacacgcagccccaatttaatgctcctcgagattattcaaactaccagcgggatggatatcagcagaacttcaaacgtggttctggacaaagtgggcctcggggagctcctcgaggtcgtggagggcccccaagaccaaacagagggatgcctcaaatgaacgctcagcaagtgaattaa

SEQ ID NO: 30 - protein, 702 aa, Gallus gallus
MPSATNGTMASSSGKAGPGGNEQAPAAAAAAPQASGGSITSVQTEAMKQILGVIDKKLRNLEKKKSKLDDYQERMNKGERLNQDQLDAVSKYQEVTNNLEFAKELQRSFMALSQDIQKTIKKTARREQLMREEAEQKRLKTVLELQFILDKLGDDEVRSDLKQGSNGVPVLTEEELTMLDEFYKLVYPERDMNMRLNEQYEQASVHLWDLLEGKEKPVCGTTYKALKEVVERILQTSYFDSTHNHQNGLCEEEEAAPTPAVEDTVAEAEPDPAEEFTEPTEVESTEYVNRQFMAETQFSSSEKEQVDEWTVETVEVVNSLQQQTQATSPPVPEPHTLTTVAQADPLVRRQRVQDLMAQMQGPYNFMQDSMLEFENQTLDPAIVSAQPMNPAQNLDMPQMVCPPVHTESRLAQPNQVPVQPEATQVPLVSSTSEGYTASQPMYQPSHTTEQRPQKESIDQIQASMSLNADQTPSSSSLPTASQPQVFQAGSSKPLHSSGINVNAAPFQSMQTVFNMNAPVPPVNEPEALKQQNQYQASYNQSFSNQPHQVEQSDLQQEQLQTVVGTYHGSPDQTHQVAGNHQQPPQQNTGFPRNSQPYYNSRGVSRGGSRGTRGLMNGYRGPANGFRGGYDGYRPSFSNTPNSGYTQPQFNAPRDYSNYQRDGYQQNFKRGSGQSGPRGAPRGRGGPPRPNRGMPQMNAQQVN

SEQ ID NO: 31 - protein, 63 aa, Homo sapiens
EEYTEQSEVESTEYVNRQFMAETQFTSGEKEQVDEWTVETVEVVNSLQQQPQAASPSVPEPHS

SEQ ID NO: 32 - protein, 18 aa, Homo sapiens
VFNMNAPVPPVNEPETLK

SEQ ID NO: 33 - protein, 16 aa, Homo sapiens
ATQVPLVSSTSEGYTA

SEQ ID NO: 34 - protein, 25 aa, Homo sapiens
QILGVIDKKLRNLEKKKGKLDDYQE

SEQ ID NO: 35 - protein, 23 aa, Homo sapiens
PRGRGGPPRPNRGMPQMNTQQVN

SEQ ID NO: 36 - protein, 5 aa, Gallus gallus
SYQMN

SEQ ID NO: 37 - protein, 17 aa, Gallus gallus
AINKFGNSTGHGAAVKG

SEQ ID NO: 38 - protein, 19 aa, Gallus gallus
HAYGYCGSGTWCAAGEIDA

SEQ ID NO: 39 - protein, 128 aa, Gallus gallus
AVTLDESGGGLQMSRGGLSLVCKASGFDFSSYQMNWIRQAPGKGLEFVAAINKFGNSTGHGAAVKGRVTISRDNGQSTVRLQLNNLRAEDTAIYFCTKHAYGYCGSGTWCAAGEIDAWGHGTEVIVSS

SEQ ID NO: 40 - protein, 9 aa, Gallus gallus
SGGGSYSYG

SEQ ID NO: 41 - protein, 7 aa, Gallus gallus
NNKRPSD

SEQ ID NO: 42 - protein, 10 aa, Gallus gallus
SGDSTDTAVF

SEQ ID NO: 43 - protein, 108 aa, Gallus gallus
QAASTQPSSVSANPGETVEITCSGGGSYSYGWFQQKSPGSAPVTVIYYNNKRPSDIPSRFSGSKSGSTGTLTITGVQADDEAVYYCGSGDSTDTAVFGAGTTLTVLGQ

SEQ ID NO: 44 - protein, 5 aa, Oryctolagus cuniculus
SHSLG

SEQ ID NO: 45 - protein, 16 aa, Oryctolagus cuniculus
DIRSGGSAYYANWAKG

SEQ ID NO: 46 - protein, 13 aa, Oryctolagus cuniculus
TNGPSDLTNRLDL

SEQ ID NO: 47 - protein, 121 aa, Homo sapiens
EQSLVESGGGLVQPGGSLRLSCAASGFSLSSHSLGWVRQAPGKGLEWIGDIRSGGSAYYANWAKGRFTISRDNSKNTLYLQMNSLRAEDTAVYYCTRTNGPSDLTNRLDLWGQGTLVTVSS

SEQ ID NO: 48 - protein, 13 aa, Oryctolagus cuniculus
QASQSLYNNENLA

SEQ ID NO: 49 - protein, 7 aa, Oryctolagus cuniculus
GASTLAS

SEQ ID NO: 50 - protein, 13 aa, Oryctolagus cuniculus
LGEFSCGSADCFA

SEQ ID NO: 51 - protein, 112 aa, Homo sapiens
QVLTQSPSSLSASVGDRVTINCQASQSLYNNENLAWFQQKPGKVPKRLIYGASTLASGVSSRFSGSGSGTEFTLTISSLQCEDFAIYYCLGEFSCGSADCFAFGGGTKVEIK

SEQ ID NO: 52 - protein, 4 aa, Gallus gallus
FDMG

SEQ ID NO: 53 - protein, 17 aa, Gallus gallus
QINDAGSRTWYATAVKG

SEQ ID NO: 54 - protein, 12 aa, Gallus gallus
GSGYVGAGAIDA

SEQ ID NO: 55 - protein, 120 aa, Gallus gallus
AVTLDESGGGLQTPGGGLSLVCKASGFTFSSFDMGWVRQAPGKGLEFVAQINDAGSRTWYATAVKGRATISRDNGQTTVRLQLNNLRAEDTGTYYCTRGSGYVGAGAIDAWGHGTEVIVS

SEQ ID NO: 56 - protein, 8 aa, Gallus gallus
SGGSGYYG

SEQ ID NO: 57 - protein, 7 aa, Gallus gallus
NDKRPSD

SEQ ID NO: 58 - protein, 10 aa, Gallus gallus
RYDSTDSGIF

SEQ ID NO: 59 - protein, 105 aa, Gallus gallus
AALTQPSSVSANPGETVKITCSGGSGYYGWYQQQKSPGSAPVTVIYQNDKRPSDIPSRFSGSGSGSTNTLTITGVQAEDEAVYFCGRYDSTDSGIFGAGTTLTVL

SEQ ID NO: 60 - protein, 6 aa, Oryctolagus cuniculus
GSYYMS

SEQ ID NO: 61 - protein, 17 aa, Oryctolagus cuniculus
YIYIGDGVTAYANWAKG

SEQ ID NO: 62 - protein, 4 aa, Oryctolagus cuniculus
GNKL

SEQ ID NO: 63 - protein, 112 aa, Oryctolagus cuniculus
QSLEESGGDLVKPGASLTLTCTASGFSFSGSYYMSWVRQAPGKGLEWIAYIYIGDGVTAYANWAKGRFTISKASSTTVTLQMTSLTAADTATYFCARGNKLWGPGTLVTVSS

SEQ ID NO: 64 - protein, 11 aa, Oryctolagus cuniculus
QASQSISSYLA

SEQ ID NO: 65 - protein, 7 aa, Oryctolagus cuniculus
DASNLDS

SEQ ID NO: 66 - protein, 14 aa, Oryctolagus cuniculus
QCTAVSSATIYGNA

SEQ ID NO: 67 - protein, 112 aa, Oryctolagus cuniculus
DVVMTQTPASVEAAVGGTVTIKCQASQSISSYLAWYQQKPGQPPKRLIYDASNLDSGVPSRFKGSGSGTDFTITISDLECADAATYYCQCTAVSSATIYGNAFGGGTEVVVK

SEQ ID NO: 68 - protein, 127 aa, Gallus gallus
AVTLDESGGGLQTPGGALSLVCKASGFTFSGYDMLWVRQAPGKGLEWVAGIGSTGGGTDYGAAVKGRATISRDNGQSTVRLQLNNLRAEDTATYYCAKVAGGCNSGYCRDSPGSIDAWGHGTEVIVS

SEQ ID NO: 69 - protein, 107 aa, Gallus gallus
AVTQQPASVSANPGETVKITCSGGGSRNYYGWYQQKSPGSVPVTVIYYDDQRPSNIPSRFSGALSGSTSTLTITGVQADDEAVYFCGSADSNTYEGSFGAGTTLTVL

SEQ ID NO: 70 - protein, 148 aa, Mus musculus
MEWSGVFIFLLSGTAGVLSEVQLHQFGAELVKPGASVKISCKASGYTFTDYNMDWVKQSHGKSLEWIGDINPNYDSTSYNQKFKGKATLTVDKSSSTAYMELRSLTSEDTAVYYCARSRSYDYEGFAYWGQGTLVTVSAAKTTPPSVY

SEQ ID NO: 71 - protein, 105 aa, Mus musculus
GLFCSVERCHYQLQSSQNLLSIVNRYHYMSGNPPKLLVYPALLIYEASITKSCVPDRFTRSGSGTNFTLTINFVHADDLIFYYCQHNRGSFLPSSSVQVPRRRSN

SEQ ID NO: 72 - protein, 109 aa, Mus musculus
PRASLGVSETLLCTSGFTFTDYYMSWVRQPPGKALEWLGFIRNKANGYTTEYSASVKGRFTISRDNSQSILYLQMNTLRAEDSATYYCARANWAFDYWGQGTTVTVSSK

SEQ ID NO: 73 - protein, 94 aa, Mus musculus
SGDRVSLSCRASQSISNYLHWYQQKSHESPRLLIKYASQSISGIPSRFSGSGSGTDFTLSINSVETEDFGMYFCQQSNSWPYTFGGGTKLEIKQ

SEQ ID NO: 74 - protein, 118 aa, Mus musculus
AAELVRPGTSVKVSCKASGYAFTNYLIVWIKQRPGQGLEWIGVISPGSGGTNYNEKFKGKAILTADKSSSTAYMQLSSLTSDEFAVYFCAREKIYDDYYEGYFDVWGAGPRHLLASLS

SEQ ID NO: 75 - protein, 107 aa, Mus musculus
GTRCDIRLTQTTSSLSASLGDRVTISCSASLGIGNYLNWYQQKPDGTVKLLIYYTSNLHSGVPSRFSGSGSGTDYSLTISNLEPEDIATYYCQHYSKLPLTFGAGPS

SEQ ID NO: 76 - protein, 113 aa, Mus musculus
GAELVRSGASVKMSCKASGYSFTDYNMYWVKQTPGQGLEWIGYIYPGNGGTNYNQKFKGKATLTADTSSSTAYMQISSLTSEDSAVYFCARDYDDGGYAMDYWGQGTTVTVSS

SEQ ID NO: 77 - protein, 117 aa, Mus musculus
LLLWLTGARCDIQMTQSPASLSASVGETVTITCRASGNIHNYLTWYQQKQGKSPQLLVYNAKTLADGVPSRFSGSGSGTQYSLKINRLQPEDFGSYYCQHFWNIPWTFGGGTKLNSR

SEQ ID NO: 78 - protein, 114 aa, Mus musculus
DAELVKPGASVKISCKASGYTFTDHSIHWVQQKPEQGLEWIGYISPGNGNIKYNEKFKGKATLTADKSSSTAYMQLNSLTSEDSAVYFCKRSLGRGGPYYFDYWGQGTTVTVSS

SEQ ID NO: 79 - protein, 108 aa, Mus musculus
DIVLTQAAPSLPVTPGESVSISCRSSKSLLHSNGNTYLYWFLQRPGQSPQLLIYRMSNLASGVPDRFSGSGSGTAFTLRISRVEAEDVGVYYCMQHREYPVTFGSGPN

SEQ ID NO: 80 - protein, 111 aa, Mus musculus
QVQLQQSGAELMKPGASVKISCKATGYTFSSYWIEWVKQRPGHGLEWIGEILPGSGSTNYNEKFKGKATFTADTSSNTAYMQLSSLTSEDSAVYYCASYYWYFDVWAQDHV

SEQ ID NO: 81 - protein, 109 aa, Mus musculus
IVMTQAAFSNPVTLGTSASISCRSSKNLLHSNGITYLYWYLQRPGQSPQLLIYRVSNLASGVPNRFSGSESGTDFTLRISRVEAEDVGVYYCAQLLELPYTSEGTKRWE

SEQ ID NO: 82 - protein, 109 aa, Mus musculus
GGGLVKPGGSLKLSCAASGFAFSSYDMSWIRQTPEKRLEWVAYISSGAGSTYYPDTVKGRFTVSRDNAKNTLYLQMSSLKSEDTAMYYCARHFYRFDYWGQGTTVTVSS

SEQ ID NO: 83 - protein, 113 aa, Mus musculus
LLLCVSGAPGSIVMTQTPKFLLVSAGDRITITCKASQSVSNDVAWYQQKPGQSPKLLIYYASNRYTGVPDRFTGSGYGTDFTFTISTVQAEDLAVYFCQQDDRFPLTFGAGPS

SEQ ID NO: 84 - protein, 112 aa, Mus musculus
QIQLVQSGPELKKPGETVKISCKASGYTFTNYGMNWVKQAPGKGLKWMGWINTYTGEPTYADDFKGRFAFSLETSASTAYLQINNLKNEDTATYFCATGAWFAYWAKDSSRH

SEQ ID NO: 85 - protein, 107 aa, Mus musculus
GVEGDIVMTQSHKFMSTSVGDRVSITCKASQDVGTAVAWYQQKPGQSPKLLIYWASTRHTGVPDRFTGSGSGTDFTLTISNVQSEDLADYFCQQYSSYPLTFGAGPS

SEQ ID NO: 86 - protein, 118 aa, Mus musculus
GGGLVQPGGSMKVSCVASGFSFIDFWMNWVRQSPEKGLEWVAEIRLKSNNYATHYAESVKGRFTISRDDSKSSVYLQMNNLRPEDTGIYYCTSLFYYYDGTSGFAYWGQGTTVTVLLK

SEQ ID NO: 87 - protein, 109 aa, Mus musculus
DIVMTQSPSSLTVTAGEKVTMHCKSSQSLLNSGDQKNYLTWYQQKPGQPPKLLIYWASTRESGVPDRFTGSGSGTDFTLTISSVQAEDLAVYYCQNDYDYPLTFGAGPS

SEQ ID NO: 88 - protein, 148 aa, Mus musculus; identical to SEQ ID NO: 70

SEQ ID NO: 89 - protein, 139 aa, Mus musculus
MSVLTQVLGLLLLWLTGARCDIQMTQSPASLSASVGETVTITCRASGNIHNYLAWYQQKQGKSPQLLVYNAKTLADGVPSRFSGSGSGTQYSLKINSLQPEDFGSYYCQHFWSTLTFGAGTKLELKRADAAPTVSNPYD

SEQ ID NO: 90 - protein, 100 aa, Mus musculus
DILQASGYSFTGYTMNWVKQSHGKNLEWIGLINPYNGGTSYNQKFKGKATLTVDKSSSTAYMELLSLTSEDSAVYYCARWGVWSAMDYWGQGTTVTVSSK

SEQ ID NO: 91 - protein, 90 aa, Mus musculus
DRVSITCKASQNVRTAVAWYQQKPRQSPKALIYLASNRDTGLPDRFPGRGSGTDFTLNITNVQSEDLEDYFCLQHCNYPNEFRGCTKVPI

SEQ ID NO: 92 - protein, 116 aa, Mus musculus
LQESGAELARPGASVKLSCKASGYTFTSYWMQWVKQRPGQGLEWIGAIYPGDGDTRYTQKFKGKATLTADKSSSTAYMQLSSLASEDSAVYYCARGEYGNYFAYWGQGTTVTVSSN

SEQ ID NO: 93 - protein, 100 aa, Mus musculus
TSDASLGERVTITCKASQDINSYLSWFQQKPGKSPKTLIYRANRLVDGVPSRFSGSGSGQDYSLTISSLEYEDMGIYYCLQYDEFPLTFGGGTKLEIKQK

SEQ ID NO: 94 - protein, 108 aa, Mus musculus
AWLSQLSCTASGFNIKDTYMHWVKQRPEQGLEWIGRIDPANGNTKYDPKFQGKATITADTSSNTAYLQLSSLTSEDTAVYYCARPIHYYYGSSLAYWGQGTTVTVSSK

SEQ ID NO: 95 - protein, 104 aa, Mus musculus
EFHAVSLGQRATISCRASESVDSYGNSFMHWYQQKPGQPPKLLIYRASNLESGIPARFSGSGSRTDFTLTINPVEADDVATYYCQQSNEDPGRSEVVPSWRSNK

SEQ ID NO: 96 - protein, 109 aa, Mus musculus; identical to SEQ ID NO: 72

SEQ ID NO: 97 - protein, 94 aa, Mus musculus; identical to SEQ ID NO: 73

SEQ ID NO: 98 - protein, 111 aa, Mus musculus
PACLPGGSLRLSCATSGFTFTDYYMSWVRQPPGKALEWLGFIRNKANGYTTEYSASVKGRFTISRDNSQSILYLQMNTLRAEDSATYYCARAPLLYYAMDYWGQGTTVTVS

SEQ ID NO: 99 - protein, 102 aa, Mus musculus
RLPFYSLEQRATISYRASKNVSTSGYSYMHWNQQKPGQPPKLLIYLVSNLESGVPARFSGSGSGTDFTLNIHPVEEEDAATYYCQHIRELTRSELVPSWKSN

SEQ ID NO: 100 - protein, 101 aa, Mus musculus
VSCKASGYTFTSYWMHWVKQRPGQGLEWIGMIDPSNSETRLNQKFKDKATLNVDKSSNTAYMQLSSLTSEDSAVYYCARGLRHYWYFDVWGQGTTVTVSSK

SEQ ID NO: 101 - protein, 99 aa, Mus musculus
TILWREGPFSYRASKSVSTSGYSYMHWNQQKPGQPPRLLIYLVSNLESGVPARFSGSGSGTDFTLNIHPVEEEDAATYYCQHIRELTRSEEVPSWRSNK

SEQ ID NO: 102 - protein, 110 aa, Mus musculus
GGGLVKPGGSLKLSCAASGFTFSSYGMSWVRQTPEKRLEWVATISSGGSYTYYPDSVKGRFTISRDNAKNTLYLQMSSLRSEDTAMYYCASLASYYFDYWGQGTTLTVSS

SEQ ID NO: 103 - protein, 113 aa, Mus musculus
GARCDVQMIQSPSSLSASLGDIVTMTCQASQGTSINLNWFQQKPGKAPKLLIYGASSLEDGVPSRFSGSCFGTDFTLTISSLEDEDMATYFCLQHSYLPPLTFGAGTKLELKR

SEQ ID NO: 104 - protein, 111 aa, Mus musculus
GPGLVQPSQSLSITCTVSGFSLTTYDLHWVRQSPGKGLEWLGVIWSGGSTDYNAAFISRLSISKDNSKSQVFFKMNSLQANDTAIYYCARNYGYSAWFAYWGQGTLVTVSA

SEQ ID NO: 105 - protein, 118 aa, Mus musculus
PASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELKR

SEQ ID NO: 106 - protein, 114 aa, Mus musculus
GFELKKPGETVKISCKASGYTFTAYSMHWVKQTPGKGLKWLGWINTETGEPTYTDDFKGRFTFSLETSARIAYLQINDLKNEDTATYFCARRIYYFGRGGFDYWGQGTTVTVSS

SEQ ID NO: 107 - protein, 118 aa, Mus musculus
PASSSDVLMTQTPLSLPVRLGDQSSISCRSSQSIVHSNGNTYLEWYLQKPGQSPKLLIYKVSNRFSGVPDRFSGSGSGTDFTLKISRVEPEDLGVYYCFQGSHVPYTSEGDQAEIKLA

SEQ ID NO: 108 - protein, 114 aa, Mus musculus
GGGLVQPGGSMRLSCVASGFTFSNSWFNWVRQSPEKGLEWVAEIRLTSDNYAIYYAESVKGRFTISRDDSKSSVYLQMNNLRAEDTGIYYCTRPETARATFAYWGQGTTVTVSS

SEQ ID NO: 109 - protein, 118 aa, Mus musculus
PASTSDVLMTQTPLSLPVSLGDQASISCRSSQSIVHSNGNTYLEWYLQKPGQSPKVLIYKVFNRFSGVPDRFSGSGSGTDFTLKISRVEAEDLGVYYCFQGSHVPRTFGGGTKLNQTG

SEQ ID NO: 110 - protein, 111 aa, Mus musculus
GPDLVKPGASVKISCKASGYSFTAYYMHWVKQSHGKSLEWIGRVNPNNGGTTYNQKFKGKAILTVDKSSSTAYMELRSLTFEDSAVYYCARRIYYGYFDYWGQGTTVTVSS

SEQ ID NO: 111 - protein, 104 aa, Mus musculus
AFFAVSLGQRATISCKASQSVDYDGDSYMNWYQQKPGQPPKLLIYVASNLESGVPARFSGSGSGTDFTLNIHPVEEEDAATYYCQQSNEDPYTFGGGTKLEIKQ

SEQ ID NO: 112 - protein, 106 aa, Mus musculus
GAELVKPGASVKLSCTASGLNIRDIYMHWVKQRPEQGLEWIGKIDPANGNTKYDPKFQGKATITADTSSNTAYVQLSSLTSEDTAVYYCAGTGDYWGQGTTVTVSS

SEQ ID NO: 113 - protein, 112 aa, Mus musculus
GTCGDIVMSQSPSSLAVSAGEKVTMSCKSSQSLLNSRTRKNYLAWVQHKPGQSPRLLIYWASTRESGVPDRFTGSGSGTDFTLTISSVQAEDLAVYYCRQSYNLVTFGAGPS

SEQ ID NO: 114 - protein, 112 aa, Mus musculus
GPELVKPGASVKMSCKASGYTFTSYVMHWVKQKPGQGLEWIGYINPYNDGTKYNEKFKGKATLTSDKSSSTAYMELSSLTSEDSAVYYCARRYYYGSSGGYFDVWAQDHVRT

SEQ ID NO: 115 - protein, 108 aa, Mus musculus
DVQITQSPSYLAASPGETITINCRASKSISKYLAWYQEKPGKTNKLLIYSGSTLQSGIPSRFSGSGSGTDFTLTISSLEPEDFAMYYCQQHNEYPYTFGGGTKLEIKR

SEQ ID NO: 116 - protein, 113 aa, Mus musculus
GPELVKPGASVKISCKASGYSFTGYFMNWVMQSHGKSLEWIGRINPYNGDTFYNQKFKGKATLTVDKSSSTAHMELRSLASEDSAVYYCARRIHYYYGSSYYAMDYWGQEPHH

SEQ ID NO: 117 - protein, 108 aa, Mus musculus
DVQITQSPSYLAASPGETITINCRASKSISKYLAWYQEKPGKTNKLLIYSGSTLQSGIPSRFSGSGSGTDFTLTISSLEPEDFAMYYCQQHNEYPWTFGGGTKLEIKR

SEQ ID NO: 118 - protein, 113 aa, Mus musculus
GAGLVKPGASVKLSCKASGYTFTEYIIHWVKQRSGQGLEWIGWFYPGSGSIKYNEKFKDKATLTADKSSSTVYMELSRLTSEDSAVYFCARHEVYYDYDKSMLWTTGVKNLIR

SEQ ID NO: 119 - protein, 108 aa, Mus musculus
SPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPYTFGGGTKLEIKR

SEQ ID NO: 120 - protein, 113 aa, Mus musculus
GAELVRPGTSVKVSCKASVYAFTNYLIEWVKQRPGQGLEWIGVINPKSGGTKYNEKFRGKATLTADKSSSTAYMQLSSLTSGDSAVYFCAITGTDYWGQGTTLTVSSAKTTPP

SEQ ID NO: 121 - protein, 113 aa, Mus musculus
QGTRCDIQMTQTTSSLSASLGDRVTISCSASQGINNYLNWYQQKPDGTVKLLIYYTSSLRSGVPSRFSGSGSGTDYSLTISNLEPEDVATYYCQQYSKLPRTFGGGTKLEIKR

SEQ ID NO: 122 - protein, 121 aa, Homo sapiens
EVQLVESGGGLVQPGGSLRLSCAASGFSLSSHSLGWVRQAPGKGLEWIGDIRSGGSAYYANWAKGRFTISRDNSKNTLYLQMNSLRAEDTAVYYCTRTNGPSDLTNRLDLWGQGTLVTVSS

SEQ ID NO: 123 - protein, 112 aa, Homo sapiens; identical to SEQ ID NO: 51

SEQ ID NO: 124 - protein, 121 aa, Homo sapiens
EVQLVESGGGLVQPGGSLRLSCAASGFSLSSHSLGWVRQAPGKGLEWIGDIRSGGSAYYANWAKGRFTISRDNSKNTLYLQMNSLRAEDTAVYFCTRTNGPSDLTNRLDLWGQGTLVTVSS

SEQ ID NO: 125 - protein, 112 aa, Homo sapiens; identical to SEQ ID NO: 51

SEQ ID NO: 126 - protein, 121 aa, Homo sapiens; identical to SEQ ID NO: 122

SEQ ID NO: 127 - protein, 113 aa, Homo sapiens
EQVLTQSPSSLSASVGDRVTINCQASQSLYNNENLAWFQQKPGKVPKRLIYGASTLASGVSSRFSGSGSGTEFTLTISNLQPEDFAIYYCLGEFSCGSADCFAFGGGTKVEIK

SEQ ID NO: 128 - protein, 121 aa, Homo sapiens; identical to SEQ ID NO: 124

SEQ ID NO: 129 - protein, 113 aa, Homo sapiens
EIVLTQSPSSLSASVGDRVTINCQASQSLYNNENLAWFQQKPGKVPKRLIYGASTLASGVSSRFSGSGSGTEFTLTISNLQPEDFATYYCLGEFSCGSADCFAFGGGTKVEIK

SEQ ID NO: 130 - protein, 121 aa, Homo sapiens; identical to SEQ ID NO: 122

SEQ ID NO: 131 - protein, 113 aa, Homo sapiens; identical to SEQ ID NO: 129

SEQ ID NO: 132 - protein, 121 aa, Homo sapiens; identical to SEQ ID NO: 124

SEQ ID NO: 133 - protein, 113 aa, Homo sapiens; identical to SEQ ID NO: 127

SEQ ID NO: 134 - protein, 5 aa, Gallus gallus
GYDML

SEQ ID NO: 135 - protein, 17 aa, Gallus gallus
GIGSTGGGTDYGAAVKG

SEQ ID NO: 136 - protein, 19 aa, Gallus gallus
VAGGCNSGYCRDSPGSIDA

SEQ ID NO: 137 - protein, 10 aa, Gallus gallus
SGGGSRNYYG

SEQ ID NO: 138 - protein, 7 aa, Gallus gallus
DDQRPSN

SEQ ID NO: 139 - protein, 11 aa, Gallus gallus
SADSNTYEGSF

SEQ ID NO: 140 - protein, 5 aa, Mus musculus
DYNMD

SEQ ID NO: 141 - protein, 17 aa, Mus musculus
DINPNYDSTSYNQKFKG

SEQ ID NO: 142 - protein, 11 aa, Mus musculus
SRSYDYEGFAY

SEQ ID NO: 143 - protein, 11 aa, Mus musculus
LSIVNRYHYMS

SEQ ID NO: 144 - protein, 6 aa, Mus musculus
EASITK

SEQ ID NO: 145 - protein, 9 aa, Mus musculus
QHNRGSFLP

SEQ ID NO: 146 - protein, 5 aa, Mus musculus
DYYMS

SEQ ID NO: 147 - protein, 17 aa, Mus musculus
RNKANGYTTEYSASVKG

SEQ ID NO: 148 - protein, 9 aa, Mus musculus
ARANWAFDY

SEQ ID NO: 149 - protein, 11 aa, Mus musculus
RASQSISNYLH

SEQ ID NO: 150 - protein, 7 aa, Mus musculus
YASQSIS

SEQ ID NO: 151 - protein, 7 aa, Mus musculus
YASQSIS

SEQ ID NO: 152 - protein, 5 aa, Mus musculus
NYLIV

SEQ ID NO: 153 - protein, 17 aa, Mus musculus
VISPGSGGTNYNEKFKG

SEQ ID NO: 154 - protein, 11 aa, Mus musculus
EKIYDDYYEGY

SEQ ID NO: 155 - protein, 15 aa, Mus musculus
TISCSASLGIGNYLN

SEQ ID NO: 156 - protein, 7 aa, Mus musculus
TSNLHSG

SEQ ID NO: 157 - protein, 9 aa, Mus musculus
HYSKLPLTF

SEQ ID NO: 158 - protein, 5 aa, Mus musculus
DYNMY

SEQ ID NO: 159 - protein, 17 aa, Mus musculus
YIYPGNGGTNYNQKFKG

SEQ ID NO: 160 - protein, 11 aa, Mus musculus
DYDDGGYAMDY

SEQ ID NO: 161 - protein, 15 aa, Mus musculus
SVGETVTITCRASGN

SEQ ID NO: 162 - protein, 7 aa, Mus musculus
NAKTLAD

SEQ ID NO: 163 - protein, 9 aa, Mus musculus
QHFWNIPWT

SEQ ID NO: 164 - protein, 5 aa, Mus musculus
DHSIH

SEQ ID NO: 165 - protein, 17 aa, Mus musculus
YISPGNGNIKYNEKFKG

SEQ ID NO: 166 - protein, 12 aa, Mus musculus
SLGRGGPYYFDY

SEQ ID NO: 167 - protein, 16 aa, Mus musculus
RSSKSLLHSNGNTYLY

SEQ ID NO: 168 - protein, 7 aa, Mus musculus
RMSNLAS

SEQ ID NO: 169 - protein, 9 aa, Mus musculus
MQHREYPVT

SEQ ID NO: 170 - protein, 5 aa, Mus musculus
SYWIE

SEQ ID NO: 171 - protein, 17 aa, Mus musculus
EILPGSGSTNYNEKFKG

SEQ ID NO: 172 - protein, 11 aa, Mus musculus
YYWYFDVWAQD

SEQ ID NO: 173 - protein, 15 aa, Mus musculus
SSKNLLHSNGITYLY

SEQ ID NO: 174 - protein, 7 aa, Mus musculus
RVSNLAS

SEQ ID NO: 175 - protein, 9 aa, Mus musculus
AQLLELPYT

SEQ ID NO: 176 - protein, 5 aa, Mus musculus
SYDMS

SEQ ID NO: 177 - protein, 17 aa, Mus musculus
YISSGAGSTYYPDTVKG

SEQ ID NO: 178 - protein, 11 aa, Mus musculus
HFYRFDYWGQG

SEQ ID NO: 179 - protein, 15 aa, Mus musculus
SAGDRITITCKASQS

SEQ ID NO: 180 - protein, 7 aa, Mus musculus
YASNRYT

SEQ ID NO: 181 - protein, 9 aa, Mus musculus
QQDDRFPLT

SEQ ID NO: 182 - protein, 5 aa, Mus musculus
NYGMN

SEQ ID NO: 183 - protein, 17 aa, Mus musculus
WINTYTGEPTYADDFKG

SEQ ID NO: 184 - protein, 11 aa, Mus musculus
GAWFAYWAKDS

SEQ ID NO: 185 - protein, 15 aa, Mus musculus
SITCKASQDVGTAVA

SEQ ID NO: 186 - protein, 7 aa, Mus musculus
WASTRHT

SEQ ID NO: 187 - protein, 9 aa, Mus musculus
QQYSSYPLT

SEQ ID NO: 188 - protein, 5 aa, Mus musculus
DFWMN

SEQ ID NO: 189 - protein, 19 aa, Mus musculus
EIRLKSNNYATHYAESVKG

SEQ ID NO: 190 - protein, 13 aa, Mus musculus
LFYYYDGTSGFAY

SEQ ID NO: 191 - protein, 17 aa, Mus musculus
KSSQSLLNSGDQKNYLT

SEQ ID NO: 192 - protein, 7 aa, Mus musculus
WASTRES

SEQ ID NO: 193 - protein, 9 aa, Mus musculus
QNDYDYPLT

SEQ ID NO: 194 - protein, 5 aa, Mus musculus
DYNMD

SEQ ID NO: 195 - protein, 17 aa, Mus musculus
DINPNYDSTSYNQKFKG

SEQ ID NO: 196 - protein, 11 aa, Mus musculus
SRSYDYEGFAY

SEQ ID NO: 197 - protein, 11 aa, Mus musculus
RASGNIHNYLA

SEQ ID NO: 198 - protein, 7 aa, Mus musculus
NAKTLAD

SEQ ID NO: 199 - protein, 8 aa, Mus musculus
QHFWSTLT

SEQ ID NO: 200 - protein, 5 aa, Mus musculus
GYTMN

SEQ ID NO: 201 - protein, 16 aa, Mus musculus
NPYNGGTSYNQKFKGK

SEQ ID NO: 202 - protein, 9 aa, Mus musculus
WGVWSAMDY

SEQ ID NO: 203 - protein, 11 aa, Mus musculus
KASQNVRTAVA

SEQ ID NO: 204 - protein, 7 aa, Mus musculus
LASNRDT

SEQ ID NO: 205 - protein, 9 aa, Mus musculus
LQHCNYPNE

SEQ ID NO: 206 - protein, 5 aa, Mus musculus
SYWMQ

SEQ ID NO: 207 - protein, 17 aa, Mus musculus
AIYPGDGDTRYTQKFKG

SEQ ID NO: 208 - protein, 11 aa, Mus musculus
ARGEYGNYFAY

SEQ ID NO: 209 - protein, 11 aa, Mus musculus
KASQDINSYLS

SEQ ID NO: 210 - protein, 7 aa, Mus musculus
RANRLVD

SEQ ID NO: 211 - protein, 9 aa, Mus musculus
LQYDEFPLT

SEQ ID NO: 212 - protein, 5 aa, Mus musculus
DTYMH

SEQ ID NO: 213 - protein, 17 aa, Mus musculus
RIDPANGNTKYDPKFQG

SEQ ID NO: 214 - protein, 14 aa, Mus musculus
ARPIHYYYGSSLAY

SEQ ID NO: 215 - protein, 11 aa, Mus musculus
SVDSYGNSFMH

SEQ ID NO: 216 - protein, 7 aa, Mus musculus
RASNLES

SEQ ID NO: 217 - protein, 9 aa, Mus musculus
QQSNEDPGR

SEQ ID NO: 218 - protein, 5 aa, Mus musculus
DYYMS

SEQ ID NO: 219 - protein, 17 aa, Mus musculus
RNKANGYTTEYSASVKG

SEQ ID NO: 220 - protein, 9 aa, Mus musculus
ARANWAFDY

SEQ ID NO: 221 - protein, 11 aa, Mus musculus
RASQSISNYLH

SEQ ID NO: 222 - protein, 7 aa, Mus musculus
YASQSIS

SEQ ID NO: 223 - protein, 9 aa, Mus musculus
QQSNSWPYT

SEQ ID NO: 224 - protein, 5 aa, Mus musculus
DYYMS

SEQ ID NO: 225 - protein, 17 aa, Mus musculus
RNKANGYTTEYSASVKG

SEQ ID NO: 226 - protein, 12 aa, Mus musculus
ARAPLLYYAMDY

SEQ ID NO: 227 - protein, 11 aa, Mus musculus
NVSTSGYSYMH

SEQ ID NO: 228 - protein, 7 aa, Mus musculus
LVSNLES

SEQ ID NO: 229 - protein, 8 aa, Mus musculus
QHIRELTR

SEQ ID NO: 230 - protein, 5 aa, Mus musculus
SYWMH

SEQ ID NO: 231 - protein, 17 aa, Mus musculus
MIDPSNSETRLNQKFKD

SEQ ID NO: 232 - protein, 12 aa, Mus musculus
ARGLRHYWYFDV

SEQ ID NO: 233 - protein, 11 aa, Mus musculus
SVSTSGYSYMH

SEQ ID NO: 234 - protein, 7 aa, Mus musculus
LVSNLES

SEQ ID NO: 235 - protein, 9 aa, Mus musculus
QHIRELTRS

SEQ ID NO: 236 - protein, 5 aa, Mus musculus
SYGMS

SEQ ID NO: 237 - protein, 14 aa, Mus musculus
WVRQTPEKRLEWVA

SEQ ID NO: 238 - protein, 11 aa, Mus musculus
LASYYFDYWGQ

SEQ ID NO: 239 - protein, 15 aa, Mus musculus
TMTCQASQGTSINLN

SEQ ID NO: 240 - protein, 7 aa, Mus musculus
GASSLED

SEQ ID NO: 241 - protein, 11 aa, Mus musculus
LQHSYLPPLTF

SEQ ID NO: 242 - protein, 5 aa, Mus musculus
TYDLH

SEQ ID NO: 243 - protein, 16 aa, Mus musculus
VIWSGGSTDYNAAFIS

SEQ ID NO: 244 - protein, 11 aa, Mus musculus
NYGYSAWFAYW

SEQ ID NO: 245 - protein, 15 aa, Mus musculus
ASISCRSSQSIVHSN

SEQ ID NO: 246 - protein, 7 aa, Mus musculus
KVSNRFS

SEQ ID NO: 247 - protein, 9 aa, Mus musculus
FQGSHVPLT

SEQ ID NO: 248 - protein, 5 aa, Mus musculus
AYSMH

SEQ ID NO: 249 - protein, 17 aa, Mus musculus
WINTETGEPTYTDDFKG

SEQ ID NO: 250 - protein, 11 aa, Mus musculus
RIYYFGRGGFD

SEQ ID NO: 251 - protein, 15 aa, Mus musculus
SSISCRSSQSIVHSN

SEQ ID NO: 252 - protein, 7 aa, Mus musculus
KVSNRFS

SEQ ID NO: 253 - protein, 9 aa, Mus musculus
FQGSHVPYT

SEQ ID NO: 254 - protein, 5 aa, Mus musculus
NSWFN

SEQ ID NO: 255 - protein, 19 aa, Mus musculus
EIRLTSDNYAIYYAESVKG

SEQ ID NO: 256 - protein, 11 aa, Mus musculus
PETARATFAYW

SEQ ID NO: 257 - protein, 15 aa, Mus musculus
ASISCRSSQSIVHSN

SEQ ID NO: 258 - protein, 7 aa, Mus musculus
KVFNRFS

SEQ ID NO: 259 - protein, 9 aa, Mus musculus
FQGSHVPRT

SEQ ID NO: 260 - protein, 5 aa, Mus musculus
AYYMH

SEQ ID NO: 261 - protein, 17 aa, Mus musculus
RVNPNNGGTTYNQKFKG

SEQ ID NO: 262 - protein, 11 aa, Mus musculus
RIYYGYFDYWG

SEQ ID NO: 263 - protein, 15 aa, Mus musculus
KASQSVDYDGDSYMN

SEQ ID NO: 264 - protein, 7 aa, Mus musculus
VASNLES

SEQ ID NO: 265 - protein, 9 aa, Mus musculus
QQSNEDPYT

SEQ ID NO: 266 - protein, 4 aa, Mus musculus
DIYM

SEQ ID NO: 267 - protein, 17 aa, Mus musculus
KIDPANGNTKYDPKFQG

SEQ ID NO: 268 - protein, 11 aa, Mus musculus
TGDYWGQGTTV

SEQ ID NO: 269 - protein, 15 aa, Mus musculus
TMSCKSSQSLLNSRT

SEQ ID NO: 270 - protein, 7 aa, Mus musculus
WASTRES

SEQ ID NO: 271 - protein, 9 aa, Mus musculus
RQSYNLVTF

SEQ ID NO: 272 - protein, 5 aa, Mus musculus
SYVMH

SEQ ID NO: 273 - protein, 17 aa, Mus musculus
YINPYNDGTKYNEKFKG

SEQ ID NO: 274 - protein, 11 aa, Mus musculus
RYYYGSSGGYF

SEQ ID NO: 275 - protein, 15 aa, Mus musculus
RASKSISKYLAWYQE

SEQ ID NO: 276 - protein, 7 aa, Mus musculus
SGSTLQS

SEQ ID NO: 277 - protein, 9 aa, Mus musculus
QQHNEYPYT

SEQ ID NO: 278 - protein, 5 aa, Mus musculus
GYFMN

SEQ ID NO: 279 - protein, 17 aa, Mus musculus
RINPYNGDTFYNQKFKG

SEQ ID NO: 280 - protein, 11 aa, Mus musculus
RIHYYYGSSYY

SEQ ID NO: 281 - protein, 15 aa, Mus musculus
RASKSISKYLAWYQE

SEQ ID NO: 282 - protein, 7 aa, Mus musculus
SGSTLQS

SEQ ID NO: 283 - protein, 9 aa, Mus musculus
QQHNEYPWT

SEQ ID NO: 284 - protein, 5 aa, Mus musculus
EYIIH

SEQ ID NO: 285 - protein, 17 aa, Mus musculus
WFYPGSGSIKYNEKFKD

SEQ ID NO: 286 - protein, 11 aa, Mus musculus
HEVYYDYDKSM

SEQ ID NO: 287 - protein, 15 aa, Mus musculus
LYSSNQKNYLAWYQQ

SEQ ID NO: 288 - protein, 7 aa, Mus musculus
WASTRES

SEQ ID NO: 289 - protein, 9 aa, Mus musculus
QQYYSYPYT

SEQ ID NO: 290 - protein, 5 aa, Mus musculus
NYLIE

SEQ ID NO: 291 - protein, 19 aa, Mus musculus
VINPKSGGTKYNEKFRGKA

SEQ ID NO: 292 - protein, 11 aa, Mus musculus
TGTDYWGQGTT

SEQ ID NO: 293 - protein, 10 aa, Mus musculus
VTISCSASQG

SEQ ID NO: 294 - protein, 7 aa, Mus musculus
YTSSLRS

SEQ ID NO: 295 - protein, 9 aa, Mus musculus
QQYSKLPRT

SEQ ID NO: 296 - protein, 12 aa, Homo sapiens
FTSGEKEQVDEW

SEQ ID NO: 297 - protein, 16 aa, Homo sapiens
AEQKRLKTVLELQYVL

SEQ ID NO: 298 - protein, 16 aa, Homo sapiens
VERVFQSNYFDSTHNH

SEQ ID NO: 299 - protein, 16 aa, Homo sapiens
FQSMQTVFNMNAPVPP

SEQ ID NO: 300 - protein, 5 aa, Mus musculus
TNAMN

SEQ ID NO: 301 - protein, 17 aa, Mus musculus
RIRSKSNNYATYYADSV

SEQ ID NO: 302 - protein, 11 aa, Mus musculus
DWDGFLYFDYW

SEQ ID NO: 303 - protein, 112 aa, Mus musculus
GGGLVQPKGSLKLSCAASGFTFNTNAMNWVRQAPGKGLEWVARIRSKSNNYATYYADSVKDRFTISRDDSQSMLYLQMNNLKTEDTAMYYCVRDWDGFLYFDYWAKHHLTLF

SEQ ID NO: 304 - protein, 15 aa, Mus musculus
SVSTSGYSYMHWNQQ

SEQ ID NO: 305 - protein, 7 aa, Mus musculus
LVSNLES

SEQ ID NO: 306 - protein, 9 aa, Mus musculus
QHIRELTRS

SEQ ID NO: 307 - protein, 104 aa, Mus musculus
TQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 308 - protein, 14 aa, Homo sapiens
PPVNEPETLKQQNQ

SEQ ID NO: 309 - protein, 12 aa, Homo sapiens
ETLKQQNQYQAS

SEQ ID NO: 310 - protein, 709 aa, Homo sapiens; identical to SEQ ID NO: 2

SEQ ID NO: 311 - protein, 694 aa, Homo sapiens; identical to SEQ ID NO: 4

SEQ ID NO: 312 - protein, 448 aa, Canis lupus; identical to SEQ ID NO: 6
  source 1..448: note = Canis familiaris

SEQ ID NO: 313 - protein, 717 aa, Canis lupus; identical to SEQ ID NO: 8
  source 1..717: note = Canis familiaris

SEQ ID NO: 314 - protein, 702 aa, Canis lupus; identical to SEQ ID NO: 10
  source 1..702: note = Canis familiaris

SEQ ID NO: 315 - protein, 679 aa, Canis lupus; identical to SEQ ID NO: 12
  source 1..679: note = Canis familiaris

SEQ ID NO: 316 - protein, 717 aa, Canis lupus; identical to SEQ ID NO: 8
  source 1..717: note = Canis familiaris

SEQ ID NO: 317 - protein, 708 aa, Bos taurus; identical to SEQ ID NO: 16

SEQ ID NO: 318 - protein, 638 aa, Equus caballus; identical to SEQ ID NO: 18

SEQ ID NO: 319 - protein, 707 aa, Mus musculus; identical to SEQ ID NO: 20

SEQ ID NO: 320 - protein, 707 aa, Mus musculus; identical to SEQ ID NO: 20

SEQ ID NO: 321 - protein, 698 aa, Mus musculus; identical to SEQ ID NO: 24

SEQ ID NO: 322 - protein, 692 aa, Mus musculus; identical to SEQ ID NO: 26

SEQ ID NO: 323 - protein, 692 aa, Mus musculus; identical to SEQ ID NO: 26

SEQ ID NO: 324 - protein, 702 aa, Gallus gallus; identical to SEQ ID NO: 30